(19) United States
(12) Patent Application Publication
(10) Pub. No.: US 2025/0128203 A1
(43) Pub. Date: Apr. 24, 2025

(54) MODIFIED REDOX-ACTIVE SORBENTS FOR ELECTROCHEMICAL CARBON DIOXIDE SEPARATION

(71) Applicant: The Johns Hopkins University, Baltimore, MD (US)

(72) Inventors: Yayuan Liu, Baltimore, MD (US); Xing Li, Baltimore, MD (US)

(21) Appl. No.: 18/924,091

(22) Filed: Oct. 23, 2024

Related U.S. Application Data
(60) Provisional application No. 63/592,345, filed on Oct. 23, 2023.

(51) Int. Cl. B01D 53/32; B01D 53/04; B01J 20/22; C07D 209/34

(52) U.S. Cl. CPC B01D 53/326; B01D 53/04; B01J 20/22; C07D 209/34; B01D 2253/20; B01D 2257/504

(57) ABSTRACT

Isoindigo derivatives and their use as sorbents for the electrochemically mediated carbon capture (EMCC) reversible CO₂ capture and release are disclosed.

### CROSS REFERENCE TO RELATED APPLICATIONS

This application claims priority to U.S. Application No. 63/592,345, filed Oct. 23, 2023, the entire contents of which are incorporated herein by reference.

### BACKGROUND

Efficient carbon dioxide (CO₂) separation technologies are essential for mitigating climate change. Electrochemically mediated carbon capture (EMCC) using redox-tunable sorbents has emerged as a promising alternative to traditional thermochemical methods due to its versatility and energy efficiency. The undesirable linear free-energy relationship between redox potential and CO₂ binding affinity in existing EMCC chemistries, however, makes it fundamentally challenging to optimize key sorbent properties independently via chemical modifications. Thus, new redox-tunable sorbents for CO₂ separation technologies are needed to address these challenges.

### SUMMARY

In some aspects, the presently disclosed subject matter provides a compound of formula (I):

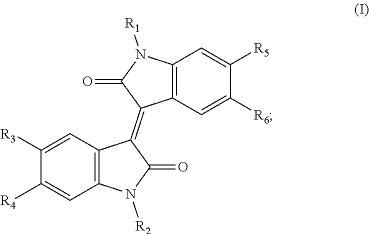

wherein:
R₁ and R₂ are each independently selected from H and branched or straightchain C₁-C₈ alkyl;
R₃, R₄, and R₆ are each independently selected from H, an electron withdrawing group, and an electron donating group;
R₅ is selected from H, an electron withdrawing group, an electron donating group, —COR_{y}, wherein R_{y} is an amino acid or a substituted amino acid, and —C(═O)—OR_{z}, wherein R_{z} is selected from H and branched or straightchain C₁-C₄ alkyl;
provided that at least one of R₁, R₂, R₃, R₄, R₅, and R₆ is not H; and
acceptable salts thereof.

In certain aspects, the electron donating group is selected from alkoxide, hydroxyl, alkoxyl, amine, amide, ester, and alkyl. In certain aspects, the electron withdrawing group is selected from halogen, cyano, nitro, haloalkyl, ammonium, carbonyl, and sulfonyl.

In some aspects, R₁ and R₂ are each independently selected from H and branched or straightchain C₁-C₈ alkyl; R₃, R₄, and R₆ are each independently selected from H, branched or straightchain C₁-C₄ alkyl, halogen, C₁-C₄ alkoxyl, nitro, and —C(═O)—OR₇, wherein R₇ is branched or straightchain C₁-C₄ alkyl; R₅ is selected from H, branched or straightchain C₁-C₄ alkyl, halogen, C₁-C₄ alkoxyl, nitro, —C—OR₈, wherein R₈ is selected from an amino acid, a substituted amino acid, and —NHR₉, wherein R₉ is —CHR₁₀—C(═O)—O—R₁₁, wherein R₁₀ is H or —CH₂—OH, and Ru is branched or straightchain C₁-C₄ alkyl, and —C(═O)—OR₁₂, wherein R₁₂ is selected from H and branched or straightchain C₁-C₄ alkyl; provided that (i) at least one of R₁, R₂, R₃, R₄, R₅, and R₆ is not H, and (ii) R₄ and R₅ cannot both be Br at the same time if R₁, R₂, R₃, and R₆ are each H; and acceptable salts thereof.

In certain aspects, R₁ and R₂ are each H. In other aspects, R₁ and R₂ are each branched or straightchain C₁-C₈ alkyl. In particular aspects, R₁ and R₂ are each —CH₂CH₂—CH₃ or —CH₂CH(CH₂CH₃)(CH₂CH₂CH₂CH₃).

In certain aspects, one or more of R₃, R₄, R₅, and R₆ is selected from branched or straightchain C₁-C₄ alkyl, C₁-C₄ alkoxyl, halogen, nitro, and —C(═O)—OR₁₂, wherein R₁₂ is H or branched or straightchain C₁-C₄ alkyl.

In certain aspects, (i) R₃ and R₆ are each the same and are not H; (ii) R₄ and R₅ are the each same and are not H; or (iii) R₃, R₄, R₅, and R₆ are each the same and are not H.

In certain aspects, (i) R₃ is halogen and R₄, R₅, and R₆ are each H; (ii) R₄ is halogen and R₃, R₅, and R₆ are each H; (iii) R₅ is —C(═O)—OR₁₂, wherein R₁₂ is H or branched or straightchain C₁-C₄ alkyl, R₃ and R₆ are each H, and R₄ is H or halogen; or (iv) R₆ is nitro and R₅ is H, R₃ is H or C₁-C₄ alkoxyl, and R₄ is H or —C(═O)—OR₁₂ wherein R₁₂ is H or branched or straightchain C₁-C₄ alkyl.

In particular aspects, the compound of formula (I) is selected from:

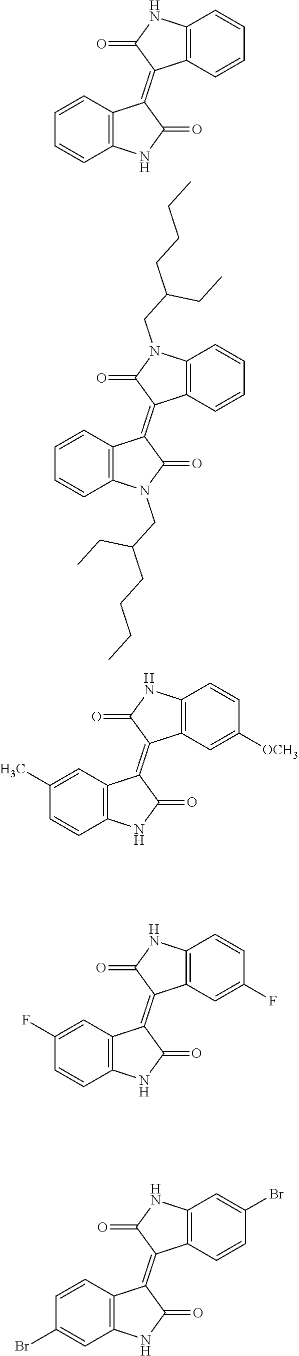 [SMILES: O=C1Nc2ccccc2/C1=C1\C(=O)Nc2ccccc21 ; CCCCC(CC)CN1C(=O)/C(=C2/C(=O)N(CC(CC)CCCC)c3ccccc32)c2ccccc21 ; COc1ccc2c(c1)/C(=C1\C(=O)Nc3ccc(C)cc31)C(=O)N2 ; O=C1Nc2ccc(F)cc2/C1=C1\C(=O)Nc2ccc(F)cc21 ; O=C1Nc2cc(Br)ccc2/C1=C1\C(=O)Nc2cc(Br)ccc21]

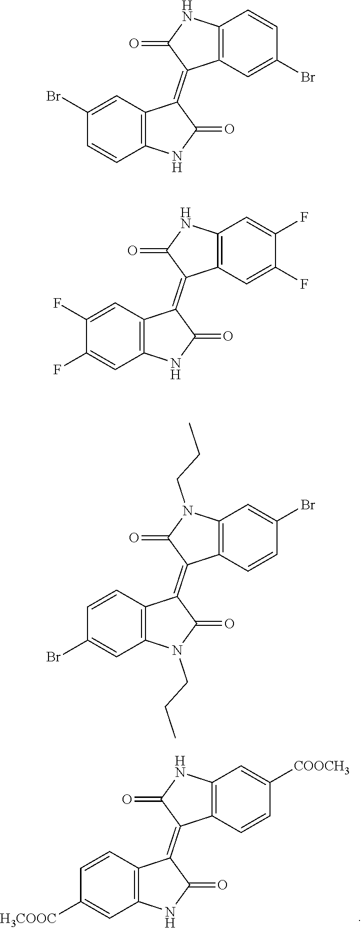

In other aspects, the compound of formula (I) is selected from:

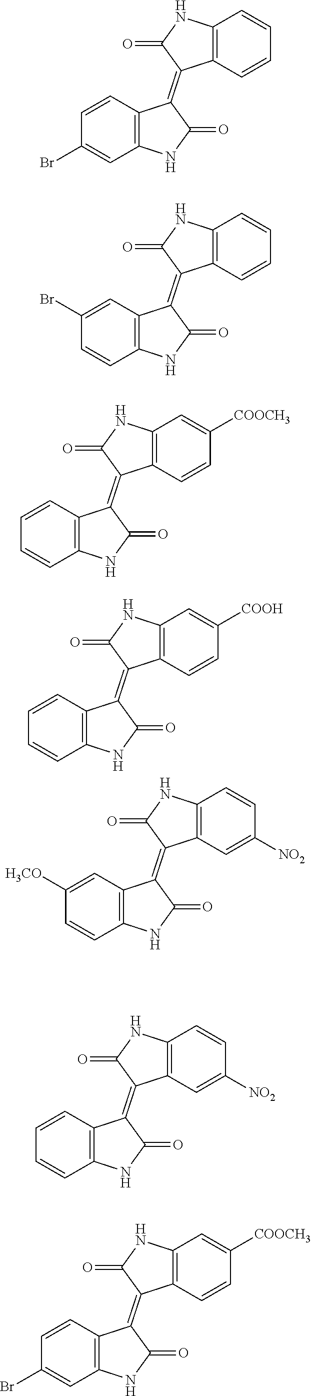

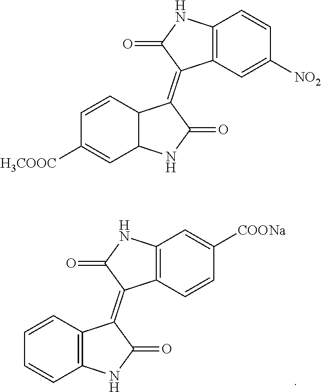

In certain aspects, the compound of formula (I) comprises a compound of formula (Ia):

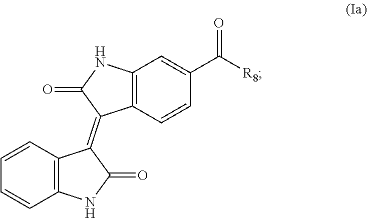

wherein: R₈ is —NHR₉, wherein R₉ is —CHR₁₀—C(═O)—O—R₁₁, wherein R₁₀ is H or —CH₂—OH, and R₁₁ is branched or straightchain C₁-C₄ alkyl. In particular aspects, the compound of formula (Ia) is selected from:

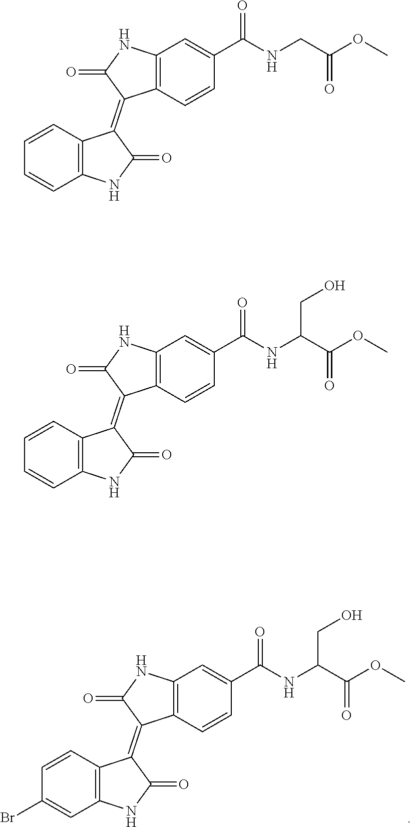 [SMILES: COC(=O)CNC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2cc(Br)ccc21]

In certain aspects, the presently disclosed subject matter provides a sorbent material comprising a compound of formula (I), or in certain aspects, a compound of formula (Ia).

In other aspects, the presently disclosed subject matter provides a process for capturing CO₂ from a gas sample, the process comprising contacting the gas sample with a compound of formula (I), or a sorbent material comprising a compound of formula (I).

In certain aspects, the process comprises an electrochemically mediated carbon capture (EMCC) process. In particular aspects, the process comprises (i) electro-reduction of the sorbent to form an adduct with CO₂; and (ii) oxidising the adduct to liberate CO₂ regenerate the sorbent.

In certain aspects, the gas sample is selected from ambient air, i.e., direct air capture, ventilated air, and a stream of gas, e.g., flue gas or other emissions from a point source, such as an industrial source, such as a chemical plant, petroleum production facility, a cement plant, power generation, and transportation, and from enclosed spaces, for example, in buildings and in the cabin space of cars, trains, airplanes, spacecraft, submarines, and the like.

In certain aspects, the process is a fixed-bed process or a flow-based process.

In other aspects, the presently disclosed subject matter provides a system for separating carbon dioxide from ambient air or a stream of gases, the system comprising the compound of formula (I) or a sorbent material comprising a compound of formula (I).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1a, FIG. 1b, and FIG. 1c show the rational design of bifunctional redox-tunable CO₂ sorbents based on isoindigo and their derivatives. FIG. 1a, a universal designing pattern for redox-tunable CO₂ carriers containing α,β-unsaturated 1,4-diketone functionality. In previous designs, CO₂ is only bonded to the reduced 0 centre via carbonate formation, where the binding affinity is sensitive to structural modification. FIG. 1b, bifunctional redox-tunable CO₂ carrier design based on isoindigo. The secondary functionality of amide allows intramolecular hydrogen bonding, providing an extra handle to stabilise the CO₂ adduct, thereby enhancing the CO₂ binding strength. FIG. 1c, a summary of E_{1/2} of typical quinone-based sorbents and isoindigos under CO₂, and their log K_{CO}_{₂} in DMF (filled and empty dots represent the first and second E_{1/2} under CO₂, respectively);

FIG. 2a and FIG. 2b show validating the redox-tunable CO₂ absorption of isoindigo.

FIG. 2a, bulk electrolysis of isoindigo (IId) under N₂ or CO₂ atmosphere. The CO₂ adduct can be obtained either by directly reducing IId (40 mM) in DMSO with 0.25 M LiClO₄ under CO₂ or by reducing IId under N₂ followed by purging with CO₂. FIG. 2b, ¹H NMR and ¹³C NMR spectra of the crude solution after bulk electrolysis under CO₂. The minor isomer peaks are marked with an asterisk. C, CV curves of IId (2.5 mM) in DMF using 0.1 M Nbu₄PF₆ as the supporting salt under N₂ (grey) and CO₂ (red) at a scan rate of −50 mV s⁻¹ (5 mM ferrocene as the internal reference). ΔEpeak(2) is labelled for the calculation of K_{CO}_{₂};

FIG. 3 shows the modular synthesis of redox-tunable isoindigo sorbents for EMCC. Functional groups can be pre-installed onto the precursors of isoindigos and various isoindigos can be obtained through Knoevenagel condensation of the precursors. Amino acid ester can be further installed through amidation reaction with carboxylic acid modified isoindigos;

FIG. 4a, FIG. 4b, FIG. 4c, FIG. 4d, and FIG. 4e show the structure-property relationships of redox-tunable isoindigo-based CO₂ sorbents. FIG. 4a, CV of various isoindigos using 2.5 mM compound in DMF with 0.1 M NBu₄PF₆ under N₂ (grey) or CO₂ (coloured). The CV curves were recorded at a scan rate of −50 mV s⁻¹ at 298 K. FIG. 4b, DFT-optimised structure of IId; positions for introducing substituent groups are labelled, which are at 5, 6, and lactam N-position; intramolecular hydrogen bonding (a indicates bond length) in the neutral state is shown as blue dashed lines. FIG. 4c, DFT-optimised structure of 5BIId suggests EWG can strengthen the hydrogen bonding (a). FIG. 4d, DFT-optimised structures of 55BIId (left) and 66DBIId (right) showing that EWG substituent at 5-position is more effective in strengthening the hydrogen bonding (a). FIG. 4e, DFT simulation showing that intramolecular hydrogen bonding (b indicates bond length, red dashed lines) occurs in the CO₂ adduct, stabilising the CO₂ binding. Colour of atoms: grey: C; red: O; blue: N; brown: Br; white: H;

FIG. 5a and FIG. 5b show the substituent effect on the redox potentials and CO₂ binding abilities of isoindigos. FIG. 5a, the effect of substituent group on the first electron-transfer half-wave potentials under N₂. FIG. 5b, the plot between the first electron transfer half-wave potential under N₂ and the change in CO₂ binding constant relative to unmodified isoindigo. There is no linear correlation (R₂<0.1) between the relative CO₂ binding constants and the half-wave potentials;

FIG. 6a, FIG. 6b, FIG. 6c, FIG. 6d, and FIG. 6e demonstrates the evaluation of the performance of 6MCIId in the flow-based EMCC prototype. FIG. 6a, Schematic of the flow-based EMCC prototype. FIG. 6b, CO₂ reading at the exit of the sorbent tank over 19 repeating capture/release cycles for approximately 100 hours of operation. FIG. 6c, the CO₂ reading of selected capture/release cycles overlaid, with the cumulative amount of CO₂ captured/released in each cycle relative to the theoretical capacity. Lighter colours represent later cycles. The shaded regions indicate the capture, rest, release, and rest steps. For CO₂ capture, 6MCIId was reduced at 10 mA for 60 min followed by a 75 min rest. For CO₂ release, the adducts were oxidised at 10 mA to 0.05 mV followed by an approximately 120 min constant voltage hold, and finally rested for another 75 min. FIG. 6d, Selected voltage-capacity curves for the 1st, 5th, 10th, and 15th capture/release cycle. FIG. 6e, the CO₂ capacity utilisation efficiency (blue squares), release/capture efficiency (red crosses), and electrochemical efficiency (empty grey squares) of the system. The liquid sorbent was composed of 10 mL 0.1 M 6MCIId in DMF with 0.25 M NaClO₄ as the supporting salt. The sorbent tank was filled with plastic beads and purged with 10% CO₂ at a flow rate of 2 standard cubic centimetres per minute (sccm). On the opposite side, a Fc tank was used to balance the charge, which was filled with 20 mL 0.1 M Fc in DMF with 0.25 M NaClO₄ as the supporting salt, 4 mM ferrocenium tetrafluoroboronate (FcBF₄) to facilitate Fc oxidation, and 10 mM 6MCIId to mitigate sorbent crossover;

FIG. 7 is a diagram showing the current limitations of organic redox-active CO₂ sorbents for EMCC and how the bifunctional isoindigos overcome this constraint;

FIG. 8 shows variable-Temperature ¹HNMR spectra of the crude solution of IId after the bulk reduction in CO₂;

FIG. 9 shows 2D ¹H-¹³C Heteronuclear Single Quantum Coherence (HSQC) NMR of the crude solution after bulk reduction of IId in CO₂;

FIG. 10a and FIG. 10b show the full assignment of the NMR spectra to IId and IId-CO₂. FIG. 10a, ¹HNMR. FIG. 10b, ¹³CNMR;

FIG. 11 shows FT-IR spectra of IId and the IId-CO₂ adduct after bulk electrolysis in DMSO with LiClO₄ as the supporting salt;

FIG. 12 shows a stability test of the isoindigo-CO₂ adduct. Less than 9 mol % of the IId-CO₂ adduct was oxidised back to IId after the solution was stored in the air in the presence of high water content for 53 days (based on ¹H NMR relative integrals);

FIG. 13a, FIG. 13b, FIG. 13c, FIG. 13d, FIG. 13e, FIG. 13f, FIG. 13g, FIG. 13h, FIG. 13i, FIG. 13j, FIG. 13k, FIG. 13l are CVs of isoindigos with various chemical modifications under different atmospheres. FIG. 13a, IId. FIG. 13b, 55DMIId. FIG. 13c, 6BIId. FIG. 13d, 5BIId. FIG. 13e, 66DBIId. FIG. 13f, 55DBIId. FIG. 13g, 6MCIId. FIG. 13h, 6B6MCIId. FIG. 13i, 55DFIId. FIG. 13j, 5566TFIId. FIG. 13k, 5NIId. FIG. 13l, 5M5NIId. The CV was recorded using 2.5 mM compound in anhydrous DMF with 0.1 M NBu₄PF₆ saturated by N₂ (grey), 20% CO₂ (balanced with N₂, blue), and CO₂ (red), respectively, at a scan rate of −50 mV s⁻¹ at 298 K;

FIG. 14a, FIG. 14b, FIG. 14c, FIG. 14d, FIG. 14e, FIG. 14f, FIG. 14g, FIG. 14h, FIG. 14i are CVs of isoindigos with various chemical modifications under different atmospheres. FIG. 14a, 5N6MCIId. FIG. 14b, 66DMCIId. FIG. 14c, NNDEHIId. FIG. 14d, NNDPr66DBIId. FIG. 14e, 6CIId. FIG. 14f, 6CIIdNa. FIG. 14g, 6AIIdGly. FIG. 14h, 6AIIdSer. FIG. 14i, 6B6AIIdSer. The CV was recorded using 2.5 mM compound in anhydrous DMF with 0.1 M NBu₄PF₆ saturated by N₂ (grey), 20% CO₂ (balanced with N₂, blue), and CO₂ (red), respectively, at a scan rate of −50 mV s⁻¹ at 298 K;

FIG. 15 show tabulated half-wave potentials of isoindigos under N₂ or CO₂. Half-wave potentials are summarised from the CVs of various isoindigos using 2.5 mM compound in DMF with 0.1 M NBu₄PF₆ under N₂ (blue) or CO₂ (pink) (filled circle: first redox potential; empty circle: second redox potential). The oxygen reduction reaction to superoxide occurs at −1.35 V vs. Fc⁺/Fc in DMF;

FIG. 16 shows the proposed mechanism for the rotational isomerization of isoindigo radical anion;

FIG. 17 is a CV of 02 saturated DMF with 0.1 M NBu₄PF₆ at a scan rate of −50 mV s⁻¹ with a cathodic sweeping direction initially at 298 K;

FIG. 18 is a characteristic ¹HNMR and half-wave redox potential of 55DBIId and 66DBIId;

FIG. 19, FIG. 20, and FIG. 21 show Kubelka-Munk spectra of various isoindigos;

FIG. 22a and FIG. 22b show the calculation of the reaction rate constant between 6MCIId radical anion and CO₂. FIG. 22a, Plots of current vs. time for 5 mM 6MCIId in DMF with 0.1 M NBu₄PF₆ as the supporting salt for potential steps from open circuit voltage to −0.55 V vs. Ag (V₁) with 20% CO₂, and from open circuit voltage to −1.26 V vs. Ag (V₂) without CO₂. The inset is the CV of the electrolyte under N₂ with the corresponding voltage steps (V₁ and V₂). FIG. 22b, Determination of the forward rate constant (k_{f}) for the addition of CO₂ to 6MCIId^{·-}. The measurements were done in triplicate, and the data is shown for the time interval where the current is between one- and two-electron transfer processes. The slope of the plot gives k_{f};

FIG. 23a and FIG. 23b show the calculation of the reaction rate constant between 6AIIdGly radical anion and CO₂. FIG. 23a, Plots of current vs. time for 5 mM 6AIIdGly in DMF with 0.1 M NBu₄PF₆ as the supporting salt for potential steps from open circuit voltage to −0.6 V vs. Ag (V₁) with 20% CO₂, and from open circuit voltage to −1.282 V vs. Ag (V₂) without CO₂. The inset is the CV of the electrolyte under N₂ with the corresponding voltage steps (V₁ and V₂). FIG. 23b, Determination of the forward rate constant (k_{f}) for the addition of CO₂ to 6AIIdGly^{·-}. The measurements were done in triplicate, and the data is shown for the time interval where the current is between one- and two-electron transfer processes. The slope of the plot gives k_{f};

FIG. 24a and FIG. 24b shows the calculation of the reaction rate constant between 66DBIId radical anion and CO₂. FIG. 24a, Plots of current vs time for 5 mM 66DBIId in DMF with 0.1 M NBu₄PF₆ as the supporting salt for potential steps from open circuit voltage to −0.6 V vs Ag (V₁) with 20% CO₂, and from open circuit voltage to −1.275 V vs Ag (V₂) without CO₂. The inset is the cyclic voltammogram of the electrolyte under N₂ with the corresponding voltage steps (V₁ and V₂). FIG. 24b, Determination of the forward rate constant (k_{f}) for the addition of CO₂ to 66DBIId^{·-}. The measurements were done in triplicate, and the data is shown for the time interval where the current is between that of one- and two-electron transfer processes. The slope of the plot gives k_{f};

FIG. 25a and FIG. 25b show the calculation of the reaction rate constant between NNDPr66DBIId radical anion and CO₂. FIG. 25a, Plots of current vs time for 5 mM NNDPr66DBIId in DMF with 0.1 M NBu₄PF₆ as the supporting salt for potential steps from open circuit voltage to −0.6 V vs Ag (V₁) with 20% CO₂, and from open circuit voltage to −1.252 V vs Ag (V₂) without CO₂. The inset is the cyclic voltammogram of the electrolyte under N₂ with the corresponding voltage steps (V₁ and V₂). FIG. 25b, Determination of the forward rate constant (k_{f}) for the addition of CO₂ to NNDPr66DBIId^{·-}. The measurements were done in triplicate, and the data is shown for the time interval where the current is between that of one- and two-electron transfer processes. The slope of the plot gives k_{f};

FIG. 26a and FIG. 26b are CV curves of 6MCIId under different atmospheres or at different scan rates. FIG. 26a, CV curves under N₂ or 10% CO₂ at a cathodic scan rate of −5 mV s⁻¹. FIG. 26b, CV curves under 10% CO₂ at various scan rates from −5 to −200 mV s⁻¹. The CV curves were recorded using 2.5 mM 6MCIId in anhydrous DMF with 0.1 M NBu₄PF₆;

FIG. 27 shows the proposed mechanism of CO₂ binding inhibition in 5N6MCIId;

FIG. 28 shows the DFT-optimised structures of isoindigos in the neutral state and CO₂-complexed state (colour of atoms: grey: C; red: O; blue: N; brown: Br; white: H);

FIG. 29 shows DFT-optimised structures of isoindigos in the neutral state and CO₂-complexed state (colour of atoms: grey: C; red: O; blue: N; brown: Br; white: H);

FIG. 30 shows CV curves of amino-acid-ester modified isoindigos under different atmospheres. The CV was recorded using 2.5 mM compound in anhydrous DMF with 0.1 M NBu₄PF₆ saturated by N₂ (grey), 10% CO₂ (balanced with N₂, yellow), 20% CO₂ (balanced with N₂, blue), and CO₂ (red), respectively, at a scan rate of −50 mV s⁻¹ at 298 K;

FIG. 31 shows a cytotoxicity test of IId and 6AIIdSer using different mammalian cells. Cells were cultured in the absence or presence of isoindigos under various concentrations from 0.78 to 100 g mL⁻¹ for 48 h. The amino ester modified 6AIIdSer exhibits much better biocompatibility than its unmodified counterparts;

FIG. 32 shows optical microscopic images of mammalian cells after 48 h culture with isoindigos. Different types of cells were cultured in the presence of 50 g mL⁻¹ of IId or 6AIIdSer, and cells remained much healthier under the 6AIIdSer condition. The solvent DMF (1 vol %) was used as the background reference/control. The scale bars are 300 m. NIH3T3/GFP and U2OS.EGFP express green fluorescent protein (GFP);

FIG. 33 shows the impact of 02 on the redox properties and CO₂ binding behaviours of 55DBIId. The CV curves were recorded using 2.5 mM 55DBIId in DMF with 0.1 M NBu₄PF₆ under mixtures of gases at a scan rate of −50 mV s⁻¹ with a cathodic sweeping direction initially at 298 K;

FIG. 34 shows the impact of water on the redox properties and CO₂ binding behaviours of 55DBIId. The CV was recorded using 2.5 mM 55DBIId in DMF with 0.1 M NBu₄PF₆ with various water contents under various atmospheres at a scan rate of −50 mV s⁻¹ with an initial cathodic sweeping direction at 298 K;

FIG. 35 shows the effect of supporting salts on the redox properties and CO₂ binding behaviours of 6MCIId. FIG. 35a, CV curves of 6MCIId in electrolytes with various supporting salt cations under N₂, 20% CO₂, and CO₂, respectively. FIG. 35b, CV curves of 6MCIId in electrolytes with various counter anions under N₂, 20% CO₂, and 100% CO₂. The CV was recorded using 2.5 mM 6MCIId in DMF with 0.1 M supporting salt at a scan rate of −50 mV s⁻¹ with an initial cathodic sweeping direction at 298 K. (Grey: N₂; lighter colour: 20% CO₂; colour: 100% CO₂);

FIG. 36 is a summary of the peak potentials for CO₂ capture (filled symbols) and release (empty symbols) of isoindigos from FIG. 8 and FIG. 9. The theoretical energy costs for CO₂ capture and release were estimated based on the voltage gap (Eₚₑₐₖ release−Eₚₑₐₖ capture). The peak potentials for 02 reduction and oxidation are shown as grey dashed lines;

FIG. 37 shows the overall energy consumption of CO₂ concentration for each cycles in FIG. 6;

FIG. 38a, FIG. 38b, FIG. 38c, and FIG. 38d demonstrate the evaluation of the performance of 6MCIId at a higher percentage of CO₂ removal. FIG. 38a, CO₂ reading at the exit of the sorbent tank and voltage curve over 16 repeating capture/release cycles for approximately 120 h operation. FIG. 38b, CO₂ reading for selected capture/release cycles overlaid, with the cumulative amount of CO₂ captured/released in each cycle relative to the theoretical capacity. Lighter colours represent later cycles. Capture, rest, release, and rest steps are indicated by the shaded regions. For CO₂ capture, 6MCIId was reduced at 12 mA for 90 min followed by a 120 min rest. For CO₂ release, the adducts were oxidised at 12 mA to 0.05 mV followed by a approximately 120 min constant voltage hold, and finally rested for another 120 min. FIG. 38c, the voltage-capacity curve for the 2^{nd} capture/release cycle, indicating an early-stage energy consumption of 230.7 kJ per mol CO₂ concentrated. FIG. 38d, the CO₂ capacity utilisation efficiency (blue squares), release/capture efficiency (red crosses), and electrochemical efficiency (grey empty squares) of the system. The liquid sorbent was composed of 10 mL 0.2 M 6MCIId in DMF with 0.5 M NaTFSI as the supporting salt. The sorbent tank was filled with plastic beads and purged with 10% CO₂ balanced in N₂ at a flow rate of 1 sccm. On the opposite side, a Fc tank was used to balance the charge, which was filled with 20 mL 0.2 M Fc in DMF with 0.5 M NaTFSI as the supporting salt, 4 mM FcBF₄ to facilitate electro-reduction, and 10 mM 6MCIId to mitigate sorbent crossover;

FIG. 39a, FIG. 39b, FIG. 39c, FIG. 39d demonstrate the evaluation of the performance of 55DBIId in the flow-based EMCC prototype. FIG. 39a, CO₂ reading at the exit of the sorbent tank and voltage curve over repeating capture/release cycles for approximately 50 hours of operation. FIG. 39b, CO₂ reading of selected capture/release cycles overlaid, with the cumulative amount of CO₂ captured/released in each cycle relative to the theoretical capacity. Lighter colours represent later cycles. The capture, rest, release, and rest steps are indicated by the shaded regions. For CO₂ capture, 55DBIId was reduced at 10 mA for 60 min followed by a 60 min rest. For CO₂ release, the adducts were oxidised at 10 mA to 0.05 mV followed by an approximately 120 min constant voltage hold, and finally rested for another 60 min. FIG. 39c, the voltage-capacity curve for the 2^{nd} capture/release cycle, indicating an energy consumption of 135.2 kJ per mole of CO₂ concentrated. FIG. 39d, the CO₂ capacity utilisation efficiency (purple squares), release/capture efficiency (red crosses), and Coulombic efficiency (empty grey squares) of the system. The liquid sorbent was composed of 12 mL 75 mM 55DBIId slurry in DMAc with 0.25 M NaClO₄ as the supporting salt. On the opposite side, a Fc tank was used to balance the charge, which was filled with 30 mL 0.1 M Fc in DMAc with 0.25 M NaClO₄ as the supporting salt;

FIG. 40a, FIG. 40b, FIG. 40c, and FIG. 40d demonstrate the performance of 66DBIId in the flow-based EMCC prototype. FIG. 40a, CO₂ reading at the exit of the sorbent tank and voltage curve over repeating capture/release cycles for approximately 50 h operation. FIG. 40b, the CO₂ reading for selected capture/release cycles overlaid, with the cumulative amount of CO₂ captured/released in each cycle relative to the theoretical capacity. Lighter colours represent later cycles. The capture, rest, release, and rest steps are indicated by the shaded regions. For CO₂ capture, 66DBIId was reduced at 10 mA for 60 min followed by a 60 min rest. For CO₂ release, the adducts were oxidised at 10 mA to 0.05 mV followed by an approximately 120 min constant voltage hold, and finally rested for another 60 min. FIG. 40c, the voltage-capacity curve for the 2^{nd} capture/release cycle, indicating an early-stage energy consumption of 166.9 kJ per mol CO₂ concentrated. FIG. 40d, the CO₂ capacity utilisation efficiency (purple squares), release/capture efficiency (red crosses), and Coulombic efficiency (grey empty squares) of the system. The liquid sorbent was composed of 12 mL 75 mM 66DBIId slurry in DMAc with 0.25 M NaClO₄ as the supporting salt. On the opposite side, a Fc tank was used to balance the charge, which was filled with 30 mL 0.1 M Fc in DMAc with 0.25 M NaClO₄ as the supporting salt;

FIG. 41a, FIG. 41b, FIG. 41c, and FIG. 41d demonstrate the evaluation of the performance of 6B6AIIdSer in the flow-based EMCC prototype. FIG. 41a, CO₂ reading at the exit of the sorbent tank and voltage curve over 43 repeating capture/release cycles for approximately 190 h operation. FIG. 41b, CO₂ reading for selected capture/release cycles overlaid, with the cumulative amount of CO₂ captured/released in each cycle relative to the theoretical capacity. Lighter colours represent later cycles. The capture, rest, release, and rest steps are indicated by the shaded regions. For CO₂ capture, 6B6AIIdSer was reduced at 10 mA for 60 min followed by a 60 min rest. For CO₂ release, the adducts were oxidised at 10 mA to 0.05 mV followed by an approximately 80 min constant voltage hold, and finally rested for another 60 min. FIG. 41c, the voltage-capacity curve for the 2^{nd} capture/release cycle, indicating an early-stage energy consumption of 158.6 kJ per mol CO₂ concentrated. FIG. 41d, the CO₂ capacity utilisation efficiency (blue squares), release/capture efficiency (red crosses), and electrochemical efficiency (grey empty squares) of the system. The liquid sorbent was composed of 5 mL 0.2 M 6B6AIIdSer in DMF with 0.5 M NaTFSI as the supporting salt. On the opposite side, a Fc tank was used to balance the charge, which was filled with 10 mL 0.22 M Fc in DMF with 0.5 M NaTFSI as the supporting salt, and 10 mM 6B6AIIdSer to mitigate sorbent crossover;

FIG. 42a, FIG. 42b, FIG. 42c, and FIG. 42d show the NMR analysis of the sorbent tank after EMCC test of 66DBIId in FIG. 40. ¹H NMR of 66DBIId (up), crude ¹H NMR of sorbent tank after 11 capture-release cycles (middle), ¹H NMR of recovered 66DBIId from the sorbent tank;

FIG. 43a, FIG. 43b, FIG. 43c, and FIG. 43d demonstrate the evaluation of the performance of 6MCIId in simulated flue gas condition. FIG. 43a, CO₂ reading at the exit of the sorbent tank and voltage curve over 16 repeating capture/release cycles for approximately 90 h operation. FIG. 43b, CO₂ reading of selected capture/release cycles overlaid, with the cumulative amount of CO₂ captured/released in each cycle relative to the theoretical capacity. Lighter colours represent later cycles. Capture, rest, release, and rest steps are indicated by the shaded regions. For CO₂ capture, 6MCIId was reduced at 10 mA for 60 min followed by a 75 min rest. For CO₂ release, the adducts were oxidised at 10 mA to 0.05 mV followed by an approximately 120 min constant voltage hold, and finally rested for another 75 min. FIG. 43c, the voltage-capacity curve for the 2^{nd} capture/release cycle, indicating an energy consumption of 170.7 kJ per mole of CO₂ concentrated. FIG. 43d, the CO₂ capacity utilisation efficiency (green squares), release/capture efficiency (red crosses), and electrochemical efficiency (empty grey squares) of the system. The liquid sorbent was composed of 10 mL 0.1 M 6MCIId in DMF with 0.5 M NaTFSI as the supporting salt. The sorbent tank was filled with plastic beads and purged with 10% CO₂ and 3% O₂ balanced in N₂ at a flow rate of 2 sccm. On the opposite side, a Fc tank was used to balance the charge, which was filled with 20 mL 0.1 M Fe in DMF with 0.5 M NaTFSI as the supporting salt, 4 mM FcBF₄ to facilitate Fc oxidation, and 10 mM 6MCIId to mitigate sorbent crossover;

FIG. 44a, FIG. 44b, and FIG. 44c demonstrate the evaluation of CO₂ capture performance of 6MCIId in the flow-based EMCC prototype under 1% CO₂, 0.3% O₂. FIG. 44a, CO₂ reading on an IR-based sensor at the exit of the sorbent tank. FIG. 44b, Cumulative amount of CO₂ captured over time. FIG. 44c, the voltage-capacity curve of the capture process, indicating an early-stage energy consumption of 224.2 kJ per mol CO₂ captured. The area of the flow field and the carbon paper electrode are 25 cm². The liquid sorbent was composed of 10 mL 0.25 M 6MCIId in DMF with 0.5 M NaTFSI as the supporting salt. On the opposite side, a Fc tank was used to balance the charge, which was filled with 20 mL 0.25 M Fc in DMF with 0.5 M NaTFSI as the supporting salt, and 12.5 mM 6MCIId to mitigate sorbent crossover;

FIG. 45a, FIG. 45b, FIG. 45c, FIG. 45d, FIG. 45e, and FIG. 45f demonstrate the evaluation of CO₂ release performance of 6MCIId in the flow-based EMCC prototype under 100% CO₂. FIG. 45a and FIG. 45d, CO₂ mass flow rate on a mass flow metre at the exit of the sorbent tank. FIG. 45b and FIG. 45e, Cumulative amount of CO₂ captured and released over time, respectively. FIG. 45c and FIG. 45f, the voltage-capacity curve of the capture and release process, indicating an early-stage energy consumption of 143.7 kJ per mol CO₂ captured and 13.6 kJ per mol CO₂ released, respectively. The area of the flow field and the carbon paper electrode are 25 cm². The liquid sorbent was composed of 10 mL 0.25 M 6MCIId in DMF with 0.5 M NaTFSI as the supporting salt. On the opposite side, a Fc tank was used to balance the charge, which was filled with 20 mL 0.25 M Fc in DMF with 0.5 M NaTFSI as the supporting salt, and 12.5 mM 6MCIId to mitigate sorbent crossover.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a compound of formula (I):

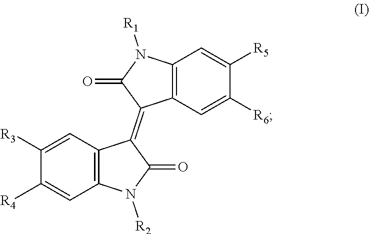

wherein:
R₁ and R₂ are each independently selected from H and branched or straightchain C₁-C₈ alkyl;
R₃, R₄, and R₆ are each independently selected from H, an electron withdrawing group, and an electron donating group;
R₅ is selected from H, an electron withdrawing group, an electron donating group, —COR_{y}, wherein R_{y} is an amino acid or a substituted amino acid, and —C(═O)—OR_{z}, wherein R_{z} is selected from H and branched or straightchain C₁-C₄ alkyl;
provided that at least one of R₁, R₂, R₃, R₄, R₅, and R₆ is not H; and
acceptable salts thereof.

In certain embodiments, the electron donating group is selected from alkoxide, hydroxyl, alkoxyl, amine, amide, ester, and alkyl. In certain aspects, the electron withdrawing group is selected from halogen, cyano, nitro, haloalkyl, ammonium, carbonyl, and sulfonyl.

In particular embodiments, R₁ and R₂ are each independently selected from H and branched or straightchain C₁-C₈ alkyl; R₃, R₄, and R₆ are each independently selected from H, branched or straightchain C₁-C₄ alkyl, halogen, C₁-C₄ alkoxyl, nitro, and —C(═O)—OR₇, wherein R₇ is branched or straightchain C₁-C₄ alkyl; R₅ is selected from H, branched or straightchain C₁-C₄ alkyl, halogen, C₁-C₄ alkoxyl, nitro, —C—OR₈, wherein R₈ is selected from an amino acid, a substituted amino acid, and —NHR₉, wherein R₉ is —CHR₁₀—C(═O)—O—Ru, wherein R₁₀ is H or —CH₂—OH, and Ru is branched or straightchain C₁-C₄ alkyl, and —C(═O)—OR₁₂, wherein R₁₂ is selected from H and branched or straightchain C₁-C₄ alkyl; provided that (i) at least one of R₁, R₂, R₃, R₄, R₅, and R₆ is not H, and (ii) R₄ and R₅ cannot both be Br at the same time if R₁, R₂, R₃, and R₆ are each H; and acceptable salts thereof.

As used herein, the term “alkyl” refers to a univalent group derived from an alkane by removal of a hydrogen atom from any carbon atom and having the chemical formula of —CₙH₂ₙ₊₁. The groups derived by removal of a hydrogen atom from a terminal carbon atom of unbranched alkanes form a subclass of normal alkyl (n-alkyl) groups H(CH₂)ₙ. The groups RCH₂, R₂CH (R≠H), and R₃C (R≠H) are primary, secondary, and tertiary alkyl groups, respectively.

An alkyl can be a straightchain (i.e., unbranched) or branched acyclic hydrocarbon having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons. In other embodiments, the alkyl can be a C₁-C₄ alkyl, including 1, 2, 3, and 4 carbons. In yet other embodiments, the alkyl can be a C₁-C₆ alkyl, including 1, 2, 3, 4, 5, and 6 carbons. In even yet other embodiments, the alkyl can be a C₁-C₈ alkyl, including 1, 2, 3, 4, 5, 6, 7, and 8 carbons.

“Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers to straight-chain alkyls. In other embodiments, “alkyl” refers to branched alkyls. In certain other embodiments, “alkyl” refers to straight-chain and/or branched alkyls. “Branched” refers to an alkyl group in which a lower alkyl group, such as methyl, ethyl, or propyl, is attached to a linear alkyl chain.

Representative alkyl groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, 2-ethylhexyl, n-heptyl, n-octyl, n-decyl, n-undecyl, and dodecyl.

Alkyl groups can optionally be substituted (a “substituted alkyl”) with one or more substituents, which can be the same or different. Such substituent groups include, but are not limited to, alkyl, substituted alkyl, cycloalkyl, halogen, acyl, carboxyl, oxo, aryl, substituted aryl, alkoxyl, hydroxyl, nitro, amino, alkylamino, dialkylamino, sulfate, cyano, and mercapto.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

As used herein, an “alkoxide” group is —O⁻.

As used herein, a “hydroxyl” group is —OH.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “amine” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

As used herein, an “amide” is —NH—C(═O)—R.

As used herein, an “ester” is —O—C(═O)—R.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

As used herein, “cyano” refers to —C≡N.

As used herein, “nitro” refers to —NO₂.

As used herein, “ammonium” refers to —N⁺R₃.

As used herein, “carbonyl” refers to —C(═O)—R.

The term “carboxyl” refers to the —COOH group. Such groups also are referred to herein as a “carboxylic acid” moiety.

As used herein, “sulfonyl” refers to —SO₃R.

As used herein, the term “amino acid” includes moieties having a carboxylic acid group and an amino group. The term amino acid thus includes both natural amino acids (including proteinogenic amino acids) and non-natural amino acids. The term “natural amino acid” also includes other amino acids that can be incorporated into proteins during translation (including pyrrolysine and selenocysteine). Additionally, the term “natural amino acid” also includes other amino acids, which are formed during intermediary metabolism, e.g., ornithine generated from arginine in the urea cycle. The natural amino acids are summarized immediately herein below:

Natural Amino Acids (Used For Protein Biosynthesis)

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

The natural or non-natural amino acid may be optionally substituted. In one embodiment, the amino acid is selected from proteinogenic amino acids. Proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine. The term amino acid includes alpha amino acids and beta amino acids, such as, but not limited to, beta alanine and 2-methyl beta alanine. The term amino acid also includes certain lactam analogues of natural amino acids, such as, but not limited to, pyroglutamine. The term amino acid also includes amino acids homologues including homocitrulline, homoarginine, homoserine, homotyrosine, homoproline and homophenylalanine.

The terminal portion of the amino acid residue or peptide may be in the form of the free acid i.e., terminating in a —COOH group or may be in a masked (protected) form, such as in the form of a carboxylate ester or carboxamide. In certain embodiments, the amino acid or peptide residue terminates with an amino group. In an embodiment, the residue terminates with a carboxylic acid group —COOH or an amino group —NH₂. In another embodiment, the residue terminates with a carboxamide group. In yet another embodiment, the residue terminates with a carboxylate ester.

As disclosed hereinabove, the term “amino acid” includes compounds having a —COOH group and an —NH₂group. A substituted amino acid includes an amino acid which has an amino group which is mono- or di-substituted. In particular embodiments, the amino group may be mono-substituted. (A proteinogenic amino acid may be substituted at another site from its amino group to form an amino acid which is a substituted proteinogenic amino acid). The term substituted amino acid thus includes N-substituted metabolites of the natural amino acids including, but not limited to, N-acetyl cysteine, N-acetyl serine, and N-acetyl threonine. The term substituted amino acid also includes acetyl-L-leucylglycine.

For example, the term “N-substituted amino acid” includes N-alkyl amino acids (e.g., C₁₋₆ N-alkyl amino acids, such as sarcosine, N-methyl-alanine, N-methyl-glutamic acid and N-tert-butylglycine), which can include C₁₋₆ N-substituted alkyl amino acids (e.g., N-(carboxy alkyl) amino acids (e.g., N-(carboxymethyl)amino acids) and N-methylcycloalkyl amino acids (e.g., N-methylcyclopropyl amino acids)); N,N-di-alkyl amino acids (e.g., N,N-di-C₁₋₆ alkyl amino acids (e.g., N,N-dimethyl amino acid)); N,N,N-tri-alkyl amino acids (e.g., N,N,N-tri-C₁₋₆ alkyl amino acids (e.g., N,N,N-trimethyl amino acid)); N-acyl amino acids (e.g., C₁₋₆ N-acyl amino acid); N-aryl amino acids (e.g., N-phenyl amino acids, such as N-phenylglycine); N-amidinyl amino acids (e.g., an N-amidine amino acid, i.e., an amino acid in which an amine group is replaced by a guanidino group).

The term “amino acid” also includes amino acid alkyl esters (e.g., amino acid C₁₋₆ alkyl esters); and amino acid aryl esters (e.g., amino acid phenyl esters).

For amino acids having a hydroxy group present on the side chain, the term “amino acid” also includes O-alkyl amino acids (e.g., C₁₋₆ O-alkyl amino acid ethers); O-aryl amino acids (e.g., O-phenyl amino acid ethers); O-acyl amino acid esters; and O-carbamoyl amino acids.

For amino acids having a thiol group present on the side chain, the term “amino acid” also includes S-alkyl amino acids (e.g., C₁₋₆ S-alkyl amino acids, such as S-methyl methionine, which can include C₁₋₆ S-substituted alkyl amino acids and S-methylcycloalkyl amino acids (e.g., S-methylcyclopropyl amino acids)); S-acyl amino acids (e.g., a C₁₋₆ S-acyl amino acid); S-aryl amino acid (e.g., a S-phenyl amino acid); a sulfoxide analogue of a sulfur-containing amino acid (e.g., methionine sulfoxide) or a sulfoxide analogue of an S-alkyl amino acid (e.g., S-methyl cysteine sulfoxide) or an S-aryl amino acid.

In other words, the presently disclosed subject matter also includes derivatives of natural amino acids, such as those mentioned above which have been functionalized by simple synthetic transformations known in the art (e.g., as described in “Protective Groups in Organic Synthesis” by T W Greene and P G M Wuts, John Wiley & Sons Inc. (1999)), and references therein.

Examples of non-proteinogenic amino acids include, but are not limited to: citrulline, hydroxyproline, 4-hydroxyproline, β-hydroxyvaline, ornithine, β-amino alanine, albizziin, 4-amino-phenylalanine, biphenylalanine, 4-nitro-phenylalanine, 4-fluoro-phenylalanine, 2,3,4,5,6-pentafluoro-phenylalanine, norleucine, cyclohexylalanine, α-aminoisobutyric acid, α-aminobutyric acid, α-aminoisobutyric acid, 2-aminoisobutyric acid, 2-aminoindane-2-carboxylic acid, selenomethionine, lanthionine, dehydroalanine, 7-amino butyric acid, naphthylalanine, aminohexanoic acid, pipecolic acid, 2,3-diaminoproprionic acid, tetrahydroisoquinoline-3-carboxylic acid, tert-leucine, tert-butylalanine, cyclopropylglycine, cyclohexylglycine, 4-aminopiperidine-4-carboxylic acid, diethylglycine, dipropylglycine and derivatives thereof wherein the amine nitrogen has been mono- or di-alkylated.

In certain embodiments, R₁ and R₂ are each H. In other embodiments, R₁ and R₂ are each branched or straightchain C₁-C₈ alkyl. In particular embodiments, R₁ and R₂ are each —CH₂CH₂—CH₃ or —CH₂CH(CH₂CH₃)(CH₂CH₂CH₂CH₃).

In certain embodiments, one or more of R₃, R₄, R₅, and R₆ is selected from branched or straightchain C₁-C₄ alkyl, C₁-C₄ alkoxyl, halogen, nitro, and —C(═O)—OR₁₂, wherein R₁₂ is H or branched or straightchain C₁-C₄ alkyl.

In certain embodiments, (i) R₃ and R₆ are each the same and are not H; (ii) R₄ and R₅ are the each same and are not H; or (iii) R₃, R₄, R₅, and R₆ are each the same and are not H. In such embodiments, the compounds of formula (I) are referred to as symmetric isoindigos.

In certain embodiments, (i) R₃ is halogen and R₄, R₅, and R₆ are each H; (ii) R₄ is halogen and R₃, R₅, and R₆ are each H; (iii) R₅ is —C(═O)—OR₁₂, wherein R₁₂ is H or branched or straightchain C₁-C₄ alkyl, R₃ and R₆ are each H, and R₄ is H or halogen; or (iv) R₆ is nitro and R₅ is H, R₃ is H or C₁-C₄ alkoxyl, and R₄ is H or —C(═O)—OR₁₂, wherein R₁₂ is H or branched or straightchain C₁-C₄ alkyl. In such embodiments, the compounds of formula (I) are referred to as nonsymmetric isoindigos.

In particular embodiments, the compound of formula (I) is a symmetric isoindigo and is selected from:

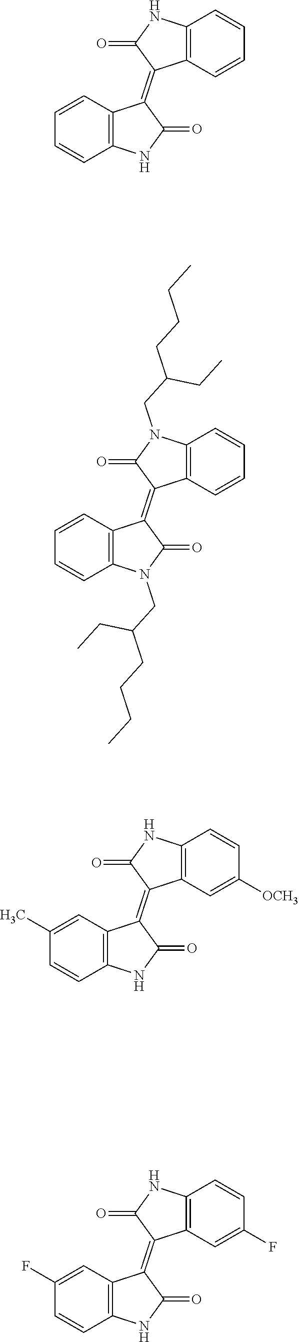 [SMILES: O=C1Nc2ccccc2/C1=C1\C(=O)Nc2ccccc21 ; CCCCC(CC)CN1C(=O)/C(=C2/C(=O)N(CC(CC)CCCC)c3ccccc32)c2ccccc21 ; COc1ccc2c(c1)/C(=C1\C(=O)Nc3ccc(C)cc31)C(=O)N2 ; O=C1Nc2ccc(F)cc2/C1=C1\C(=O)Nc2ccc(F)cc21]

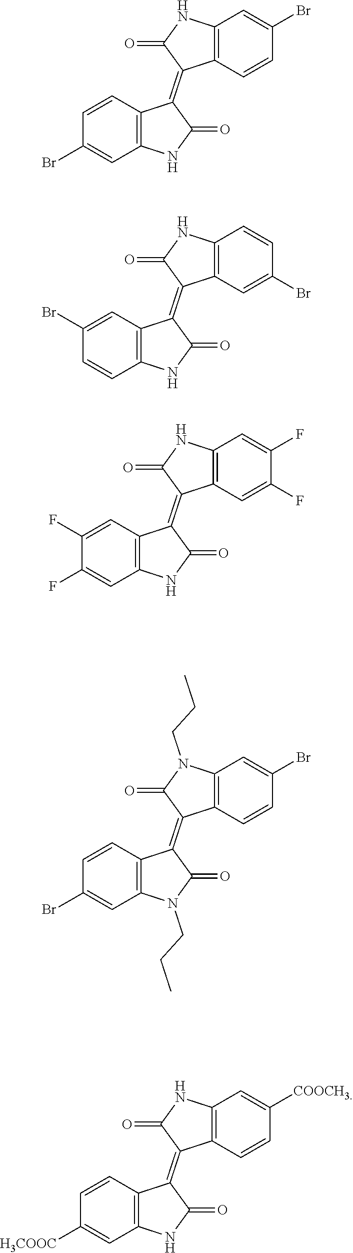

In other embodiments, the compound of formula (I) is a nonsymmetric isoindigo and is selected from:

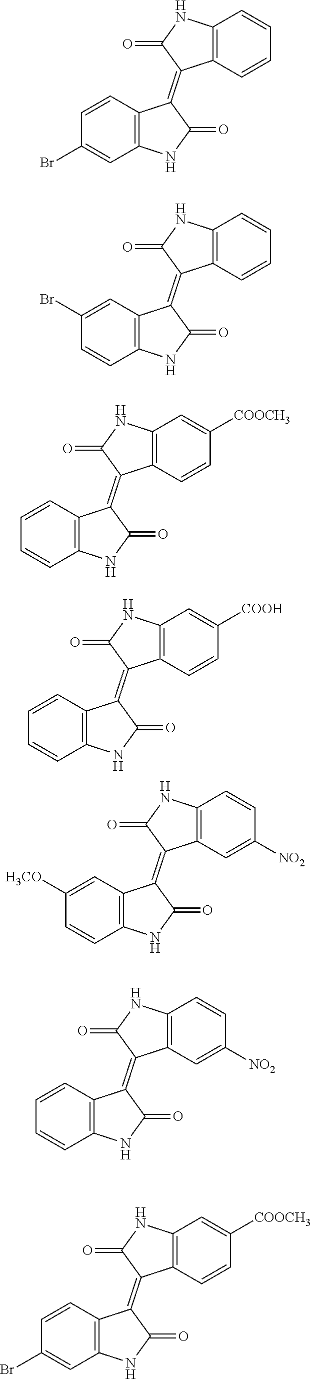

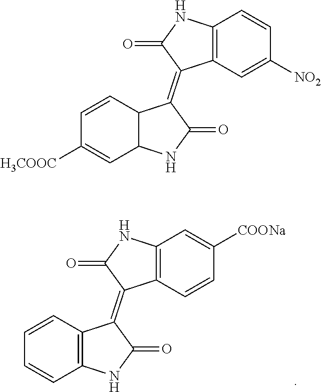

In certain embodiments, the compound of formula (I) comprises a compound of formula (Ia):

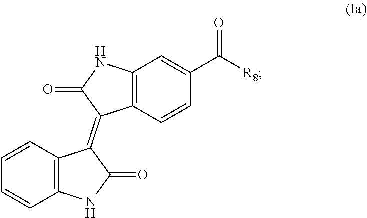

wherein: R₈ is —NHR₉, wherein R₉ is —CHR₁₀—C(═O)—O—R₁₁, wherein R₁₀ is H or —CH₂—OH, and R₁₁ is branched or straightchain C₁-C₄ alkyl. In particular embodiments, the compound of formula (Ia) is selected from:

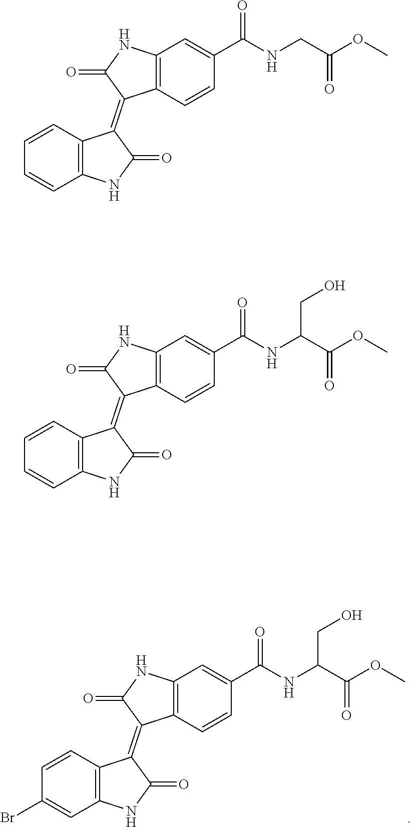 [SMILES: COC(=O)CNC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2cc(Br)ccc21]

In certain embodiments, the presently disclosed subject matter provides a sorbent material comprising a compound of formula (I), or in certain embodiments, a compound of formula (Ia).

In other embodiments, the presently disclosed subject matter provides a process for capturing CO₂ from a gas sample, the process comprising contacting the gas sample with a compound of formula (I), or a sorbent material comprising a compound of formula (I).

In certain embodiments, the process comprises an electrochemically mediated carbon capture (EMCC) process. In particular embodiments, the process comprises (i) electro-reduction of the sorbent to form an adduct with CO₂; and (ii) oxidising the adduct to liberate CO₂ regenerate the sorbent.

In certain embodiments, the gas sample is selected from ambient air, i.e., direct air capture, ventilated air, and a stream of gas, e.g., flue gas or other emissions from a point source, such as an industrial source, such as a chemical plant, petroleum production facility, a cement plant, power generation, and transportation, and from enclosed spaces, for example, in buildings and in the cabin space of cars, trains, airplanes, spacecraft, submarines, and the like.

In certain embodiments, the process is a fixed-bed process or a flow-based process.

In other embodiments, the presently disclosed subject matter provides a system for separating carbon dioxide from ambient air or a stream of gases, the system comprising the compound of formula (I) or a sorbent material comprising a compound of formula (I).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±20%, in some embodiments ±15%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner.

### Example 1

### Redox-Tunable Isoindigos for Electrochemically Mediated Carbon Capture

### 1.1 Overview

The presently disclosed subject matter demonstrates a new design paradigm for EMCC sorbents, which breaks the undesirable scaling relationship by leveraging intramolecular hydrogen bonding in isoindigo derivatives. The redox potentials of isoindigos can be anodically shifted by more than 350 mV to impart sorbents with high oxygen stability without compromising CO₂ binding, culminating in an EMCC system with minimised parasitic reactions. With a vast synthetic space presented, the presently disclosed subject matter provides a generalisable strategy to finetune interactions between redox-active organic molecules and CO₂, thereby addressing a longstanding challenge in developing effective carbon capture methods driven by non-conventional stimuli.

### 1.2 Background

Carbon capture from stationary emitters or directly from the ambient environment, followed by sequestration or utilisation, is critical to mitigating climate change. Mac Dowell et al., 2017; Deutz and Bardow, 2021; Bui et al., 2018. Wet chemical scrubbing methods for carbon dioxide (CO₂) separation known in the art, however, are technically and economically challenged by various inherent limitations, including high energy consumption for sorbent regeneration, thermal degradation, complexity in heat integration when retrofitting existing infrastructures, process equipment corrosion, and fugitive emission of volatile toxic sorbents to the environment. Chu, 2009; Sanz-Pérez et al., 2016. Alternatively, electrochemically mediated carbon capture (EMCC) has emerged as a promising technology. Sharifian et al., 2021; Liu et al., 2022; Rheinhardt et al., 2017; Renfrew et al., 2020; Barlow et al., 2022; Diederichsen et al., 2022.

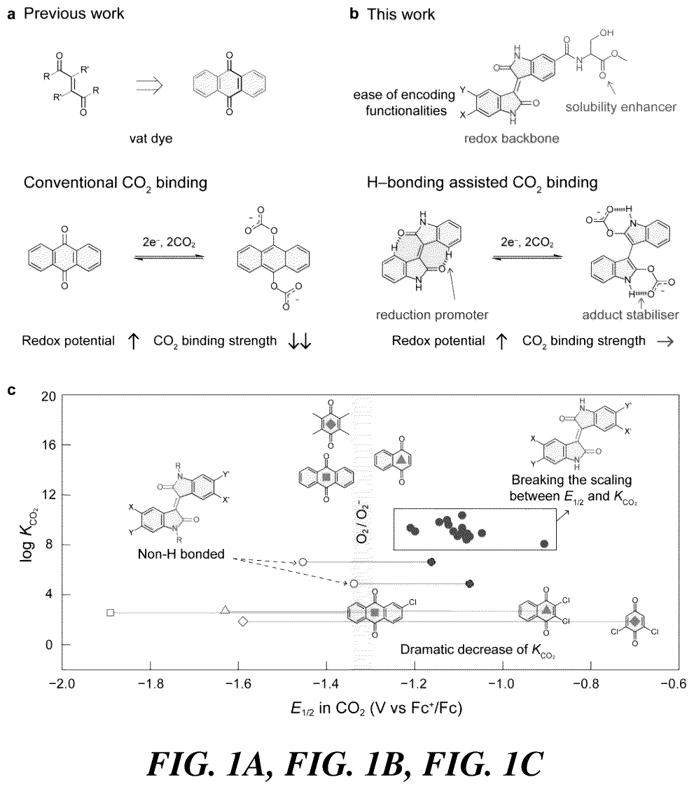

In EMCC, reversible CO₂ capture and release is modulated by switching electrochemical potentials. Therefore, they can be operated isothermally at ambient pressure, powered by renewable energy sources, and modularly designed to accommodate the multiscale nature of carbon capture needs. Among the EMCC mechanisms explored to date, one popular strategy is to use redox-active organic compounds as CO₂ carriers (redox-tunable Lewis bases), with quinones being the most studied class of molecules. Bell et al., 1988; Mizen and Wrighton, 1989; Nagaoka et al., 1992. Electro-reduction of these molecules generates nucleophiles that form adducts with electrophilic CO₂, which can be later oxidised to liberate pure CO₂ while regenerating the sorbents (FIG. 1a).

The past two decades have witnessed steady research progress in developing EMCC processes using redox-tunable sorbents. Rheinhardt et al., 2017; Barlow et al., 2022; Rahimi et al., 2022; Scovazzo et al., 2003; Gurkan et al., 2015; Voskian et al., 2019; Diederichsen et al., 2021; Li et al., 2022. Several bench-scale prototypes have been demonstrated for fixed-bed and flow-based CO₂ separations attributed to materials and device-level engineering efforts. Gurkan et al., 2015; Voskian and Hatton, 2019; Diederichsen et al., 2021. In contrast, molecular-level design principles for precise sorbent property tuning remain largely unestablished beyond the simplistic method of structural substitution with electron-donating and withdrawing groups, despite their central role in EMCC.

The lack of reliable sorbent chemistry has intrinsically hindered existing EMCC processes. A practical EMCC system usually requires chemical modification of redox-tunable sorbents to improve key properties including, but not limited to, solubility, processability, and stability against impurities. For example, anodically shifting the redox potential will enhance the robustness of activated sorbent against molecular oxygen (O₂), a common gas stream impurity, for improved carbon capture efficiency. Nevertheless, the CO₂ binding affinity of existing redox-tunable Lewis base sorbents, such as quinones, is highly susceptible to chemical modifications, which decreases dramatically as the redox potential shifts anodically. Simeon et al., 2022; Barlow and Yang, 2022; Bui et al., 2022.

The seminal work from W. L. Bell et al. introduced a method to calculate the CO₂ binding constant (K_{CO}_{₂}) of activated sorbent, which is widely adopted later to evaluate the CO₂ binding strength. Bell et al., 1988. As an example, anthraquinone exhibits a two-electron-transfer half-wave potential (E_{1/2}) of −1.4 V vs. ferrocenium/ferrocene (Fc⁺/Fc) in N,N-dimethylformamide (DMF) under CO₂ and a log K_{CO}_{₂} of approximately 13.4 (FIG. 1c). The installation of electron-withdrawing groups (EWGs), such as one chloro group at 2-position, can anodically shift E_{1/2} to −1.25 V vs. Fc⁺/Fc, yet the log K_{CO}_{₂}decreased substantially to 2.73 (FIG. 1c and Table 2). Simeon et al., 2022.

A minimum log K_{CO}_{₂} of approximately 3.0 in DMF is required to attain a practical efficiency for point source carbon capture (10% CO₂), and the log K_{CO}_{₂} must be greater than approximately 5.5 for atmospheric CO₂ concentration (400 ppm). Bell et al., 1988.

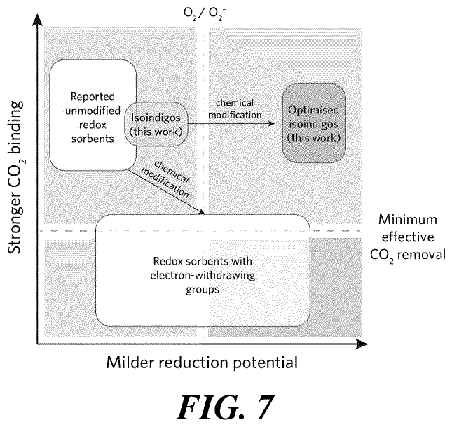

Importantly, it is challenging to overcome the coupling between E_{1/2} and log K_{CO}_{₂} as it is dictated by the fundamental principle in chemistry that electron deficiency facilitates reduction but in return weakens nucleophilicity. Hansch et al., 1991. Therefore, to fulfil the practical requirements of EMCC, it is pivotal to develop new classes of redox-tunable sorbents that can break the linear free-energy relationship between redox potential and CO₂ binding strength to enable aerobic stability and high CO₂ capacity (FIG. 7).

### 1.3 Scope

The presently disclosed subject matter presents a new class of redox-tunable CO₂ carriers based on isoindigo compounds, which can successfully overcome the undesirable coupling between redox potential and CO₂ binding strength (FIG. 1b and FIG. 1c). With a molecular library of 21 examples and a combined experimental and computational effort, the presently disclosed subject matter shows that the α,β-unsaturated 1,4-diketone in isoindigos plays the role of redox backbone for CO₂ binding and the amide groups act as extra docking sites for CO₂ complexation via intramolecular hydrogen bonding. This unique bifunctional structural design allows a wide range of chemical modifications to independently optimise key sorbent properties without sacrificing their abilities for CO₂ binding. The isoindigo family culminates in an EMCC system that can operate at mild potentials (around −1 V vs. Fc⁺/Fc) with log K_{CO}_{₂} maintained at approximately 9. This value is five orders of magnitude higher than tetrachloroquinone with alcohol additives, Barlow and Yang, 2022, which is the state-of-the-art sorbent chemistry with an attempt to break the linear free-energy relationship.

Flow-based separation prototypes also have been demonstrated to evaluate the EMCC performance of the isoindigo sorbents, which can achieve CO₂ capacity utilisation efficiencies up to approximately 80% and energy consumptions as low as 127.3 kJ mol⁻¹ per CO₂ capture/release cycle. With intrinsic 02 stability, high structural tunability, and synthetic feasibility, isoindigos are promising to serve as the next-generation EMCC sorbents. Moreover, the presently disclosed subject matter demonstrates a generalisable strategy to overcome the intrinsic linear free-energy limits in redox-active organic species that can be broadly applied to EMCC and beyond.

### 1.4 Results

1.4.1 Redox-Tunable CO₂ Absorption of Isoindigo Isoindigo and its derivatives have been extensively utilised as core building blocks in organic semiconductors, Wang et al., 2014; Bogdanov and Mironov, 2021; Kiss et al., 2021; Wei et al., 2021, but have rarely been explored as redox molecules for organic electrodes. Without wishing to be bound to any one particular theory, it is thought that isoindigos bearing α,β-unsaturated 1,4-diketone functionalities to be redox-active and can complex with CO₂ at the oxygen centres in the reduced state. A series of electrochemical experiments was conducted to validate the redox-driven interaction between isoindigo (IId) and CO₂.

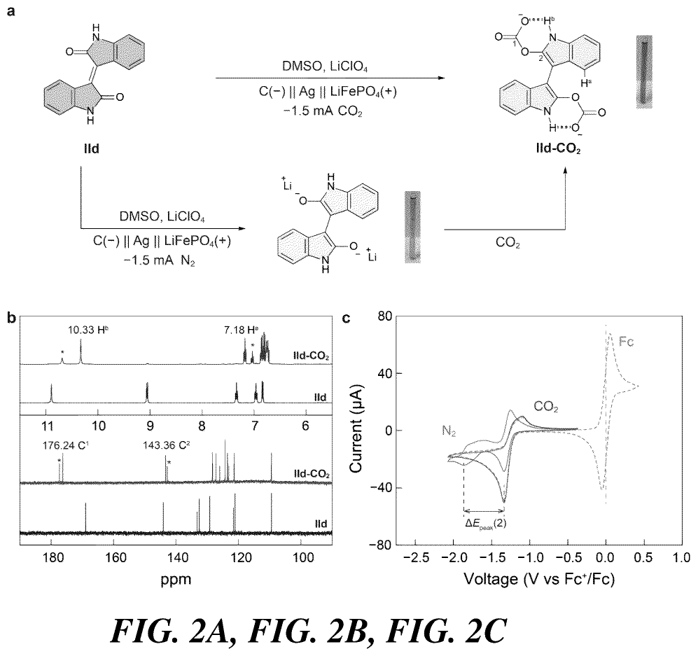

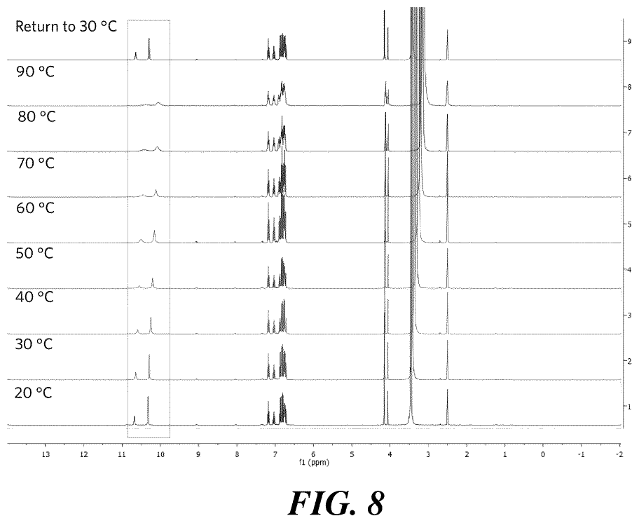

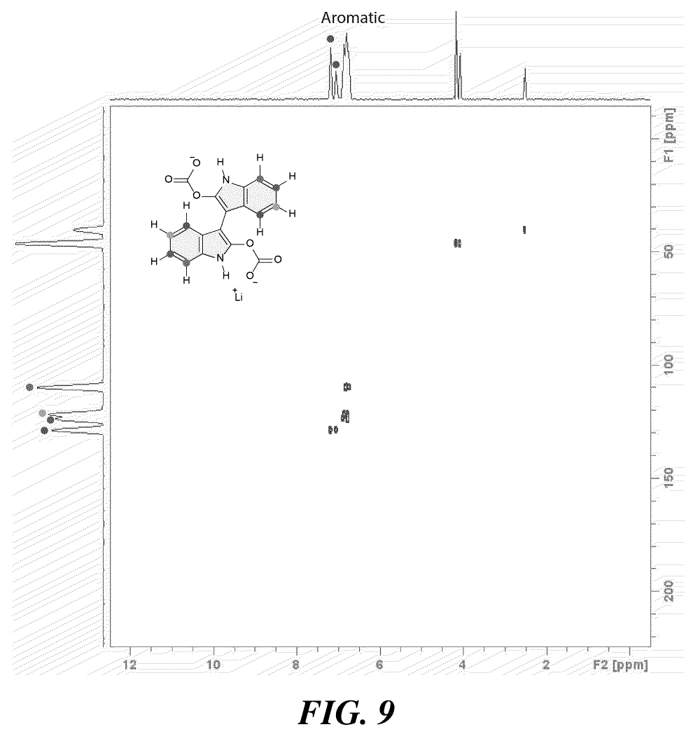

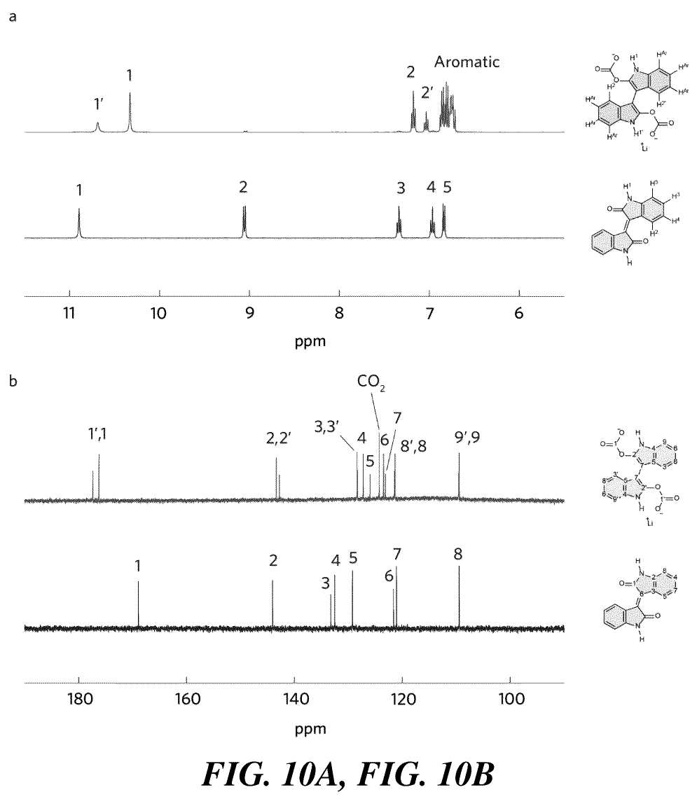

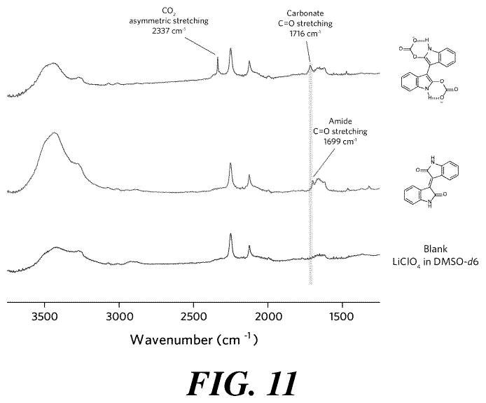

First, we performed bulk electrolysis of IId using 0.25 M lithium perchlorate (LiClO₄) in dimethyl sulfoxide (DMSO) as the supporting electrolyte under N₂ or CO₂ atmosphere (FIG. 2a). Under N₂, the dark dispersion of IId turned greenish at the beginning of the reduction and became a clear yellow solution when the reaction was completed. This observation suggests the formation of the bisindolidenolate intermediate, which is expected to absorb CO₂ as a Lewis base. Interestingly, the yellow solution quickly turned red when purged with CO₂, implying the formation of the IId-CO₂ adduct. ¹H and ¹³C nuclear magnetic resonance (NMR) spectra of the crude solution confirm the full conversion of IId into IId-CO₂ with negligible side products (FIG. 2b). Characteristic peaks for the carbonate carbon were observed at between 176-177 pm in ¹³C NMR. Further, the proton on the lactam N shifts upfield to 10.33 ppm in IId-CO₂ compared to that of 10.89 ppm in IId, strongly evidencing the formation of intramolecular hydrogen bonding with complexed CO₂ (highlighted in red in FIG. 2a). The NMR spectra exhibit two types of amide hydrogens and carbonate carbons, which is probably due to the rotational isomerisation of IId-CO₂ (FIG. 8). The presence of intramolecular hydrogen bonding in IId-CO₂ was further verified by variable-temperature (VT) ¹H NMR, 2D ¹H-¹³C Heteronuclear Single Quantum Coherence (HSQC) NMR, and Fourier transform infrared (FT-IR) experiments (FIGS. 9-11, see Section 1.7 for detailed analysis). The full NMR peak assignment of IId and IId-CO₂ is given in FIG. 8.

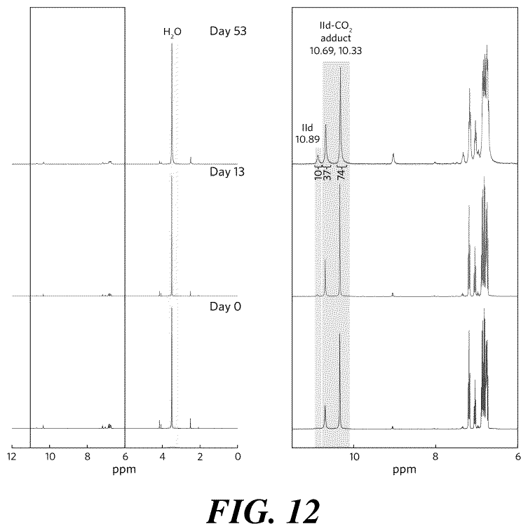

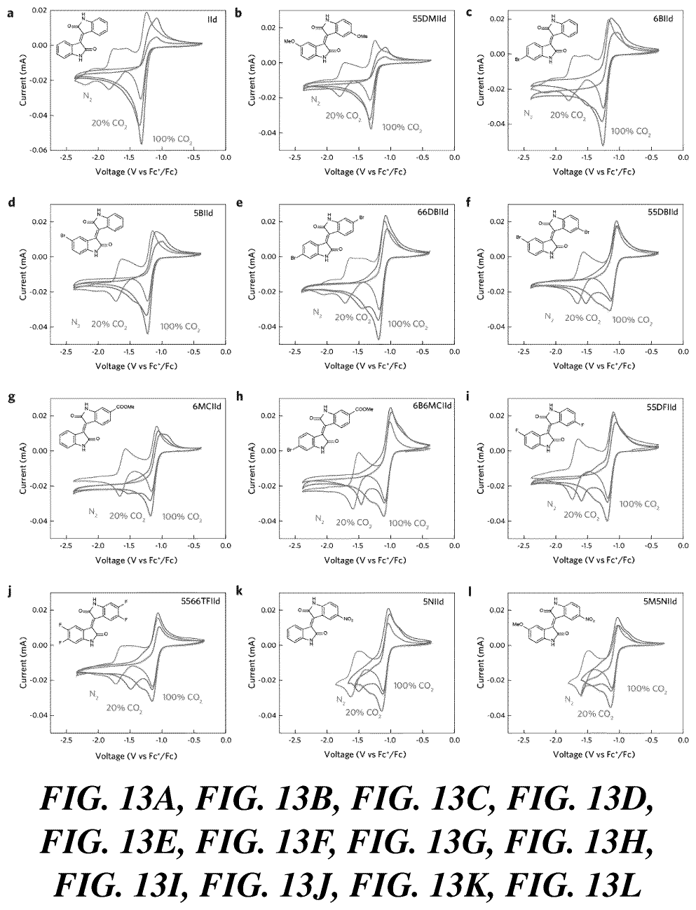

Since the discovery of the EMCC mechanism using quinones in 1988, Bell et al., 1988; Mizen and Wrighton, 1989, to our best knowledge, the widely accepted electrochemically generated quinone-CO₂ carbonate adduct is still a proposed structure and has not been confirmed in an EMCC process by non-ambiguous characterisations. This lack of confirmation is probably due to the poor stability of the adducts and the transient bonding nature between the reduced sorbent and CO₂. On the contrary, crude NMR spectra suggest bulk electro-reduction of IId under CO₂ can yield the proposed adduct with full conversion (IId was fully consumed), indicating the high selectivity of this chemistry and the sufficient stability of the adduct, which we postulate to be the result of intramolecular hydrogen bonding. Noticeably, the IId-CO₂ solution obtained from bulk electrolysis can be stably stored in ambient air with high water content. After 53 days, less than 9 mol % of IId-CO₂ oxidised back to the neutral IId form (FIG. 12).

To confirm the redox activity of IId, we measured its cyclic voltammetry (CV) using 0.1 M tetrabutylammonium hexafluorophosphate (NBu₄PF₆) in DMF as the supporting electrolyte (FIG. 2c). Under an inert N₂ atmosphere, IId exhibits two major redox waves typical to stepwise two-electron transfer. Like quinoid species, Mizen and Wrighton, 1989, the two electron transfer steps correspond to the formation of anionic radicals (IId·−) and dianions (IId2−), respectively. The two reduction peaks under N₂ emerge into one in the presence of CO₂ with a nearly doubled peak current, indicating chemical interactions between reduced IId and CO₂. This behaviour is analogous to the other redox-tunable CO₂ sorbents reported previously based on oxygen or nitrogen binding centres, whose reaction with CO₂ proceeds via an ECEC mechanism (E, electron transfer; C, chemical reaction). Bell et al., 1988; Mizen and Wrighton, 1989; Li et al., 2022.

As a Lewis acid, CO₂ can withdraw the electron density from IId·− to promote the second electron transfer, giving rise to an anodically shifted second reduction wave. Further, the oxidation peak also shifts anodically and becomes quasi-reversible, corroborating the formation of the IId-CO₂ adduct, which requires more energy for CO₂ desorption.

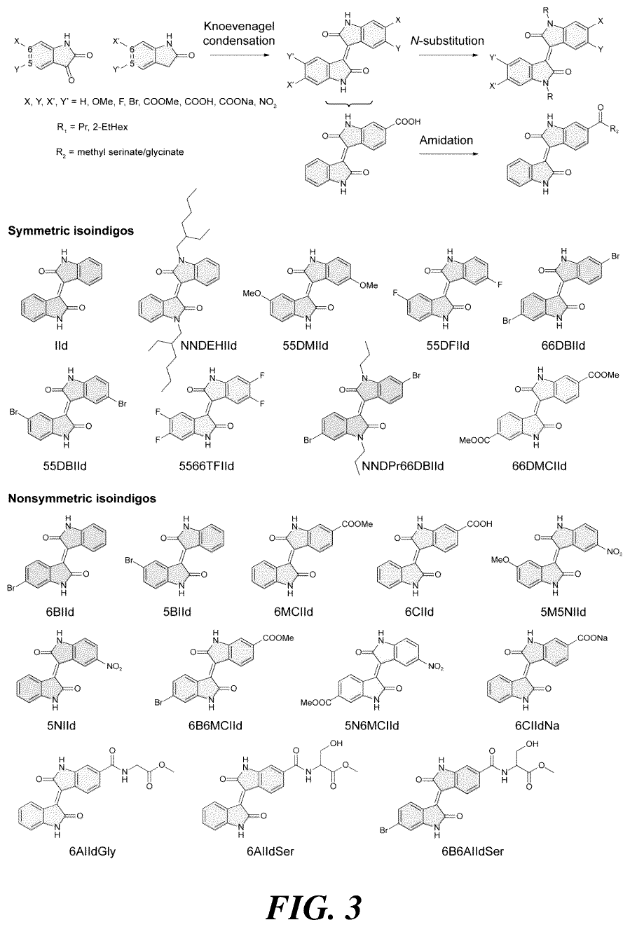

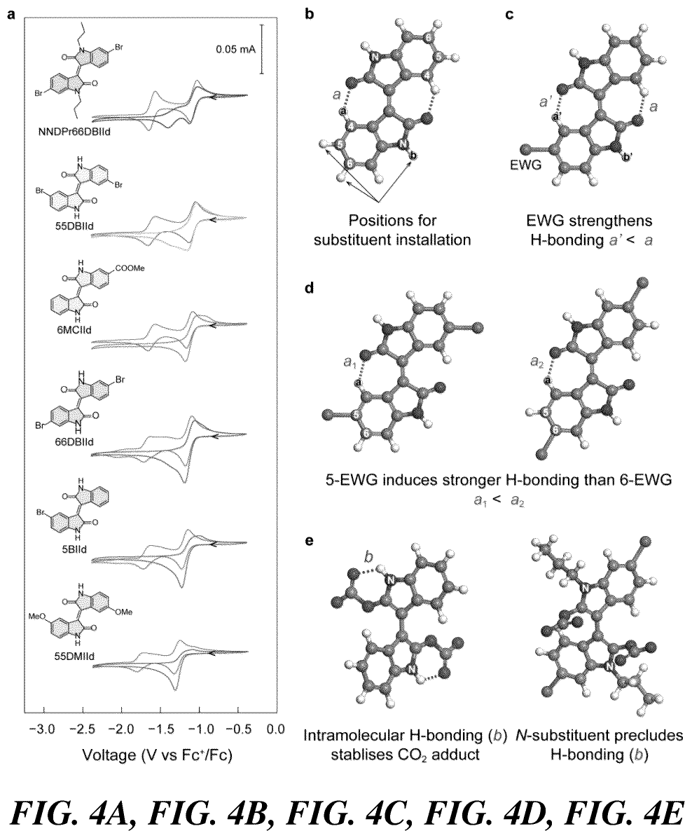

This preliminary finding encouraged us to expand the chemical scope of isoindigos and search for more qualified EMCC compounds. Isoindigos can be synthesised through the condensation reaction between 2-oxindoles and isatins. This reaction allows the modular design of redox sorbents, where the two building units can be modified separately and integrated into isoindigos in the last step, thereby addressing the synthetic barriers when engineering EMCC sorbents (Section 1.8). The presently disclosed subject matter, in part, demonstrates the structural modification of isoindigo at the 5, 6, and N-positions with 21 examples (FIG. 3) and examines in detail the interplay between substituent groups and hydrogen bonding on the redox and CO₂ binding behaviours of isoindigos to establish the underlying structure-property relationships. The main conclusions are summarised in FIG. 4 and will be discussed in detail in the following sections.

### 1.4.2 Tuning the Redox Potential of Isoindigos

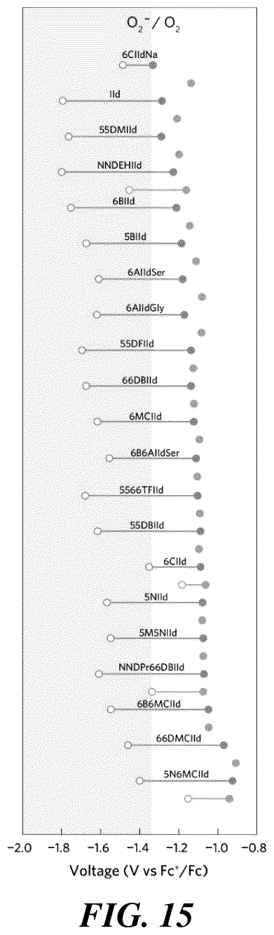

The redox behaviours of derivatised isoindigos were examined under N₂ via CV. All of the isoindigos tested displayed redox couples typical to stepwise two-electron transfer, underscoring the good electrochemical reversibility of these molecules (FIG. 4a, FIGS. 13-15).

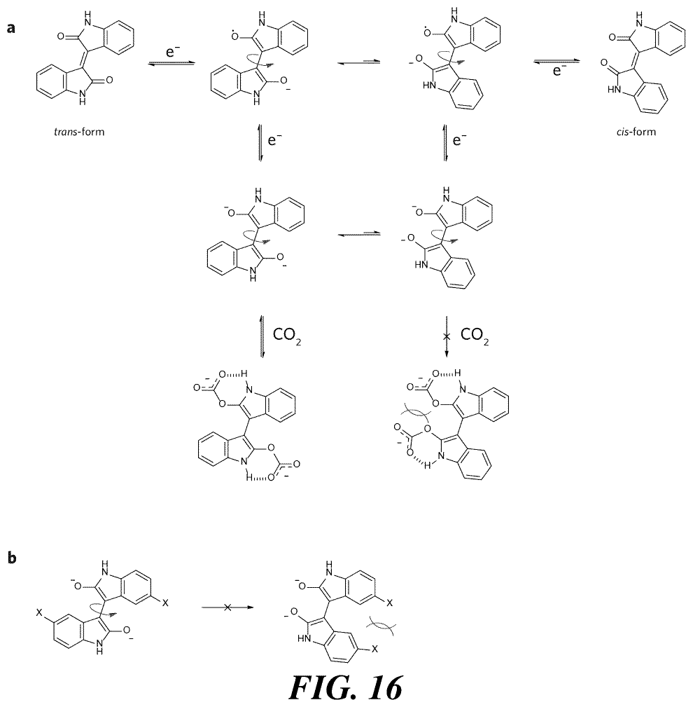

Among the 21 examples of isoindigos, only IId, 6BIId, and 66DBIId exhibit less well-defined shapes for the second redox wave. This result is probably due to the rotational isomerisation of the one-electron reduced isoindigo radical anions. As shown in FIG. 16, the C═C bond connecting the two oxindole rings becomes a single bond in the radical anion, allowing free rotation of the two rings. This rotation about the single bond creates rotational isomers and gives rise to shoulder peaks in the second redox process. Adding strong EWGs or substitution groups at the 5-position of isoindigo can create dipole-dipole repulsion or steric hindrance, inhibiting rotational isomerisation and resulting in a more reversible second redox process. Similarly, adding CO₂ onto isoindigos also creates dipole and steric hindrance, impeding the rotational isomerisation and leading to more defined CV curves.

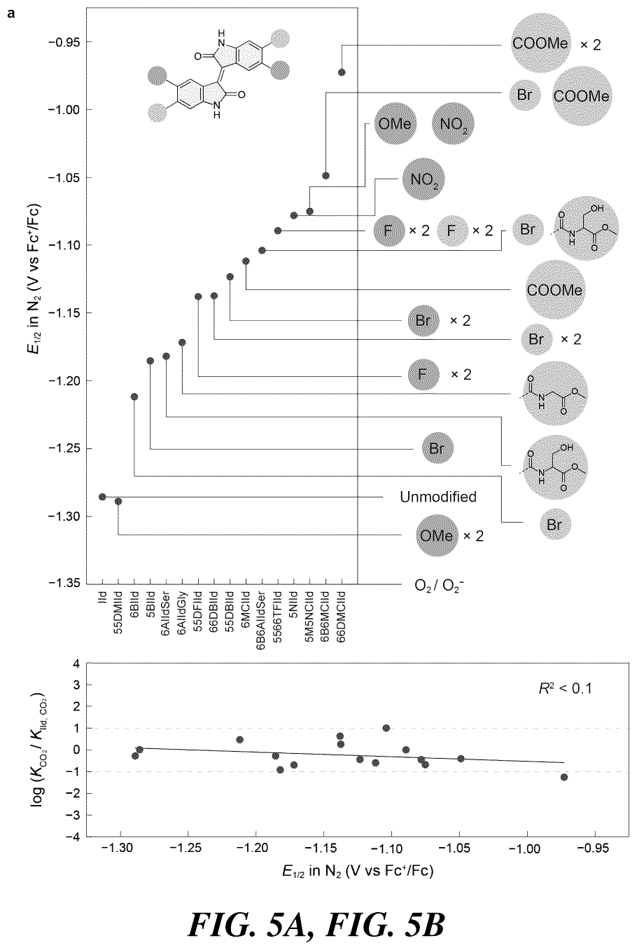

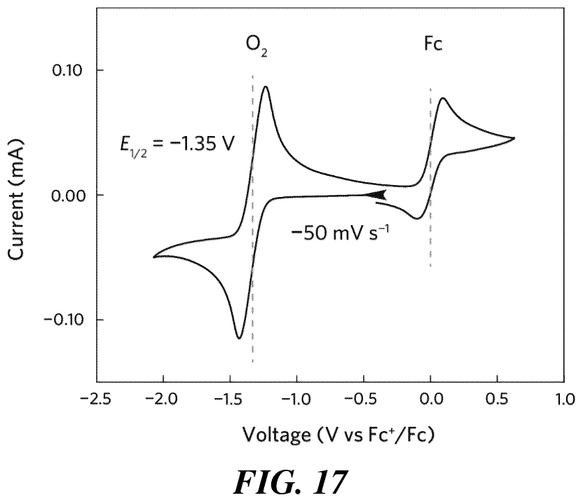

Ideal EMCC sorbents shall have redox potentials more positive than the oxygen reduction potential (−1.35 V vs. Fc⁺/Fc in DMF, FIG. 17) to minimize sorbent sensitivity towards O₂. With stronger or increasing numbers of EWG introduced to the isoindigo rings, the redox potential exhibits an increasing anodic shift in the sequence of —F, —Br, —COOMe, —CONHR, and —NO₂ substituent groups from mono- to tetra-substitution (FIG. 5a and Table 3). Without wishing to be bound to any one particular theory, through a close examination of the structure-property relationship, it is thought that the anodic shift is attributed to not only the commonly expected electronic state tuning from the EWG substituents but also the intramolecular hydrogen bonding effect (a shown in FIG. 4b and FIG. 4c). This hypothesis is because hydrogen bonding can decrease the electron density of the redox-active oxygen centre to facilitate reduction. The downfield shift of proton at 4-position (Ha) in isoindigos with EWG substituents suggests the formation of stronger hydrogen bonding (a) (Table 1 and Table 3).

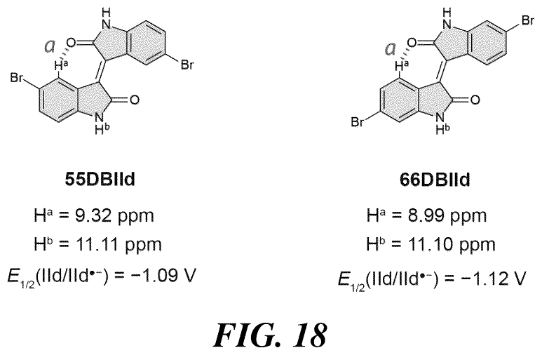

To verify the above hypothesis, we show that EWG substituent at 5-position is more effective in facilitating electro-reduction than that at 6-position. This increase in effectiveness arises because the former is in the ortho-position of Ha and more effective in pulling away the electron density, thereby inducing a stronger hydrogen bonding (FIG. 4d). For instance, 55DBIId and 66DBIId exhibit very close 1H NMR peaks for Hb (11.11 and 11.10 ppm, FIG. 18), suggesting similar degrees of electron deficiency in these two molecules. In contrast, the chemical shift of Ha is 9.32 and 8.99 ppm for 55DBIId and 66DBIId, respectively, clearly indicating a stronger hydrogen bonding (a) in the 5-substituted species. Therefore, E_{1/2}(IId/IId·−) of 55DBIId (−1.09 V vs. Fc⁺/Fc) is more positive compared to 66DBIId (−1.12 V vs. Fc⁺/Fc). This trend is consistent for all examples in our isoindigo family (Table 3, e.g., 5BIId vs. 6BIId and 6MCIId vs. 5NIId vs. 5N6MCIId) and is further confirmed with DFT calculations (details vide post).

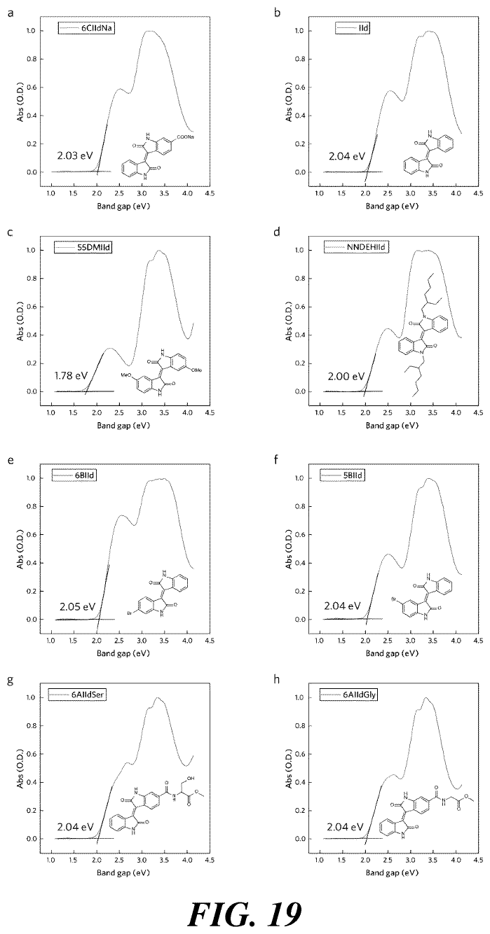

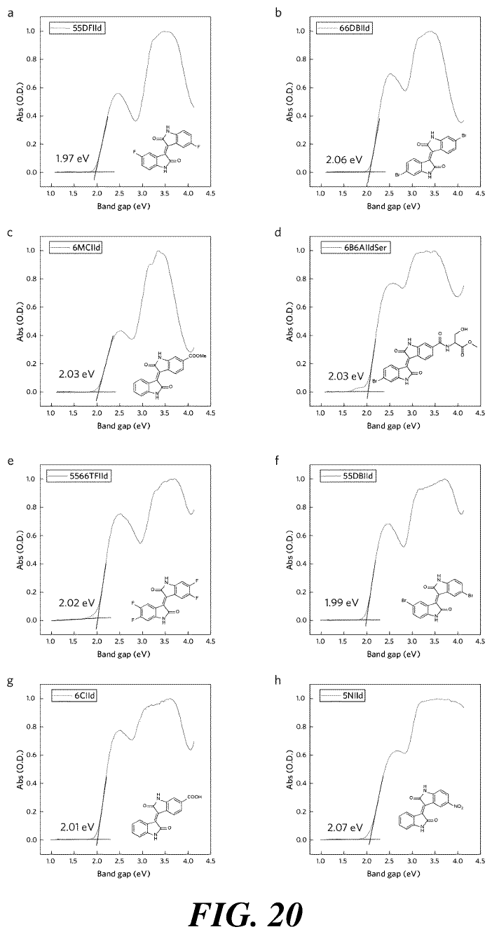

Counterintuitively, adding electron-donating groups (EDGs), such as methoxy shows a negligible influence on E_{1/2}(IId/IId·−), as evidenced by IId vs. 55DMIId (both show E_{1/2} of −1.29 V vs. Fc⁺/Fc, Table 1), and 5NIId vs. 5M5NIId (both show E_{1/2} of −1.08 V vs. Fc⁺/Fc, Table 3). This observation is likely due to the charge-transfer effect that lowers the energy level of the molecule, offsetting the electronic effect from EDGs. More particularly, isoindigo species are electron acceptors (n-type organic semiconductors), Wei et al., 2021, where charge transfer can be induced between the electron-deficient isoindigo rings and the electron-rich methoxy group. UV-vis absorption spectra suggest an optical bandgap of 1.90-1.98 eV for most isoindigos with or without chemical modification (Table 3 and FIGS. 19-21). The bandgaps drop to 1.78 and 1.71 eV for methoxy substituted 55DMIId and 5M5NIId, however, manifesting charge transfer in these two compounds.

TABLE 1 [symbol]
The interplay between substituent groups and intramolecular hydrogen bonding in isoindigos and
the corresponding impacts on redox potentials (V vs. Fc⁺/Fc) and CO₂ binding.

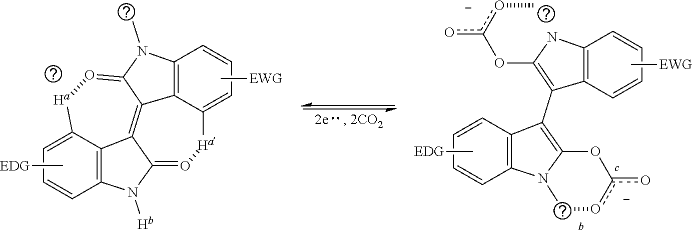

| | ¹H NMR (H [symbol] ) | ¹H NMR (H^{b}) | Bond length of a | Bond length of b | Bond length of c | E_{1/2} (IId/IId [symbol] ) in | |
|---|---|---|---|---|---|---|---|
| Isoindigos | (ppm)^{A} | (ppm)^{A} | (Å)^{B} | (Å)^{B} | (Å)^{B} | N₂ (V vs Fc [symbol] /Fc) | logK[CO₂] |
| IId | 9.06 | 10.89 | 1.963 | 1.954 | 1.461 | −1.29 | 9.34 |
| 55DMIId | 8.85 | 10.69 | 1.953 | 1.970 | 1.463 | −1.29 | 9.05 |
| 5BIId | 9.07 | 10.96 | 1.962 | 1.957 | 1.463 | −1.19 | 9.05 |
| 5BIId (EWG)^{c} | 9.31 | 11.05 | 1.936 | 1.943 | 1.467 | −1.19 | 9.05 |
| 55DBIId | 9.32 | 11.11 | 1.939 | 1.946 | 1.469 | −1.09 | 9.33 |
| 6MCIId | 9.07 | 10.94 | 1.960 | 1.954 | 1.464 | −1.12 | 8.89 |
| 6MCIId (EWG)^{c} | 9.15 | 11.08 | 1.956 | 1.944 | 1.476 | −1.12 | 8.89 |
| 66DBIId | 8.99 | 11.10 | 1.961 | 1.942 | 1.468 | −1.14 | 9.59 |
| NNDPr66DBIId | 9.05 | NA | 1.948 | NA | 1.486 | −1.07 | 4.87 |

[symbol]

H NMR were recorded on the neutral molecules in DMSO-d₆ using solvent residual peak as the internal reference for calibration. [symbol]
Bond length was obtained from DFT-optimized structures.
^{c}Results from the non-symmetric oxindole ring with the EWG-substituent. [symbol]
indicates data missing or illegible when filed

### 1.4.3 Breaking the Scaling Relationship Between Redox Potential and CO₂ Affinity

After understanding the effect of molecular structures on the redox potentials of isoindigos, we further studied their CO₂ binding behaviours. All isoindigos in this Example exhibited anodically shifted potential for the second electron transfer process under CO₂, confirming their ability to form CO₂ adducts upon electro-reduction. Importantly, through the combined effect of EWGs substitution and hydrogen bonding (a) discussed above, all isoindigos with unsubstituted Hb display E_{1/2} values anodic to oxygen reduction under CO₂, implying favourable stability of isoindigo sorbents against O₂.

In all previous reports on redox-tunable CO₂ sorbents, anodically shifted redox potential always comes with significantly diminished CO₂ binding affinity (Table 2). A key finding of this Example, however, is that hydrogen atom (Hb) on the lactam-N of isoindigos can induce intermolecular hydrogen bonding with the complexed CO₂ molecule to thermodynamically stabilise the CO₂ adduct (FIG. 4e). As a result, regardless of E_{1/2}, the log K_{CO}_{₂} values of isoindigos with unsubstituted Hb were found to be relatively constant (Table 1 and Table 3, see Section 1.9 for details on K_{CO}_{₂} calculation), suggesting the high tolerance of such redox carriers to a wide range of chemical modifications.

To further demonstrate the role of hydrogen bonding (b) on CO₂ adduct stabilisation, we synthesised two examples with N-substitutions to eliminate this hydrogen bonding (NNDPr66DBIId and NNDEHIId). Compared with 6,6′-dibromo substituted 66DBIId (log K_{CO}_{₂}=9.59), the CO₂ binding constant of N-alkylated NNDPr66DBIId (log K_{CO}_{₂}=4.48) shows a dramatic decrease of five orders of magnitude (Table 1). A similar phenomenon was observed between NNDEHIId (log K_{CO}_{₂}=6.62, Table 3) and IId (log K_{CO}_{₂}=9.34). Further, we designed an isoindigo bearing a carboxylic acid group (6CIId), which serves as a free proton donor to disrupt the intramolecular hydrogen bonding (b). As a result, the CO₂ binding ability of reduced 6CIId almost diminished with a low log K_{CO}_{₂} of 2.87 (Table 3).

To visualize the success in breaking the scaling relationship between redox potential and CO₂ binding affinity, the change in K_{CO}_{₂} relative to unmodified IId was plotted against E_{1/2} of the first electron transfer under N₂ (FIG. 5b). Upon installing EWGs, E_{1/2} can be effectively shifted from −1.29 V to −0.97 V vs. Fc⁺/Fc. Nevertheless, the K_{CO}_{₂}values exhibit minimal changes within only one order of magnitude, as long as the lactam-N is unsubstituted to facilitate intramolecular hydrogen bonding. Furthermore, linear fitting shows a negligible correlation (R₂<0.1), underlining that our molecular design strategy can indeed break the scaling relationship between redox potential and CO₂ binding affinity.

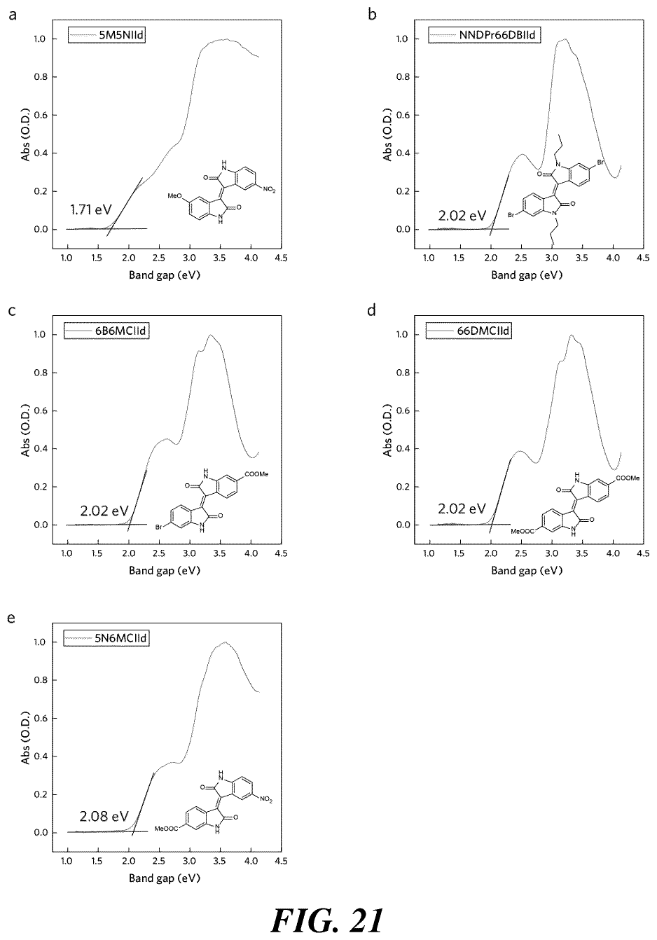

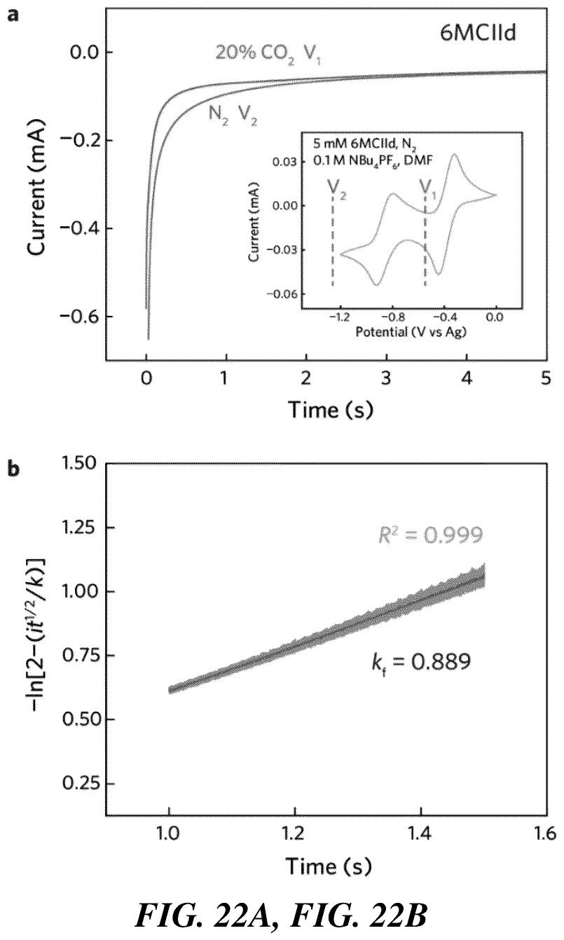

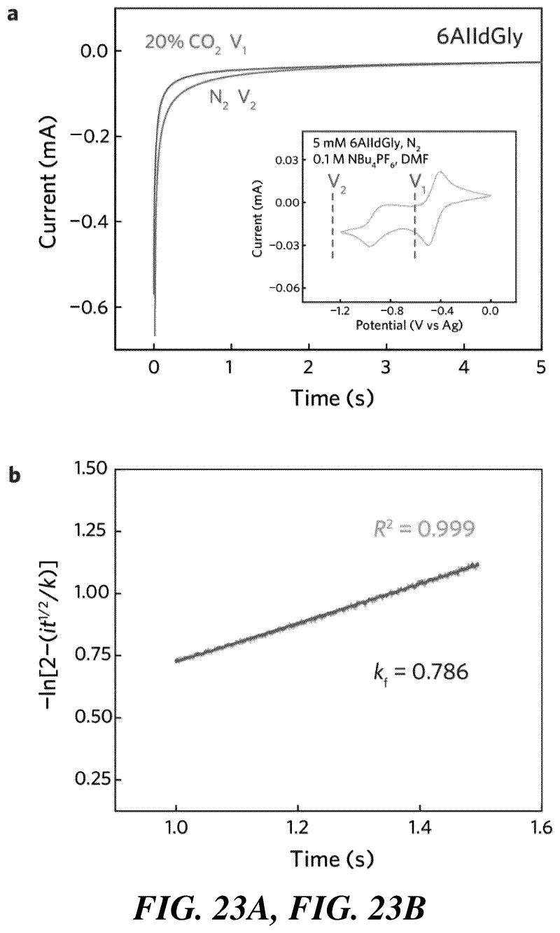

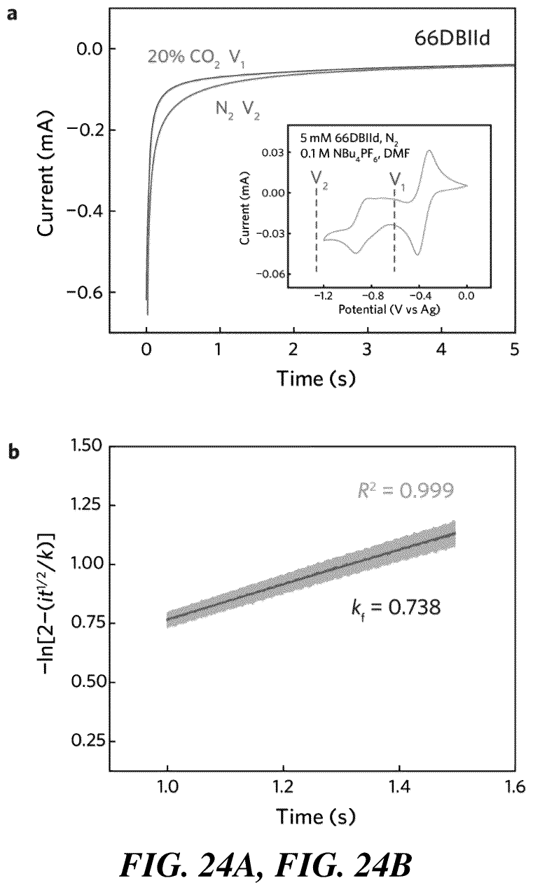

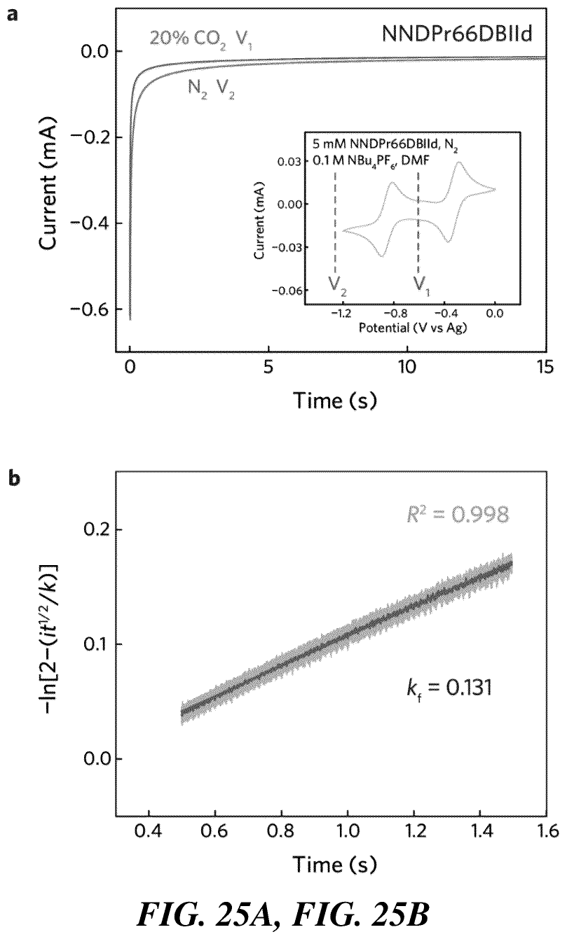

We further tested the bimolecular rate constant (k_{bimolecular}) for the reaction between isoindigo radical anion and CO₂ (FIGS. 22-25, Table 4, see Section 1.9 for details in measurement). With intramolecular hydrogen bonding (b) in the CO₂ adduct, 6MCIId, 6AIIdSer, and 66DBIId display a similar rate constant of 22.5-18.6 M⁻¹ s⁻¹. The N,N-disubstituted NNDPr66DBIId, however, exhibits a decreased rate constant of 3.3 M⁻¹ s⁻¹, manifesting that intramolecular hydrogen bonding (b) also is conducive to CO₂ complexation kinetics.

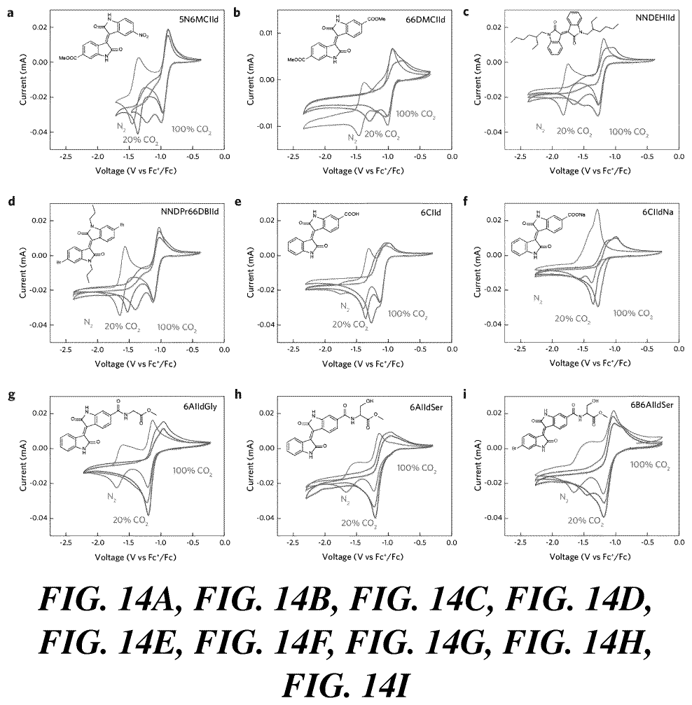

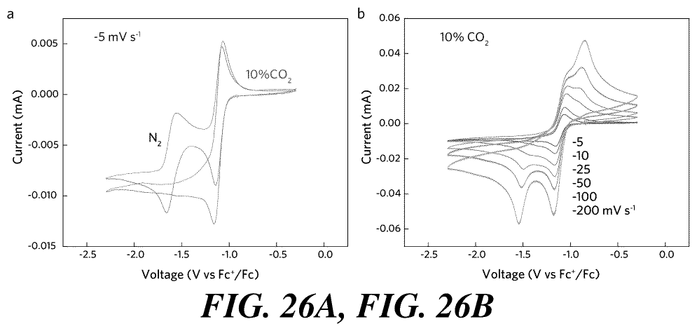

At relatively low CO₂ concentrations (20% or 10%), the CV curves of isoindigos usually exhibit a positively shifted second reduction peak compared to that under N₂; however, most do not completely emerge into the first (FIG. 13 and FIG. 14). The phenomenon is attributed to the kinetic competition between the chemical transformation of IId·−into [IId-CO₂]·− (r=k_{bimolecular}[CO₂][IId·−], Xu et al., 2023, where [CO₂] and [IId·−] are the concentrations of CO₂ and IId·−) and the electrochemical reduction of [IId-CO₂]·−. Higher CO₂ concentrations lead to higher r, which facilitates the formation of [IId-CO₂]·− and ultimately leads to a single, merged reduction peak. Correspondingly, we tested the CV of 6MCIId at different scan rates and indeed observed the gradual merging of the two reduction peaks with slower scans (FIG. 26). Therefore, the separation between the two cathodic peaks under low CO₂ concentrations can serve as a qualitative indicator for CO₂ complexation kinetics.

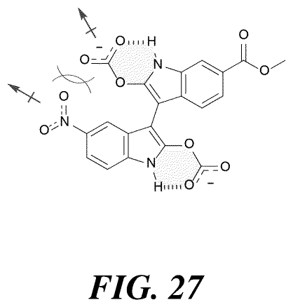

As a final note, CO₂ complexation can be frustrated when introducing extremely strong EWGs or strong hydrogen bonding acceptors to isoindigo (5N6MCIId and 6CIIdNa). Detailed analysis is included in Section 1.10 and FIG. 27.

### 1.4.4 Detailed Investigation on the Role of Hydrogen Bonding

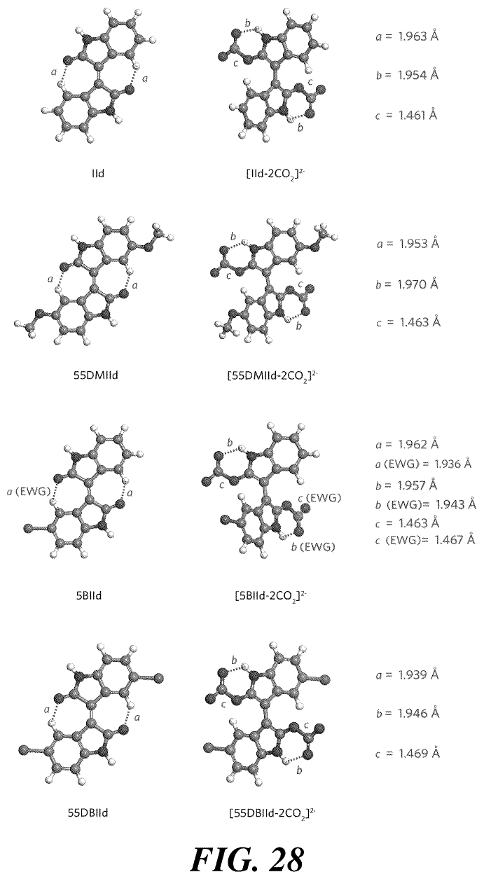

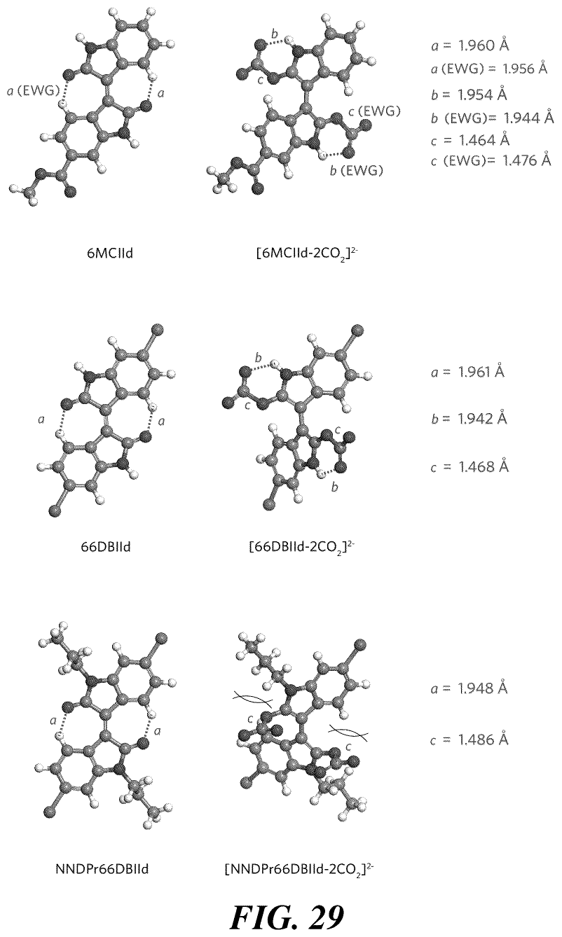

Distinct from previously reported redox-tunable CO₂ carriers, isoindigos possess intramolecular hydrogen bonding in both inactivated (neutral) and activated (reduced) forms, accounting for their unique EMCC properties. Therefore, we selected the seven most representative structures from our library and carefully compared various parameters, such as NMR spectra, density functional theory (DFT)-optimised bond lengths, redox potentials, and CO₂ binding constants, to investigate the interplay between substituent groups and hydrogen bonding on the thermodynamic properties of isoindigos. Key data are summarised in Table 1 (also see FIG. 28 and FIG. 29 for DFT-optimised structures). DFT calculation shows that the theoretical first electron transfer potential shifts anodically in the order of IId, 5BIId, 66DBIId, and 55DBIId, consistent with experimental observation (Table 5). Further, DFT-calculated CO₂ binding energies for the two-electron reduced isoindigos agree well with ΔEpeak(2) calculated from CV experiments (Table 6). DFT-optimised structures further confirm the formation of intramolecular hydrogen bonding (a) and (b).

The electron density of Ha at 4-position and Hb at N-position can be regarded as indicators of the bond strength of a and b, respectively. Specifically, 1H NMR spectra reveal a chemical shift of 9.06 ppm for Ha and 10.89 ppm for Hb in unmodified IId, corresponding to a DFT-optimised bond length of 1.963 Å for a and 1.954 Å for b. Introducing electron-withdrawing bromo groups at 5-position (55DBIId) downfield shifts Ha to 9.32 ppm and Hb to 11.11 ppm, resulting in an enhanced hydrogen bonding of 1.939 Å for a and 1.946 Å for b, respectively. Interestingly, the electronics and hydrogen bonding of each oxindole ring can be independently tuned in nonsymmetric isoindigos. Using the nonsymmetric 5BIId as an example, the chemical shifts of Ha and Hb are 9.07 and 10.96 ppm at the non-substituted side, corresponding to a DFT-optimised bond length of 1.962 Å for a and 1.957 Å for b, which is very close to IId. In contrast, Ha and Hb shift downfield to 9.31 and 11.05 ppm at the bromo-substituted side, corresponding to an enhanced hydrogen bonding of 1.936 Å for a and 1.943 Å for b.

Moreover, introducing the same EWG at different positions modulates the strength of hydrogen bonding (a) differently. For example, with the same dibromo-substitution, the chemical shift of Ha in 66DBIId (8.99 ppm) is upfield to that of 55DBIId (9.32 ppm). This substitution results in a weakened hydrogen bonding (a) of 1.961 Å in 66DBIId than that of 1.939 Å in 55DBIId, explaining the more negative reduction potential of 66DBIId (−1.14 V vs. Fc⁺/Fc) than 55DBIId (−1.09 V vs. Fc⁺/Fc). The above results strongly suggest that, in addition to the electronic effect of substituent groups, intramolecular hydrogen bonding (a) also is vital in facilitating the reduction of isoindigo.

Although decreasing the electron density of isoindigos by introducing EWGs at 5,6-positions can effectively facilitate their reduction, counterintuitively, the reduced isoindigos exhibit negligible decay in CO₂ affinities, underscoring the importance of intramolecular hydrogen bonding (b). Using DFT calculation, we found that the nucleophilicity of the oxygen centre indeed weakens when EWG is introduced, as suggested by the increased length of the carbonate C—O bond (c). For instance, the bond length of c on the oxindole ring with —COOMe group is increased by 1.2 pm compared to that on the non-substituted ring in 6MCIId. As mentioned above, however, hydrogen bonding (b) strengthens with stronger or increasing number of EWG substituents to keep the bond length of c nearly constant. Moreover, the DFT-optimised structure suggests that hydrogen bonding (b) is precluded in NNDPr66DBIId, and the carbonate bends out-of-plane to the reduced isoindigo rings due to steric repulsion. Thus, the bond length of c in NNDPr66DBIId is increased by 1.8 pm compared to 66DBIId, giving rise to a significant drop in K_{CO}_{₂} by almost five orders of magnitude.

The collective information above confirms conclusions from the Example. First, hydrogen bonding (a) can reduce the electron density at the redox centre and facilitate reduction. Second, hydrogen bonding (a) can be tuned by substituent groups, where EWG at 5-position is more effective than 6-position to shorten the bond length of a and hence facilitate reduction. Third, hydrogen bonding (b) stabilises the complexed CO₂ when EWGs are introduced.

It is important to note that the effect of intramolecular hydrogen bonding on CO₂ complexation has been briefly studied in prior works using quinones. It was observed, however, that hydrogen bonding occupies the CO₂ binding sites of quinones and diminishes the ability for CO₂ capture. Nagaoka et al., 1992; Schimanofsky et al., 2022.

Therefore, this Example presents the first demonstration that intramolecular hydrogen bonding can facilitate CO₂ adduct formation and break the intrinsic linear free-energy relationship of EMCC chemistries. This characteristic is attributed to the unique chemical structure of isoindigo that allows free rotation of the oxindole rings in the reduced state as supported by DFT simulation, breaking the intramolecular hydrogen bonding (a) to create space for CO₂ complexation, which is further enhanced by the intramolecular hydrogen bonding (b) through the amide functionality.

### 1.4.5 Finetuning the Properties of Isoindigos

Breaking the correlation between chemical modification and CO₂ binding affinity greatly enhances the degree of freedom in finetuning sorbent properties. For instance, by installing EWGs, such as methyl carboxylate, E_{1/2} of 66DMCIId under CO₂ can be positively shifted by 300 mV compared to that of unmodified IId to impart 02 stability, while the log K_{CO}_{₂} only drops slightly (from 9.34 to 8.07). Further, 66DMCIId is almost insoluble in organic solvents such as DMF (solubility <2.5 mM), suggesting its potential as absorbent electrodes in fixed-bed EMCC devices.

Unmodified IId has a moderate solubility in DMF (approximately 230 mM), which needs to be improved for practical use in flow-based EMCC systems. Liu et al., 2022. To facilitate chemical functionalisation, we introduced a carboxyl group to the 6-position of isoindigos, which can be easily connected with amino acids through amidation reactions. As a proof of concept, we utilised glycine methyl ester and serine methyl ester as solubility enhancers and three nonsymmetric isoindigos were prepared (6AIIdGly, 6AIIdSer, and 6B6AIIdSer). 6B6AIIdSer features halogen substitution on one oxindole ring to tune redox potentials and amino acid ester functionalisation on the other oxindole ring to enhance solubility. The solubilities of 6AIIdGly, 6AIIdSer, and 6B6AIIdSer increase to 606, 830, and 568 mM in DMF, respectively (Table 7), likely due to the enhanced molecular interaction between the polar functional groups (amide and carboxylate) and DMF solvent. This hypothesis is supported by the fact that 6AIIdSer is more soluble than 6AIIdGly due to the additional hydroxyl group from the serine moiety.

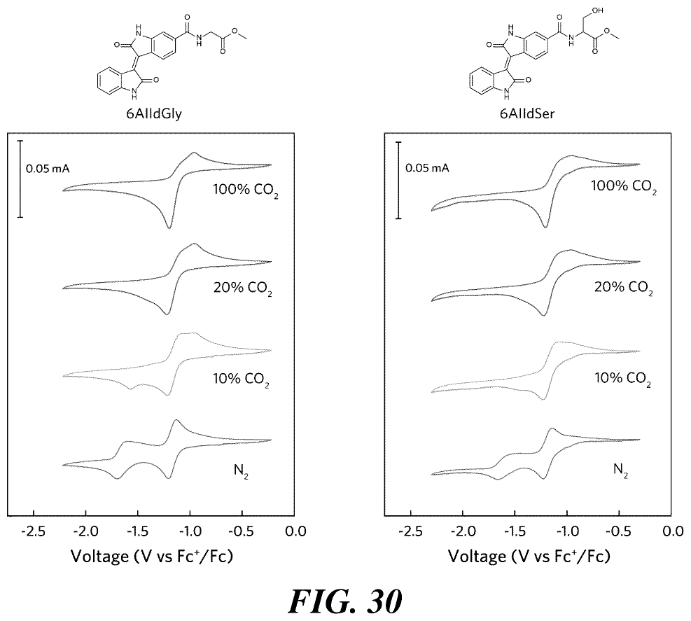

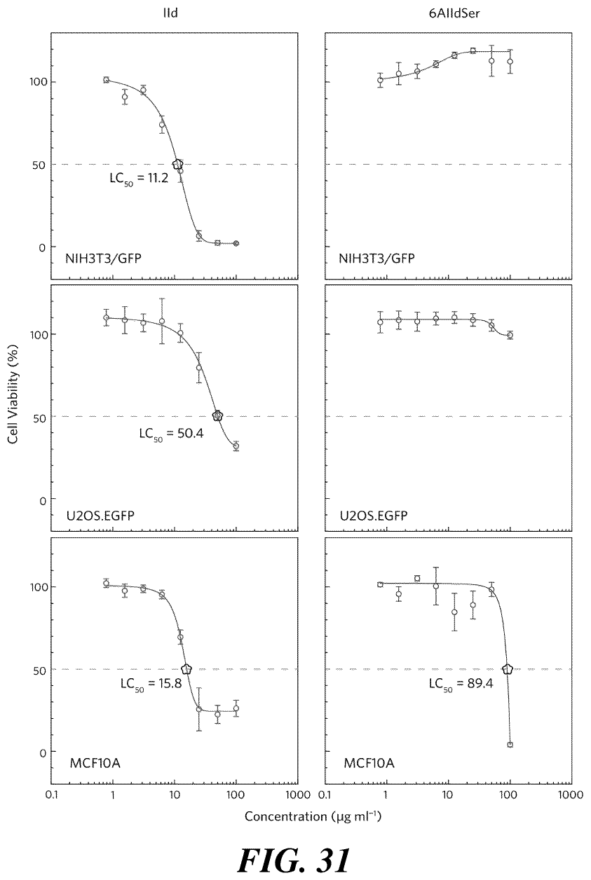

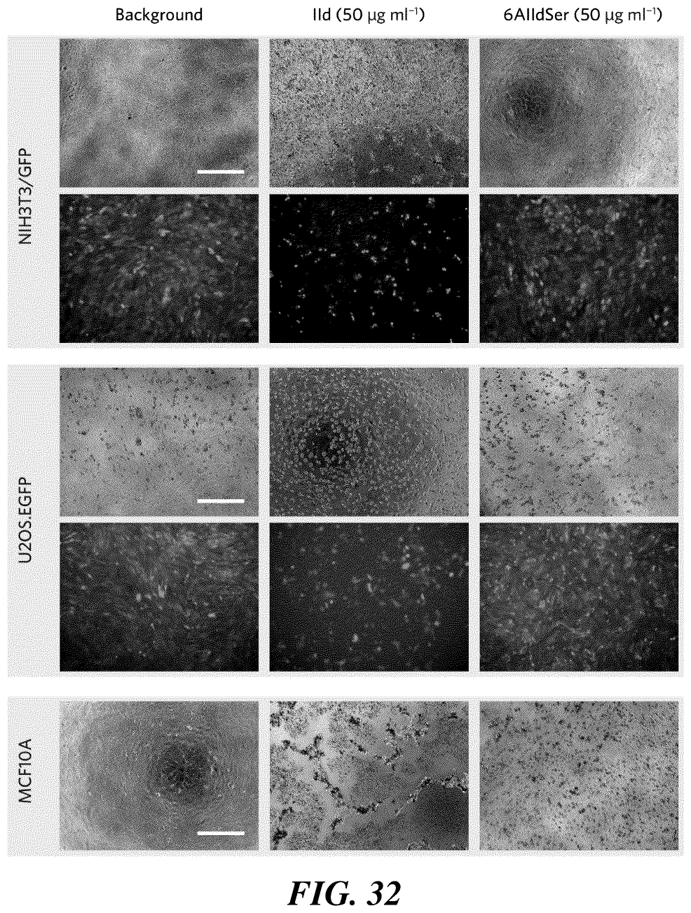

Conventional amine scrubbing sorbents have raised environmental concerns due to their biotoxicity. del Rio et al., 2019; Rohr et al., 2013. This Example demonstrates that introducing amino ester functionalities into isoindigos substantially improves their biocompatibility with mammalian cells. Unmodified IId shows a LC₅₀ (lethal concentration that causes 50% cell death) of 11.2, 50.4, and 15.8 g mL⁻¹ for NIH3T3/GFP mouse fibroblasts, U2OS.EGFP human osteosarcoma cells, and MCF10A human breast epithelial cells, respectively, after 48 h cell culture (FIG. 30 and FIG. 31). In comparison, the serinate-modified counterpart does not display clear toxicity under concentrations up to 100 g mL⁻¹ for NIH3T3/GFP and U2OS.EGFP, and has a significantly improved LC₅₀ of 89.4 g mL⁻¹ for MCF10A.

### 1.4.6 Effects of Electrolytes, Oxygen, and Water

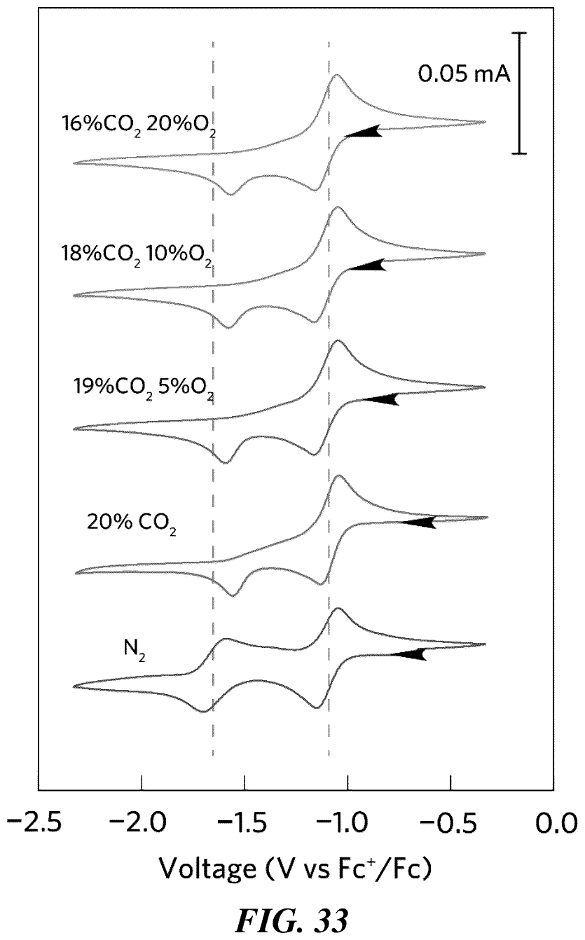

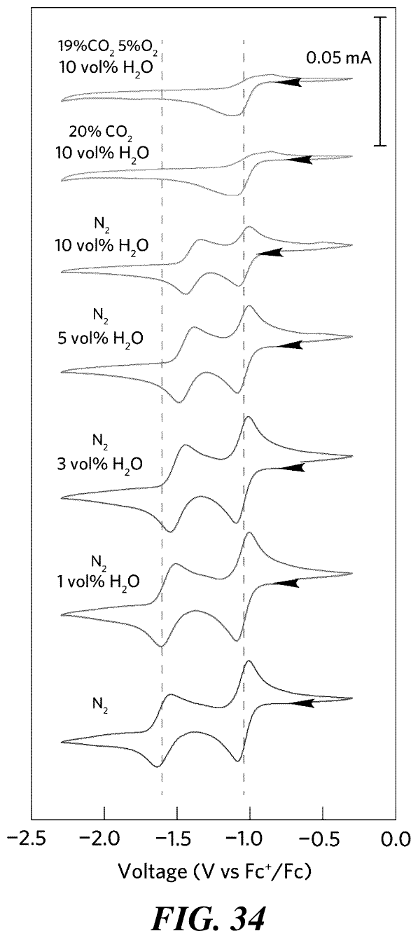

Before evaluating the CO₂ capture performance of isoindigos in EMCC devices, we assessed the influence of electrolytes and common gas stream impurities, such as water and O₂, on their CO₂ binding properties. Using 55DBIId as an example, the electrochemical behaviours remained almost unaffected up to a high O₂ content (16% CO₂ and 20% O₂, FIG. 32). Under N₂, the CV curves of 55DBIId remain reversible even at a high water content of 10 vol % (FIG. 33). The declining peak current is caused by the decreasing isoindigo solubility with increasing water content. The expected CO₂ release at approximately −1 V vs. Fc⁺/Fc, however, gets suppressed in the presence of 10 vol % water, possibly due to the involvement of pH-swing process under high water content that requires higher energy input for CO₂ release.

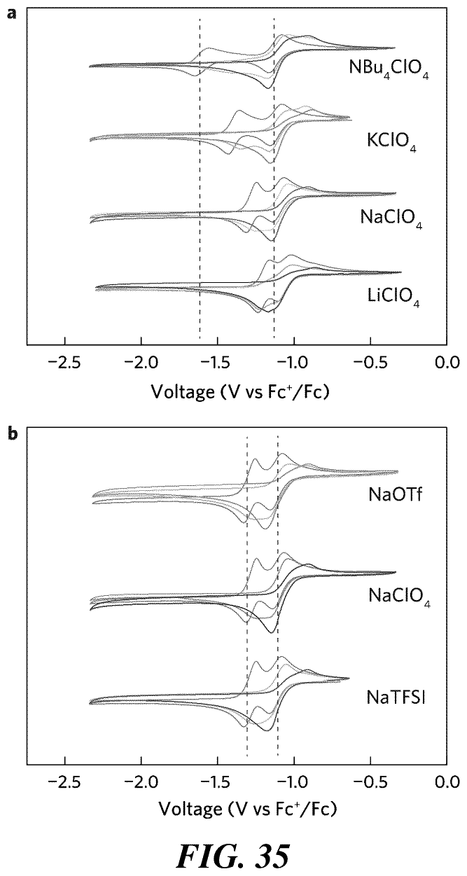

We also studied the influence of supporting salt on the redox and CO₂ binding behaviours of isoindigos (FIG. 35). The most prominent effect comes from the choice of cation, where reducing the size of cation leads to anodically shifted reduction potential, as is explained by the electrostatic interaction between cation and reduced isoindigo. Smaller alkaline cations exhibit stronger Lewis acidity, allowing tighter binding with reduced isoindigo to facilitate electro-reduction. Therefore, a more acidic supporting salt cation can further enhance the robustness of isoindigo against O₂. Nevertheless, it might also slow down the CO₂ complexation kinetics due to the competition between cation and CO₂ for binding with reduced isoindigo.

### 1.4.7 Evaluating the Isoindigo Sorbents in Flow-Based EMCC Prototypes

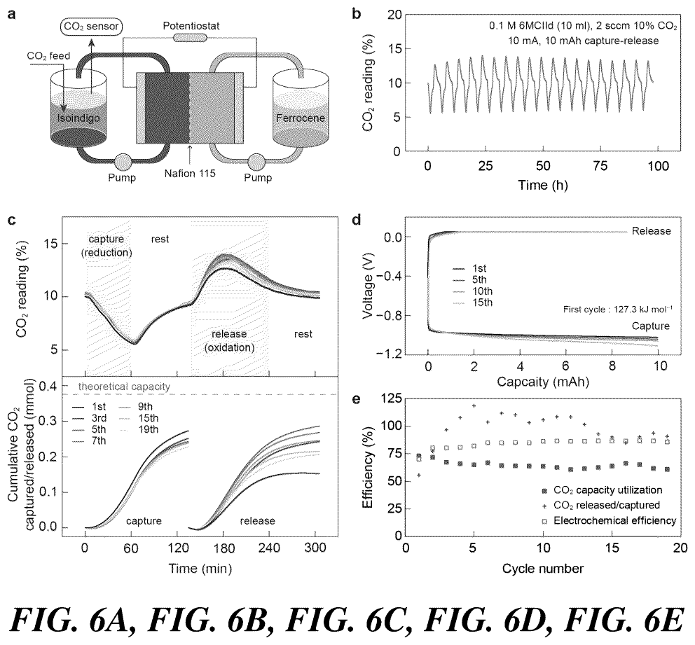

Based on the CV peak potentials for CO₂ capture and release, we estimated the theoretical minimum energy requirement for CO₂ separation using isoindigo sorbents, which ranges from 9.8 to 27.6 kJ mol⁻¹ CO₂ (FIG. 35). It is noteworthy that these molecules show very close onset potentials for CO₂ capture and release such that the theoretical energetics calculated from CV peak potentials can be overestimated compared to previous calculations using onset values. Gurkan et al., 2015. As a proof of concept, we evaluated their intrinsic capability for reversible CO₂ capture and release in a flow-based EMCC prototype reported by us previously (FIG. 6a). Gurkan et al., 2015.

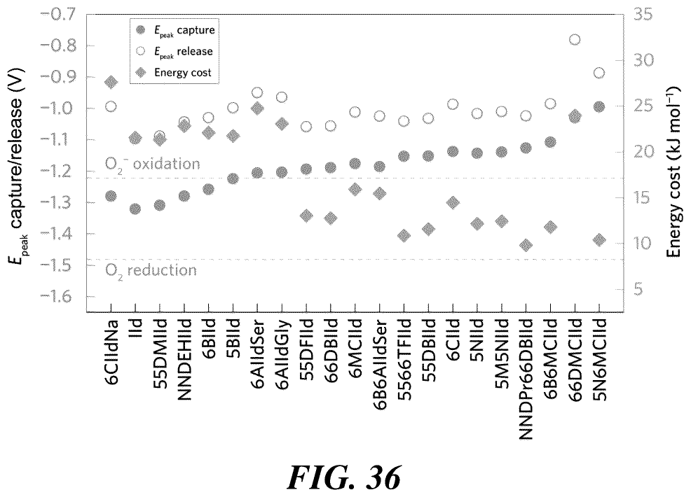

FIG. 6b shows the cyclic capture-release performance of 6MCIId using 10% CO₂ (balanced by N₂) as the gas feed. The CO₂ reading curves of each cycle are overlaid in FIG. 6c, where the cumulative CO₂ captured/released is obtained by integrating these curves. For each cycle, 6MCIId was reduced at 10 mA for 60 minutes, and the decrease in CO₂ concentration at the gas outlet confirmed carbon capture. The current was then stopped for 75 minutes, allowing the CO₂ reading to gradually return to the baseline as the reduced isoindigo fully reacted with CO₂. The CO₂ adduct was subsequently oxidised following a constant current-constant voltage (CC-CV) protocol, and the continuous increase in CO₂ concentration above 10% indicated CO₂ desorption. Afterward, the current was set to zero again to ensure the complete release of the oversaturated CO₂ from the sorbent electrolyte. The oxidation-reduction profiles of the flow system are shown in FIG. 6d. By integrating the voltage-capacity curves, the electrical energy consumption under 10% CO₂ is estimated as 127.3 kJ mol⁻¹ CO₂ in the first cycle and 142.5±8.2 kJ mol⁻¹ CO₂ over the first 16 cycles (FIG. 36), which is comparable to other carbon capture technologies. Voskian and Hatton, 2019; Diederichsen et al., 2021; Li et al., 2022; Eisaman et al., 2011; Wang et al., 2019; Wang et al., 2020; Jin et al., 2020; Jin et al., 2022; Liu et al., 2020.

FIG. 6e summarises the three key metrics commonly used for evaluating EMCC performance. CO₂ capacity utilisation, defined as the amount of CO₂ captured relative to the theoretical value (one CO₂ per electron), shows an average of 65% over 19 cycles, which is competitive against the state-of-the-art quinone-based sorbent reported recently. Diederichsen et al., 2021.

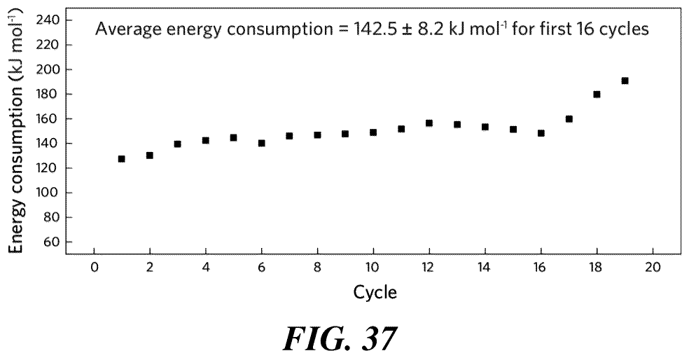

The release/capture efficiency, defined as the ratio of the total amount of CO₂ released and captured in each cycle, is averaged to be 97%, indicating the good reversibility of the sorbent. Considering a relatively constant electrochemical (Coulombic) efficiency of the EMCC prototype at approximately 84%, the major loss should be attributed to two factors: (1) our CC-CV protocol where the CO₂ adduct was not fully oxidised; (2) the crossover of sorbents and counter electrolytes caused by membrane swelling, which limits all current nonaqueous redox-flow electrochemical systems. 6MCIId also was evaluated at a higher percentage of CO₂ removal (FIG. 37).

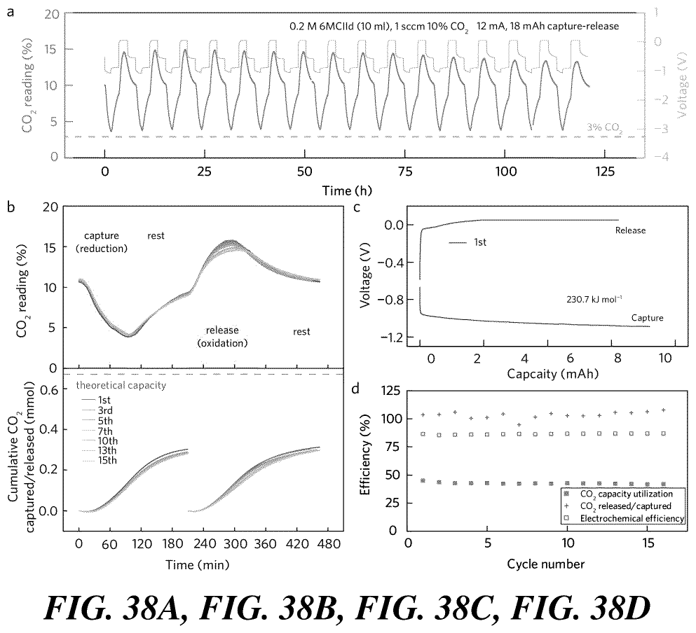

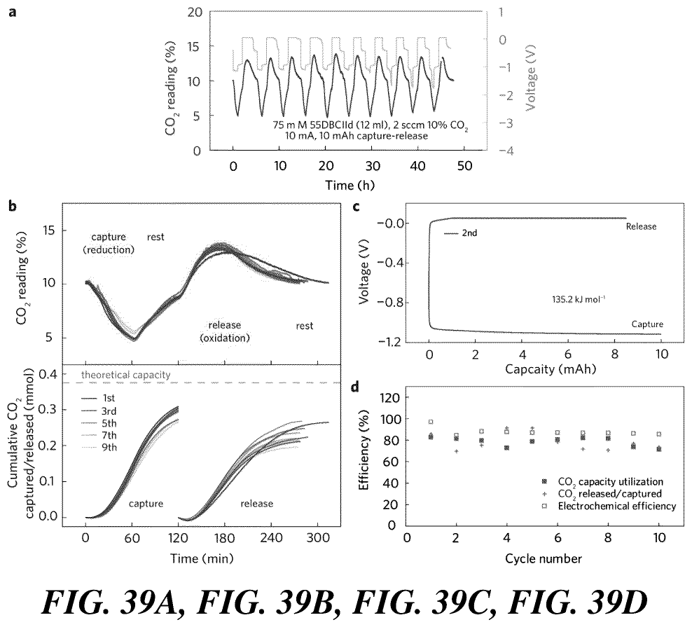

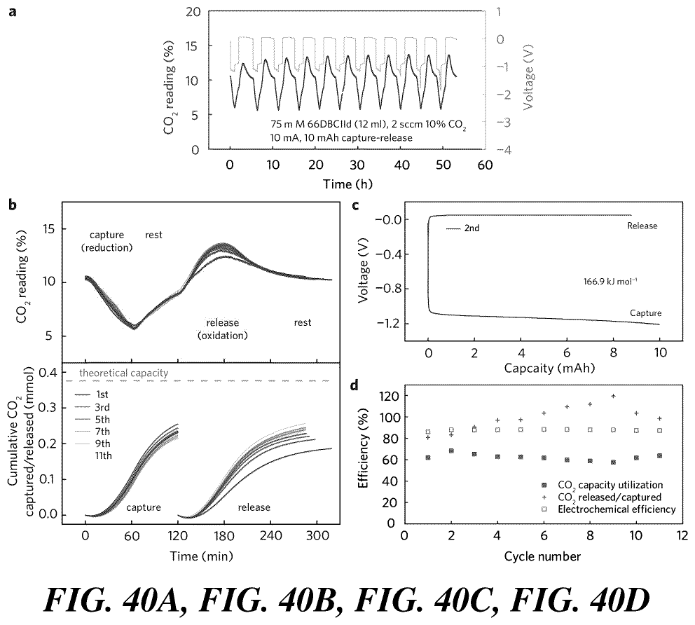

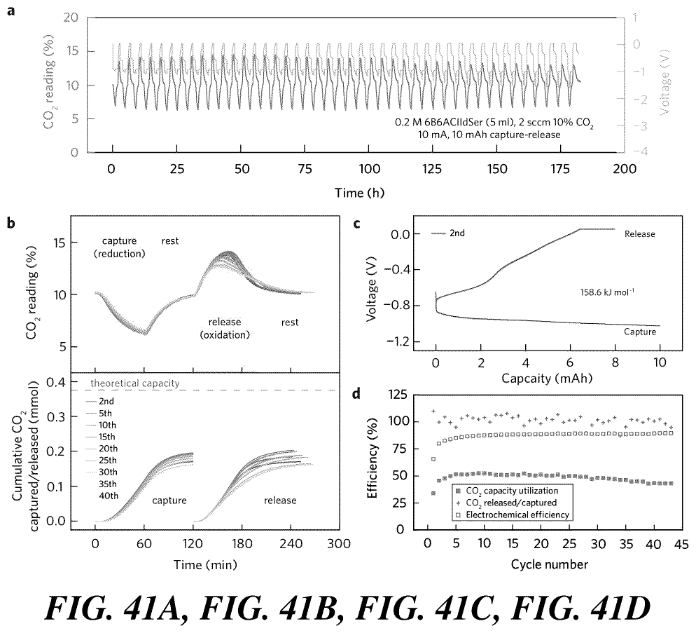

In addition to 6MCIId, we evaluated the performance of other isoindigo sorbents, such as 55DBIId (FIG. 38), 66DBIId (FIG. 39), and 6B6AIIdSer (FIG. 46). 55DBIId achieved an average CO₂ utilisation efficiency of up to approximately 80% and an average release/capture efficiency of approximately 80%. Using 66DBIId, we studied ¹H NMR of the crude sorbent electrolyte after 11 capture/release cycles over 50+ hours (FIG. 41). The spectrum suggests the high stability of 66DBIId after cycling and also the severe crossover issue of the ferrocene counter electrolyte (three times the concentration of 66DBIId in the sorbent tank), which explains the decay in electrochemical capacity of current EMCC prototypes. Further, we can recover 66DBIId with 87% yield from the sorbent electrolyte after cycling, corroborating the robustness of the sorbent. In addition, we found approximately 24% of 66DBIId isomerized into cis-66DBIId, supporting our hypothesis on the rotational isomerization of reduced 66DBIId discussed earlier (FIG. 16). Nevertheless, the reduction of both 66DBIId and its cis-isomer yield the same activated CO₂ sorbent, which we believe does not affect the long-term stability of the EMCC prototype.

6B6AIIdSer exhibits a much lower oxidation potential for CO₂ release, likely due to its higher solubility. Moreover, 6B6AIIdSer shows excellent cycling stability with negligible voltage decay over more than 40 cycles and approximately 200 hours of operation with a degradation rate of 2%.

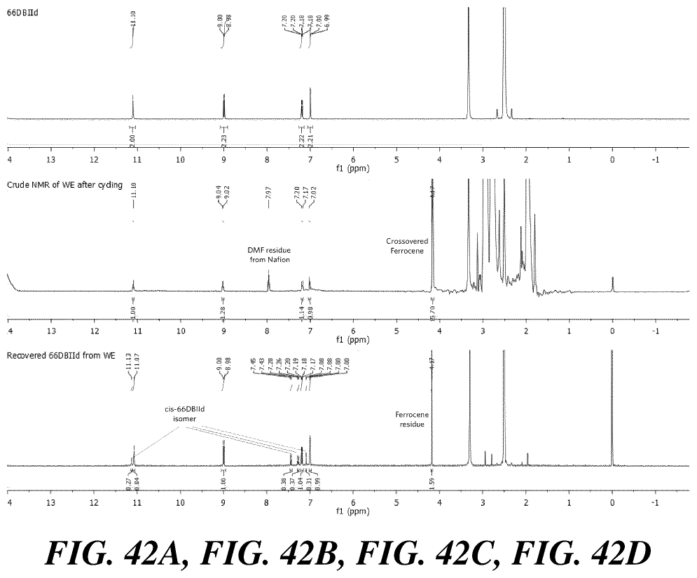

We further tested the EMCC performance of 6MCIId using simulated flue gas (10% CO₂+3% O₂ balanced in N₂) (FIG. 42). The cell can run stably over approximately 90 hours with reduction voltage maintained above −1.3 V, which minimised the parasitic oxygen reduction reaction. A CO₂ capacity utilisation efficiency of approximately 50% was achieved with a near unity CO₂ release/capture efficiency.

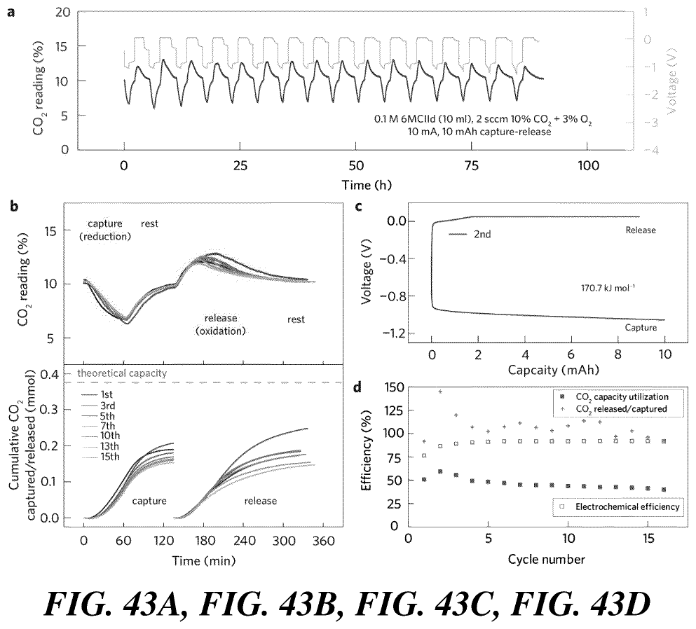

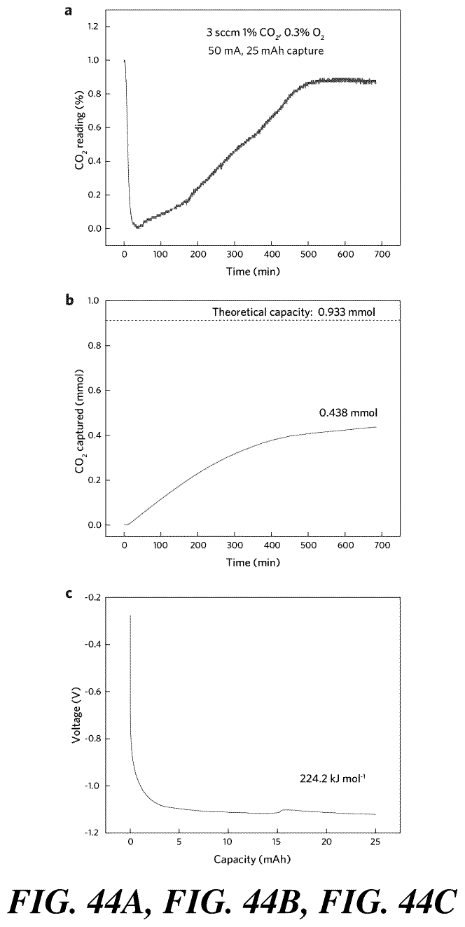

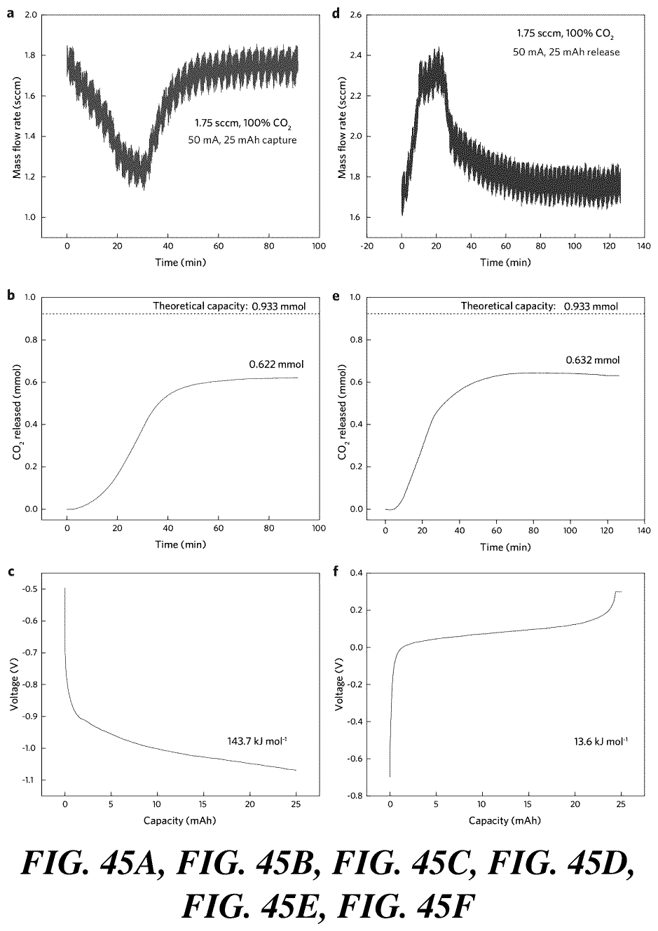

As a final note, we evaluated the CO₂ capture capability of 6MCIId under low CO₂ concentration and its CO₂ release capability under pure CO₂. Using 1% CO₂ with 0.3% O₂ as the feed, we observe an early-stage energy consumption of 224.2 kJ mol⁻¹ CO₂ and a single pass CO₂ removal of greater than 90% (FIG. 43). This observation suggests that our intramolecular hydrogen bonding strategy is effective in improving CO₂ affinity for low-concentration CO₂ capture. In another experiment, the CO₂ capture-release behaviour under 100% CO₂ headspace was quantified using mass flow metre (FIG. 44). Under conditions similar to low-concentration CO₂ capture, we show an early-stage energy consumption of 143.7 kJ mol⁻¹ CO₂ captured and 13.6 kJ mol⁻¹ CO₂ released, respectively.

In this Example, we focused on exploring the fundamental chemistry of isoindigos as redox-active CO₂ carriers and their potential to overcome the linear free-energy relationship that limits the structural modification of EMCC sorbents. The flow-based prototype in this Example, however, is not an ultimate design for practical systems but a proof-of-concept demonstration to evaluate the performance of isoindigo at the lab scale. We believe future efforts can substantially improve the performance by optimising the electrolytes, electrodes, and membranes of EMCC devices.

### 1.5 Discussion

This Example demonstrates the rational design of a new class of bifunctional redox-tunable CO₂ carriers based on isoindigo and derivatives thereof. The unique intramolecular hydrogen bonding in isoindigo moieties enables a wide range of chemical modifications to facilitate electro-reduction, tune solubility, and preclude parasitic reactions without compromising their CO₂ binding ability. With coupled experimental and computational studies, we provide an in-depth analysis of the structure-function relationships of isoindigos as EMCC sorbents. Compared to existing EMCC sorbents, the presently disclosed isoindigo compounds have the following advantages: 1) a nearly constant log K_{CO}_{₂} of approximately 9 with high tolerance to chemical modifications; 2) facile synthesis with the ease of encoding functionalities; 3) highly tunable redox potentials and solubilities; 4) good biocompatibility.

In addition to flow-based EMCC, we envisage that isoindigos also can find applications as solid adsorbents in fixed-bed systems, due to their descent charge mobility and the abundant methods in synthesising and processing isoindigo-based polymers developed by the community of organic semiconductors. The presently disclosed subject matter paves the way for engineering more reliable EMCC systems by breaking the fundamental barriers of the scaling relationship between redox potential and CO₂ binding strength when designing redox-tunable CO₂ sorbents.

### 1.6 Methods

### 1.6.1 Nuclear Magnetic Resonance Spectroscopy (NMR)

NMR spectra were collected on a Bruker AMX400 (400 MHz) spectrometer. Chemical shifts were reported in parts per million (ppm). Residual solvent peak was used as an internal reference.

### 1.6.2 Ultraviolet-Visible (UV-Vis) Spectroscopy

UV-vis absorption spectra were recorded on a Thermo Scientific Genesys 10S UV-vis spectrophotometer.

### 1.6.3 Synthesis of Isoindigos

### 1.6.3.1 General Synthetic Procedure for Isoindigos:

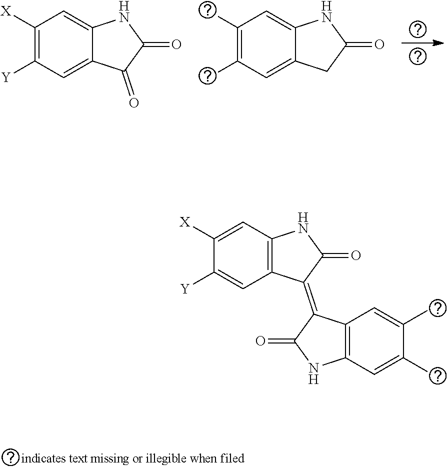

To a mixture of isatin (1.106˜20 mmol, 1.0 equiv) and 2-oxindole (1.106˜20 mmol, 1.0 equiv) in acetic acid (˜87 equiv) was added 37% HCl solution (1 vol % of acetic acid). The suspension was heated at reflux for 1 to 3 days under Ar atmosphere before being cooled to room temperature (most reactions were completed in 1 day except for those substrates with strong electron-withdrawing groups which required further reaction time). The mixture was filtered and washed with copious water, ethanol, and ethyl acetate (EA). The product was dried in the vacuum oven at 60° C. for 15 h to afford the desired isoindigos in 50˜97% yield.

### 5,5′-Dimethoxylisoindigo (55DMIId)

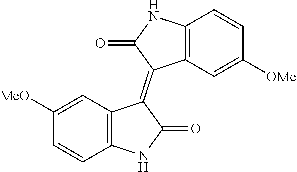 [SMILES: COc1ccc2c(c1)/C(=C1\C(=O)Nc3ccc(OC)cc31)C(=O)N2]

The target was synthesised by the general procedure starting with 5-methoxyisatin (209 mg, 1.18 mmol) and 5-methoxy-2-oxindole (192.6 mg, 1.18 mmol) in a day to afford the desired product (324 mg, 85%). ¹H NMR (400 MHz, DMSO) δ 10.69 (s, 2H), 8.85 (d, J=2.6 Hz, 2H), 6.97 (dd, J=8.5, 2.7 Hz, 2H), 6.75 (d, J=8.5 Hz, 2H), 3.73 (s, 6H).

### Isoindigo (IId)

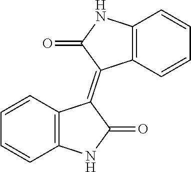 [SMILES: O=C1Nc2ccccc2/C1=C1\C(=O)Nc2ccccc21]

The target was synthesised by the general procedure starting with isatin (2.94 g, 20 mmol) and 2-oxindole (2.66 g, 20 mmol) in a day to afford the desired product (5.01 g, 96%). ¹H NMR (400 MHz, DMSO) δ 10.89 (s, 2H), 9.06 (d, J=7.9 Hz, 2H), 7.34 (t, J=7.6 Hz, 2H), 6.96 (t, J=7.8 Hz, 2H), 6.84 (d, J=7.7 Hz, 2H). ¹³C NMR (101 MHz, DMSO) δ 168.92, 144.03, 133.27, 132.54, 129.25, 121.65, 121.07, 109.46.

### 5,5′-Difluoroisoindigo (55DFIId)

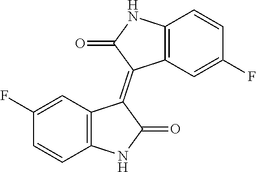 [SMILES: O=C1Nc2ccc(F)cc2/C1=C1\C(=O)Nc2ccc(F)cc21]

The target was synthesised by the general procedure starting with 5-fluoroisatin (195 mg, 1.18 mmol) and 5-fluoro-2-oxindole (178.3 mg, 1.18 mmol) in a day to afford the desired product (177 mg, 50%). ¹H NMR (400 MHz, DMSO) δ 11.02 (s, 2H), 9.00 (dd, J=11.4, 2.7 Hz, 2H), 7.25 (td, J=8.7, 2.8 Hz, 2H), 6.85 (dd, J=8.6, 4.9 Hz, 2H).

### 5-Bromoisoindigo (5BIId)

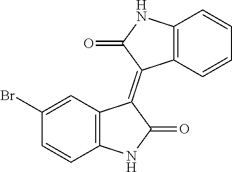 [SMILES: O=C1Nc2ccccc2/C1=C1\C(=O)Nc2ccc(Br)cc21]

The target was synthesised by the general procedure starting with isatin (323.7 mg, 2.2 mmol) and 5-bromo-2-oxindole (466.5 mg, 2.2 mmol) in a day to afford the desired product (691 mg, 92%). ¹H NMR (400 MHz, DMSO) δ 11.05 (s, 1H), 10.96 (s, 1H), 9.31 (d, J=1.8 Hz, 1H), 9.07 (d, J=8.0 Hz, 1H), 7.52 (dd, J=8.3, 2.0 Hz, 1H), 7.37 (t, J=7.6 Hz, 1H), 6.98 (t, J=7.8 Hz, 1H), 6.83 (dd, J=16.1, 8.0 Hz, 2H).

### 6-Bromoisoindigo (6BIId)

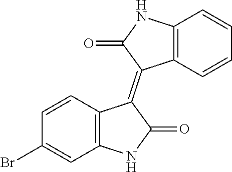 [SMILES: O=C1Nc2ccccc2/C1=C1\C(=O)Nc2cc(Br)ccc21]

The target was synthesised by the general procedure starting with 6-bromoisatin (250 mg, 1.106 mmol) and 2-oxindole (147 mg, 1.106 mmol) in a day to afford the desired product (261 mg, 69%). ¹H NMR (400 MHz, DMSO) δ 11.06 (s, 1H), 10.95 (s, 1H), 9.04 (d, J=7.2 Hz, 1H), 9.00 (d, J=8.9 Hz, 1H), 7.36 (s, 1H), 7.18 (d, J=7.1 Hz, 1H), 6.98 (d, J=8.6 Hz, 2H), 6.84 (d, J=7.5 Hz, 1H).

### 6,6′-Dibromoisoindigo (66DBIId)

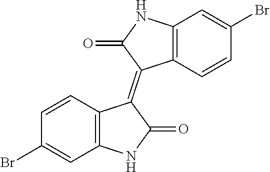 [SMILES: O=C1Nc2cc(Br)ccc2/C1=C1\C(=O)Nc2cc(Br)ccc21]

The target was synthesised by the general procedure starting with 6-bromoisatin (533 mg, 2.36 mmol) and 6-bromo-2-oxindole (500 mg, 2.36 mmol) in a day to afford the desired product (776 mg, 78%). ¹H NMR (400 MHz, DMSO) δ 11.10 (s, 2H), 8.99 (d, J=8.7 Hz, 2H), 7.18 (dd, J=8.7, 2.0 Hz, 2H), 6.99 (d, J=2.0 Hz, 2H).

### 5,5′-Dibromoisoindigo (55DBIId)

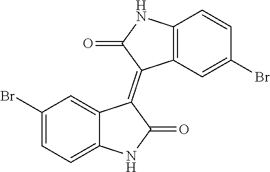 [SMILES: O=C1Nc2ccc(Br)cc2/C1=C1\C(=O)Nc2ccc(Br)cc21]

The target was synthesised by the general procedure starting with 5-bromoisatin (2.132 g, 9.44 mmol) and 5-bromo-2-oxindole (2 g, 9.44 mmol) in a day to afford the desired product (3.87, 97%). ¹H NMR (400 MHz, DMSO) δ 11.11 (s, 2H), 9.32 (d, J=2.0 Hz, 2H), 7.55 (dd, J=8.3, 2.1 Hz, 2H), 6.83 (d, J=8.3 Hz, 2H).

### 5-Nitroisoindigo (SNIId)

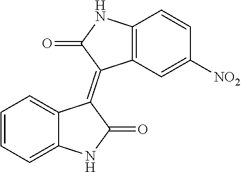 [SMILES: O=C1Nc2ccccc2/C1=C1\C(=O)Nc2ccc([N+](=O)[O-])cc21]

The target was synthesised by the general procedure starting with 5-nitroisatin (227 mg, 1.18 mmol) and 2-oxindole (157.1 mg, 1.18 mmol) in three days to afford the desired product (339 mg, 93%). ¹H NMR (400 MHz, DMSO) δ 11.63 (s, 1H), 11.03 (s, 1H), 10.12 (d, J=2.3 Hz, 1H), 9.08 (d, J=8.1 Hz, 1H), 8.29 (dd, J=8.7, 2.4 Hz, 1H), 7.40 (t, J=7.6 Hz, 1H), 7.02 (dd, J=15.7, 8.0 Hz, 2H), 6.88 (d, J=7.6 Hz, 1H).

### 5-Methoxyl-5′-nitroisoindigo (5M5NIId)

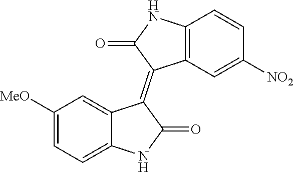 [SMILES: COc1ccc2c(c1)/C(=C1\C(=O)Nc3ccc([N+](=O)[O-])cc31)C(=O)N2]

The target was synthesised by the general procedure starting with 5-nitroisatin (227 mg, 1.18 mmol) and 5-methoxy-2-oxindole (192.6 mg, 1.18 mmol) in three days to afford the desired product (309 mg, 78%). ¹H NMR (400 MHz, DMSO) δ 11.61 (s, 1H), 10.84 (s, 1H), 10.14 (d, J=2.3 Hz, 1H), 8.83 (d, J=2.6 Hz, 1H), 8.29 (dd, J=8.7, 2.4 Hz, 1H), 7.08-6.98 (m, 2H), 6.79 (d, J=8.5 Hz, 1H), 3.75 (s, 3H).

### Methyl isoindigo-6-carboxylate (6MCIId)

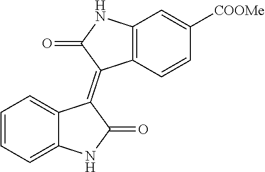 [SMILES: COC(=O)c1ccc2c(c1)NC(=O)/C2=C1/C(=O)Nc2ccccc21]

The target was synthesised by the general procedure starting with isatin (1.47 g, 10 mmol) and methyl 2-oxindole-6-carboxylate (1.91 g, 10 mmol) in a day to afford the desired product (2.93 g, 92%). ¹H NMR (400 MHz, DMSO) δ 11.08 (s, 1H), 10.94 (s, 1H), 9.15 (d, J=8.4 Hz, 1H), 9.07 (d, J=8.0 Hz, 1H), 7.57 (dd, J=8.4, 1.7 Hz, 1H), 7.38 (td, J=7.7, 1.2 Hz, 1H), 7.34 (d, J=1.6 Hz, 1H), 7.02-6.94 (m, 1H), 6.85 (d, J=7.3 Hz, 1H), 3.87 (s, 3H).

### Methyl 6-bromoisoindigo-6′-carboxylate (6B6MCIId)

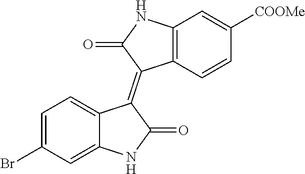 [SMILES: COC(=O)c1ccc2c(c1)NC(=O)/C2=C1/C(=O)Nc2cc(Br)ccc21]

The target was synthesised by the general procedure starting with 6-bromoisatin (1.13 g, 5 mmol) and methyl 2-oxindole-6-carboxylate (956 mg, 5 mmol) in a day to afford the desired product (1.74 g, 87%). ¹H NMR (400 MHz, DMSO) δ 11.12 (s, 1H), 11.09 (s, 1H), 9.14 (d, J=8.4 Hz, 1H), 9.02 (d, J=8.7 Hz, 1H), 7.57 (dd, J=8.4, 1.7 Hz, 1H), 7.33 (d, J=1.6 Hz, 1H), 7.20 (dd, J=8.7, 2.0 Hz, 1H), 7.01 (d, J=2.0 Hz, 1H), 3.87 (s, 3H).

### 5,5′,6,6′-Tetrafluoroisoindigo (5566TFIId)

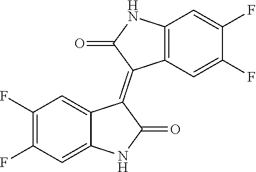 [SMILES: O=C1Nc2cc(F)c(F)cc2/C1=C1\C(=O)Nc2cc(F)c(F)cc21]

The target was synthesised by the general procedure starting with 5,6-difluoroindoline-2,3-dione (366.2 mg, 2 mmol) and 5,6-difluoroindolin-2-one (338.3 mg, 2 mmol) in three days to afford the desired product (345 mg, 52%). ¹H NMR (400 MHz, DMSO) δ 11.20 (s, 2H), 9.21 (dd, J=13.3, 8.7 Hz, 2H), 6.90 (dd, J=10.2, 7.1 Hz, 2H).

### Methyl-5-nitroisoindigo-6-carboxylate (5N6MCIId)

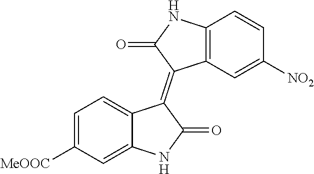 [SMILES: COC(=O)c1ccc2c(c1)NC(=O)/C2=C1/C(=O)Nc2ccc([N+](=O)[O-])cc21]

The target was synthesised by the general procedure starting with 5-nitroisatin (960.7 mg, 5 mmol) and methyl 2-oxindole-6-carboxylate (956 mg, 5 mmol) in three days to afford the desired product (1.77 g, 97%). ¹H NMR (400 MHz, DMSO) δ 11.68 (s, 1H), 11.22 (s, 1H), 10.10 (s, 1H), 9.15 (d, J=8.4 Hz, 1H), 8.30 (dd, J=8.7, 2.1 Hz, 1H), 7.57 (d, J=8.5 Hz, 1H), 7.33 (s, 1H), 7.03 (d, J=8.7 Hz, 1H), 3.87 (s, 3H).

### Dimethyl isoindigo-6,6′-dicarboxylate (66DMCIId)

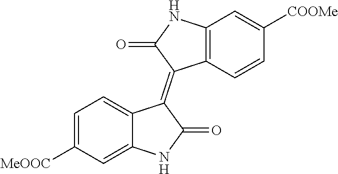 [SMILES: COC(=O)c1ccc2c(c1)NC(=O)/C2=C1/C(=O)Nc2cc(C(=O)OC)ccc21]

The target was synthesised by the general procedure starting with methyl isatin-6-carboxylate (615.5 mg, 3 mmol) and methyl 2-oxindole-6-carboxylate (573.6 mg, 3 mmol) in three days to afford the desired product (1.1 g, 97%). ¹H NMR (400 MHz, DMSO) δ 11.18 (s, 2H), 9.17 (d, J=8.2 Hz, 2H), 7.58 (d, J=7.9 Hz, 2H), 7.33 (s, 2H), 3.87 (s, 6H).

### 2-Oxoindoline-6-carboxylic Acid

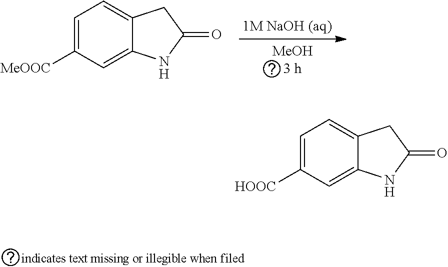 [SMILES: COC(=O)c1ccc2c(c1)NC(=O)C2 ; O=C1Cc2ccc(C(=O)O)cc2N1]

Methyl 2-oxindole-6-carboxylate (2 g, 10.4 mmol) was suspended in a mixture of methanol (20 mL) and 1 M NaOH (aq, 21 mL). The mixture was heated at reflux for 3 hours and allowed to cool down to room temperature. The solution was neutralised with 1 M HCl (aq) at 0° C. and a beige colour precipitate was formed. The precipitate was collected by filtration and washed with water and methanol. The crude product was dried in vacuo at 60° C. to give a beige solid (1.8 g, 97%), which was used in the next step without further purification. ¹H NMR (400 MHz, DMSO) δ 12.85 (s, 1H), 10.49 (s, 1H), 7.56 (d, J=7.6 Hz, 1H), 7.33-7.30 (2H), 3.55 (s, 2H).

### Isoindigo-6-carboxylic Acid (6CIId)

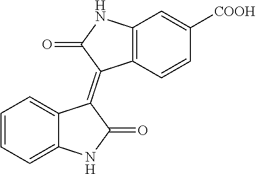 [SMILES: O=C1Nc2ccccc2/C1=C1\C(=O)Nc2cc(C(=O)O)ccc21]

The target was synthesised by the general procedure starting with isatin (1.47 g, 10 mmol) and 2-oxindoline-6-carboxylic acid (1.77 g, 10 mmol) in a day to afford the desired product (2.55 g, 83%). ¹H NMR (400 MHz, DMSO) δ 13.10 (s, 1H), 11.05 (s, 1H), 10.93 (s, 1H), 9.13 (d, J=8.4 Hz, 1H), 9.07 (d, J=8.1 Hz, 1H), 7.55 (dd, J=8.4, 1.7 Hz, 1H), 7.46-7.23 (m, 2H), 7.04-6.93 (m, 1H), 6.85 (d, J=7.2 Hz, 1H).

### 6-Bromoisoindigo-6′-carboxylic Acid (6BCIId)

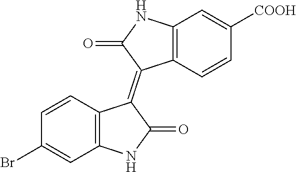 [SMILES: O=C1Nc2cc(Br)ccc2/C1=C1\C(=O)Nc2cc(C(=O)O)ccc21]

The target was synthesised by the general procedure starting with 6-bromoisatin (2.26 g, 10 mmol) and 2-oxindoline-6-carboxylic acid (1.77 g, 10 mmol) in a day to afford the desired product (3.52 g, 91%). ¹H NMR (400 MHz, DMSO) δ 13.15 (s, 1H), 11.11 (s, 1H), 11.10 (s, 1H), 9.11 (d, J=8.4 Hz, 1H), 9.01 (d, J=8.7 Hz, 1H), 7.54 (dd, J=8.4, 1.6 Hz, 1H), 7.31 (d, J=1.3 Hz, 1H), 7.18 (dd, J=8.7, 2.0 Hz, 1H), 6.98 (d, J=1.9 Hz, 1H).

### Sodium isoindigo-6-carboxylate (6CIIdNa)

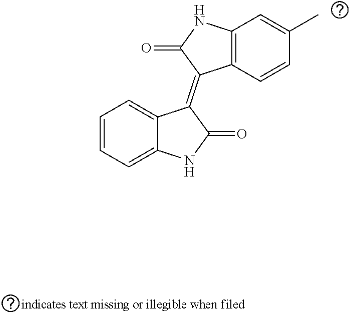 [SMILES: Cc1ccc2c(c1)NC(=O)/C2=C1/C(=O)Nc2ccccc21]

6CIId (306.3 mg, 1 mmol) was added portionwise to a solution of NaOH (0.2 M, 5 mL) in methanol at 0° C. The mixture was stirred for another 15 min at 0° C. before it was concentrated in vacuo to dryness to afford a brown solid (328 mg, >99%). ¹H NMR (400 MHz, DMSO) δ 10.84 (s, 2H), 9.05 (d, J=8.1 Hz, 1H), 8.93 (d, J=8.3 Hz, 1H), 7.40 (d, J=8.3 Hz, 1H), 7.35-7.24 (m, 2H), 6.95 (t, J=7.9 Hz, 1H), 6.83 (d, J=7.8 Hz, 1H).

### Methyl isoindigo-6-carbonylglycinate (6AIIdGly)

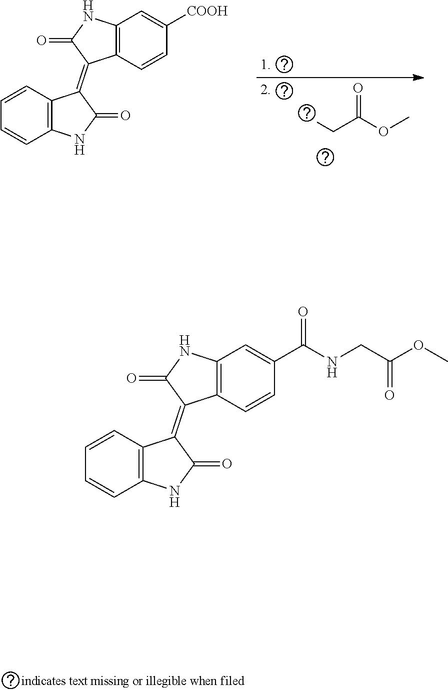

To a mixture of 6CIId (1.23 g, 4 mmol) and 2-(1H-benzotriazol-1-yl)-1,1,3,3-tetramethyluronium hexafluorophosphate (HBTU) (1.64 g, 4.32 mmol) in anhydrous DMF (25 mL) was added N,N-diisopropylethylamine (DIPEA) (12 mmol, 2.1 mL) dropwise. The mixture was stirred in Ar for 30 min before glycine methyl ester hydrochloride (540 mg, 4.32 mmol) in DMF (5 mL) was added. The reaction was monitored by TLC and stirred for another 15 h at room temperature. Upon completion of the reaction, the dark solution was poured onto a saturated NaHCO₃ aqueous solution (300 mL). The precipitate was collected by filtration, washed repeatedly with saturated NaHCO₃(aq), water, 0.2 M HCl (aq), cold methanol and dichloromethane, and dried in vacuo to give a dark red solid. The crude product was recrystallised from ethanol to afford a dark red powder (1.284 g, 85%). ¹H NMR (400 MHz, DMSO) δ 11.12 (s, 1H), 10.94 (s, 1H), 9.18-8.98 (m, 3H), 7.47 (dd, J=8.4, 1.6 Hz, 1H), 7.37 (td, J=7.7, 0.9 Hz, 1H), 7.29 (d, J=1.4 Hz, 1H), 7.03-6.94 (m, 1H), 6.85 (d, J=7.6 Hz, 1H), 4.02 (d, J=5.8 Hz, 2H), 3.67 (s, 3H). ¹³C NMR (101 MHz, DMSO) δ 170.26, 168.91, 168.81, 165.89, 144.50, 144.01, 136.57, 134.87, 133.22, 132.20, 129.65, 128.93, 124.24, 121.57, 121.27, 119.94, 109.67, 108.17, 51.75, 41.24.

### Methyl isoindigo-6-carbonylserinate (6AIIdSer)

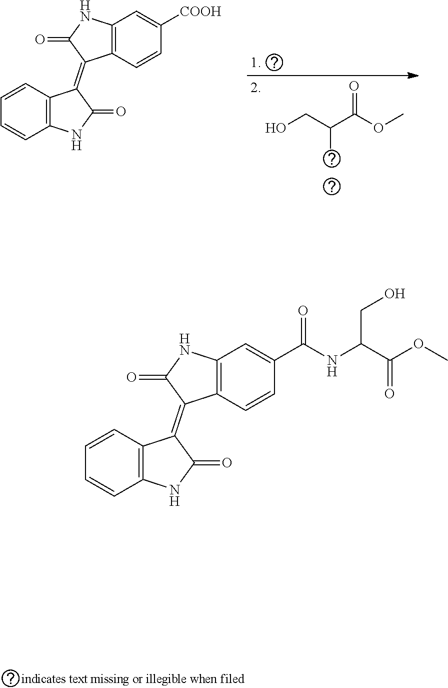

To a mixture of 6CIId (1.85 g, 6 mmol) and 2-(1H-benzotriazol-1-yl)-1,1,3,3-tetramethyluronium hexafluorophosphate (HBTU) (2.46 g, 6.48 mmol) in anhydrous DMF (30 mL) was added N,N-diisopropylethylamine (DIPEA) (3.15 mL, 18 mmol) dropwise. The mixture was stirred in Ar for 30 min before L-serine methyl ester hydrochloride (1.16 g, 6.48 mmol) in DMF (5 mL) was added. The reaction was monitored by TLC and stirred for another 15 h at room temperature. Upon completion of the reaction, the dark solution was poured onto a saturated NaHCO₃ aqueous solution (300 mL). The precipitate was collected by filtration, washed repeatedly with saturated NaHCO₃(aq), water, 0.2 M HCl (aq), cold methanol and dichloromethane, and dried in vacuo to give a dark red solid. The crude product was recrystallised from ethanol to afford a dark red powder (1.39 g, 57%). ¹H NMR (400 MHz, DMSO) δ 11.12 (s, 1H), 10.93 (s, 1H), 9.12 (d, J=8.4 Hz, 1H), 9.07 (d, J=7.9 Hz, 1H), 8.70 (d, J=7.3 Hz, 1H), 7.49 (dd, J=8.5, 1.4 Hz, 1H), 7.37 (t, J=7.2 Hz, 1H), 7.30 (s, 1H), 6.98 (t, J=7.6 Hz, 1H), 6.85 (d, J=7.6 Hz, 1H), 5.09 (t, J=6.1 Hz, 1H), 4.54 (dd, J=12.6, 5.5 Hz, 1H), 3.80 (t, J=5.7 Hz, 2H), 3.66 (s, 3H). ¹³C NMR (101 MHz, DMSO) δ 170.98, 168.98, 168.85, 165.90, 144.54, 143.96, 136.75, 134.90, 133.29, 132.27, 129.68, 128.89, 124.25, 121.61, 121.33, 120.28, 109.73, 108.38, 60.96, 55.74, 51.95.

### Methyl 6-bromoisoindigo-6′-carbonylserinate (6B6AIIdSer)

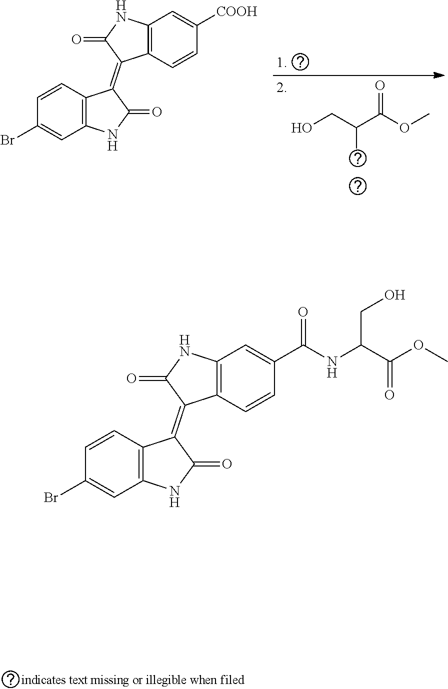

To a mixture of 6B6CIId (2.31 g, 6 mmol) and 2-(1H-benzotriazol-1-yl)-1,1,3,3-tetramethyluronium hexafluorophosphate (HBTU) (2.46 g, 6.48 mmol) in anhydrous DMF (30 mL) was added N,N-diisopropylethylamine (DIPEA) (3.15 mL, 18 mmol) dropwise. The mixture was stirred in Ar for 30 min before L-serine methyl ester hydrochloride (1.16 g, 6.48 mmol) in DMF (5 mL) was added. The reaction was monitored by TLC and stirred for another 15 h at room temperature. Upon completion of the reaction, the dark solution was poured onto a saturated NaHCO₃ aqueous solution (300 mL). The precipitate was collected by filtration, washed repeatedly with saturated NaHCO₃(aq), water, 0.2 M HCl (aq), cold methanol and dichloromethane, and dried in vacuo to give the pure solid product in dark red (2.14 g, 73%). ¹H NMR (400 MHz, DMSO) δ 11.16 (s, 1H), 11.09 (s, 1H), 9.11 (d, J=8.4 Hz, 1H), 9.02 (d, J=8.7 Hz, 1H), 8.71 (d, J=7.4 Hz, 1H), 7.49 (dd, J=8.4, 1.6 Hz, 1H), 7.30 (d, J=1.4 Hz, 1H), 7.20 (dd, J=8.6, 2.0 Hz, 1H), 7.01 (d, J=1.9 Hz, 1H), 5.07 (t, J=6.1 Hz, 1H), 4.53 (dd, J=12.7, 5.5 Hz, 1H), 3.81 (s, 2H), 3.66 (s, 3H).

### N,N′-Dipropyl-6,6′-dibromoisoindigo (NNDPr66DBIId)

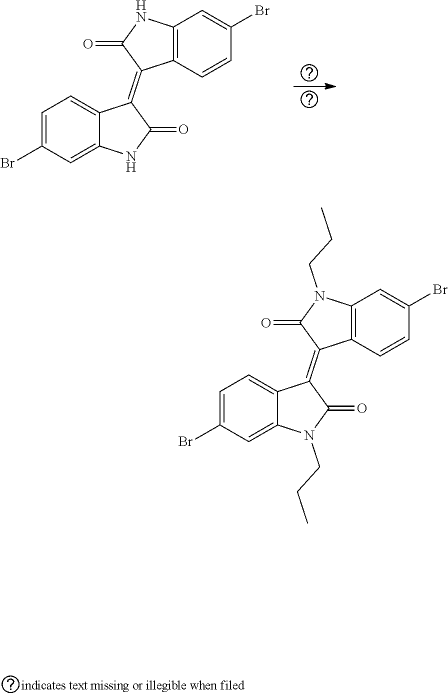 [SMILES: O=C1Nc2cc(Br)ccc2/C1=C1\C(=O)Nc2cc(Br)ccc21 ; CCCN1C(=O)/C(=C2/C(=O)N(CCC)c3cc(Br)ccc32)c2ccc(Br)cc21]

To a mixture of 66DBIId (158 mg, 0.377 mmol) and K₂CO₃ (261 mg, 1.89 mmol) in DMF (5 mL) under Ar was added propyl bromide (0.2 mL). The mixture was heated at 100° C. under Ar for 18 h. The reaction mixture was cooled and evaporated to dryness in vacuo. The crude solid was partition between dichloromethane and water. The organic phase was dried over Na₂SO₄, filtered, and dried in vacuo. The crude product was then purified by column chromatography on silica gel (230˜400 mesh, hexane/CH₂Cl₂=1:3) to give a red colour solid (104 mg, 55%). ¹H NMR (400 MHz, DMSO) δ 9.05 (d, J=8.6 Hz, 2H), 7.41 (d, J=1.9 Hz, 2H), 7.25 (dd, J=8.6, 1.9 Hz, 2H), 3.74 (t, J=7.0 Hz, 4H), 1.62 (dd, J=14.6, 7.6 Hz, 4H), 0.90 (t, J=7.4 Hz, 6H).

### N,N′-Di-(2-ethylhexyl)-isoindigo (NNDEHIId)

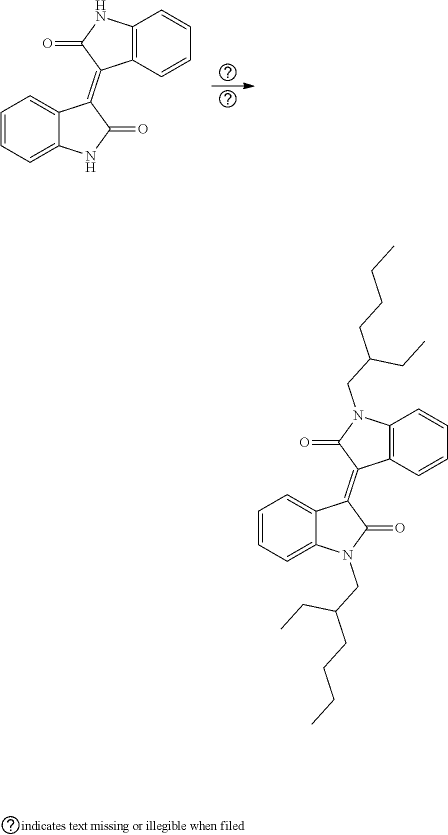 [SMILES: O=C1Nc2ccccc2/C1=C1\C(=O)Nc2ccccc21 ; CCCCC(CC)CN1C(=O)/C(=C2/C(=O)N(CC(CC)CCCC)c3ccccc32)c2ccccc21]

To a mixture of isoindigo (2.62 g, 10 mmol) and K₂CO₃ (6.9 g, 50 mmol) in DMF (100 mL) was added 1-bromo-2-ethylhexane (3.8 mL, 22 mmol) under Ar atmosphere. The mixture was heated at 100° C. for 1 day. After the reaction was cooled to room temperature, the mixture was poured onto 1 L of water and a dark red precipitate was formed. The solid was filtered, washed with a copious amount of water and cold methanol, and dried in vacuo. The crude product was purified by column chromatography on silica gel (230-400 mesh, hexane/CH₂Cl₂=1:1) to give a dark red solid (1.58 g, 32%). ¹H NMR (400 MHz, DMSO) δ 9.10 (d, J=7.6 Hz, 2H), 7.44 (t, J=7.7 Hz, 2H), 7.03 (dd, J=17.1, 8.0 Hz, 4H), 3.66 (d, J=7.6 Hz, 4H), 2.40 (d, J=10.6 Hz, 4H), 1.82 (s, 2H), 1.44-1.07 (m, 14H), 0.86 (dt, J=13.8, 7.2 Hz, 10H).

### 1.6.4 General Methods for Electrochemical Characterisations

Electrochemical measurements were conducted using a BioLogic VSP potentiostat (BioLogic Science Instruments). For the cyclic voltammetry measurements, glassy carbon (Ø=3 mm) was used as the working electrode, platinum wire was used as the counter electrode, silver wire was used as the pseudo-reference electrode and ferrocene was used as the internal reference. In a standard cyclic voltammetry measurement, the molecule of interest (2.5 mM) was dissolved in anhydrous DMF with 100 mM NBu₄PF₆ as the supporting salt. Typical cyclic voltammograms were collected at a scan rate of −50 mV s⁻¹. To study the effects of ionic species on the redox behaviour of the sorbent molecules, 100 mM LiClO₄, NaClO₄, KClO₄, NBu₄ClO₄, sodium triflate (NaOTf), or sodium bis(trifluoromethanesulfonyl)imide (NaTFSI) was used as the supporting electrolyte salt.

### 1.6.5 Bulk Electrolysis

IId²⁻ and IId-CO₂ adduct were synthesised by constant current bulk electrolysis of IId in DMSO-d₆ under N₂ or CO₂ atmosphere. The setup consisted of a three-neck round-bottom flask for the working, counter, and reference electrodes, respectively. Carbon felt (CT GF030; Fuel Cell Store) was used as the working electrode and a silver wire was used as the reference electrode. The counter electrode was separated from the isoindigo solution with a fritted electrode chamber (MR-1196; Bioanalytical Systems). A piece of carbon felt coated with LiFePO₄ (50 mg) was used as the counter electrode, which was immersed in a neat electrolyte (1 mL) without isoindigo. LiClO₄ (250 mM) in DMSO-d₆ was used as the electrolyte. In a standard condition, IId (26.2 mg, 0.1 mmol) was stirred and reduced at a constant current of 1.5 mA in DMSO-d₆ (2.5 mL, 250 mM LiClO₄) under continuous N₂ or CO₂ bubbling. The product was used for NMR analysis without further purification.

### 1.6.6 Nafion™ Membrane Pretreatment

The Nafion™ membranes (Nafion™ 115; Ionpower) were pretreated accordingly a previously reported method. Li et al., 2022. The membrane was boiled in 3% hydrogen peroxide for 1 h. The membranes were then boiled in 0.25 M sulfuric acid solution for 1 h and cleaned in boiling deionised water for 30 min (twice). Subsequently, the membranes were boiled in 0.25 M sodium hydroxide solution for 1 h and cleaned in boiling deionised water for 30 min (twice). Finally, the membranes were dried under vacuum at 80° C. for at least 1 d and stored in anhydrous DMF.

### 1.6.7 Flow-Based EMCC Prototype

In a typical experiment, the sorbent tank (20 mL scintillation vial with septum cap) was continuously bubbled with CO₂ feed gas (balanced by N₂) with the flow rate controlled by a mass flow controller (Alicat). For testing under simulated flue gas conditions, a gas mixture of 10% CO₂+3% O₂ balanced by N₂ was used. An infrared-based CO₂ sensor (SprintIR-W 100%) was connected at the gas exit to monitor the CO₂ concentration continuously. The tank headspace was filled with plastic beads (McMaster-Carr) to limit the mixing time in the headspace. The Fc tank was kept air free. A two-channel peristaltic pump (Masterflex) circulated the sorbent and the Fc electrolyte to a commercial flow cell (Scribner) at a flow rate of 10 mL min⁻¹. The flow cell utilised graphite plates with 5 cm² interdigitated flow fields pressed against two pieces of carbon paper electrodes (Sigracet 28 AA) at each side of the graphite electrodes to distribute the liquid flow. A Nafion™ 115 membrane was sandwiched by two pieces of polypropylene and placed between the carbon paper electrodes. The cell was kept sealed by Kalrez fluoropolymer elastomer gaskets (0.02 inches thick). CO₂ capture was conducted in a constant current mode while the release was carried out following a constant current/constant voltage protocol (that is, oxidizing the adduct at a constant current until a specific cut-off voltage followed by a constant voltage hold (the cut-off current for the constant voltage hold was 5% of the value used in the constant current oxidation). The cycling protocols of each experiment are provided in the corresponding figure captions.

For experiments using 6MCIId sorbent, we added 10 mM 6MCIId and 4 mM ferrocenium tetrafluoroboronate (FcBF₄) into the CE tank to mitigate sorbent crossover and facilitate the reduction of the oxidised ferrocenium in the counter electrolyte, respectively. For less soluble 55DBIId and 66DBIId, the sorbent was dispersed in DMAc to form a slurry catholyte.

### 1.6.8 DFT Calculations

The Gaussian16 code, Gaussian 16 Rev. C.01, 2016, was used in the DFT calculations with the B3LYP functional, Becke, 1993; Lee et al., 1988, and the 6-31++g(d,p) basis set. Clark et al., 1983; Hariharan and Pople, 1973; Hehre et al., 1972. The conductor-like polarizable continuum model (CPCM) was used to simulate the solvation effect of DMF solvent at room temperature. Barone and Cossi, 1998. To simulate the processes in solvated environment, free energies excluding translational contributions at 298 K are calculated. Benchmark of calculated redox potentials of the first and second electron transfer steps against measured potentials and comparison of functionals can be found in our previous work. Li et al., 2022. Vibrational frequencies were calculated for optimized structures by determining the second derivative of the energy with respect to the nuclear coordinates and then transforming to mass-weighted coordinates.

The equilibrium redox potential of the Lewis base sorbents (abbreviated as B) was calculated as (G(B_{red})−G(Bₒₓ))/(−|e|), where G(B_{red}) and G(Bₒₓ) are the Gibbs free energies of the oxidized and reduced base, respectively. The CO₂ binding energy (E_{binding}) was calculated as E_{binding}=G_{CO2@B}−G_{B}−G_{CO2}, where the first term on the right side of the equation is the Gibbs free energy of CO₂ adsorbed on the sorbent molecule, the second and third terms are the Gibbs energies of the isolated B (any charge state) and CO₂, respectively.

### 1.6.9 Cell Culture

NIH3T3/GFP fibroblast cell line was kindly provided by Dr. Yun Chen at Johns Hopkins University and cultured in Dulbecco's Modified Eagle's Medium (DMEM; Gibco) supplemented with 10% fetal bovine serum (FBS; Hyclone) and 1% penicillin/streptomycin (Pen/Strep; Gibco). U2OS.EGFP cells were kindly provided by Dr. J Keith Joung at Massachusetts General Hospital and cultured in DMEM supplemented with 10% FBS, 2 mM GlutaMAX (Gibco), and 1% Pen/Strep. MCF10A epithelial cell line was kindly provided by Dr. Konstantinos Konstantopoulos at Johns Hopkins University and cultured in DMEM/F-12 (1:1) supplemented with 5% New Zealand horse serum (Gibco), 20 ng mL⁻¹ EGF (PeproTech), 0.5 g mL⁻¹ hydrocortisone (Sigma Aldrich), 100 ng mL⁻¹ cholera toxin (Sigma Aldrich), 10 g mL⁻¹ insulin (Gibco), and 1% Pen/Strep. All cells were cultured in a humidified incubator at 5% CO₂ and 37° C.

### 1.6.10 Cytotoxicity Assay and Cell Morphology

Cell cytotoxicity was examined by alamarBlue HS Cell Viability Reagent (Invitrogen) according to manufacturer's protocol. Rampersad, 2012; Kumar et al., 2018. Briefly, cells were plated in 96 wellplates (Falcon) at a density of 20,000 per well for overnight. Testing chemicals dissolved in DMF were sterilised by passing through 0.2 m DMSO-safe Acrodisc syringe filters (Pall Laboratory) and then added into culture medium at predetermined concentrations. All testing conditions contained 1% DMF as the solvent background. The cells were incubated with the chemicals for 48 hours, washed once with Dulbecco's Phosphate Buffered Saline (DPBS; Gibco), and then incubated with fresh culture medium containing alamarBlue reagent for 4 hours in a humidified incubator at 37° C. and 5% CO₂. Absorbance was measured at 570 nm by a SpectraMax microplate reader, with 600 nm as a reference wavelength. The cell viability was calculated by relative absorbance to control groups. Data were presented as mean±standard deviation (SD). The dose-response curves were fitted using a four-parameter logistic function to calculate the cytotoxic median lethal concentration (LC₅₀). Cell morphology was monitored throughout the assay and images were acquired with an EVOS cell imaging system (Thermo Fisher).

TABLE 2
A summary of existing redox-active quinone sorbents with their redox potentials
(V vs. Fc^{+/}Fc) and CO₂ binding constants in DMF. Simeon et al., 2022.

| | | E_{1/2} (1)^{A} in | E_{1/2} (2)^{B} in | E_{1/2} (2) in | |
|---|---|---|---|---|---|
| Quinoids | | CO₂ (V) | N₂ (V) | CO₂ (V) | logK_{CO2} |
| AQ-O-C3H7 | 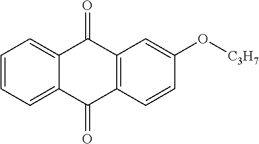 | −1.55 | −2.2 | −1.55 | 11.70 |
| AQ | 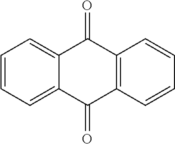 [SMILES: O=C1c2ccccc2C(=O)c2ccccc21] | −1.4 | −2.15 | −1.4 | 13.39 |
| AQ-Cl | 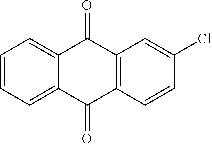 [SMILES: O=C1c2ccccc2C(=O)c2cc(Cl)ccc21] | −1.25 | −2 | −1.88 | 2.73 |
| AQ-COO- C3H7 | 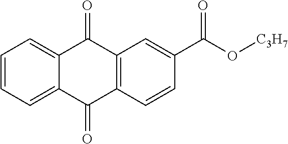 | −1.29 | −2 | −1.89 | 2.56 |
| BQ | 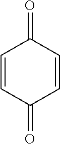 | −0.95 | −1.98 | −0.95 | 18.12 |
| DBQ | 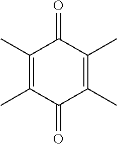 | −1.39 | −2.39 | −1.39 | 17.61 |
| TBQ | 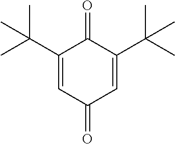 [SMILES: CC(C)(C)C1=CC(=O)C=C(C(C)(C)C)C1=O] | −1.25 | −2.29 | −1.25 | 18.29 |
| BQ-Cl2 | 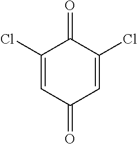 | −0.7 | −1.66 | −1.59 | 1.86 |
| BQ-Cl4 | 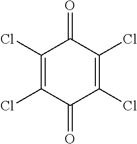 [SMILES: O=C1C(Cl)=C(Cl)C(=O)C(Cl)=C1Cl] | −0.48 | −1.42 | −1.38 | 1.28 |
| p-NQ | 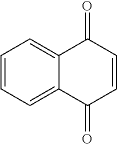 [SMILES: O=C1C=CC(=O)c2ccccc21] | −1.23 | −2.06 | −1.23 | 14.74 |
| p-NQ-Cl2 | 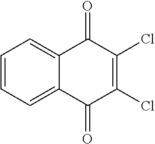 [SMILES: O=C1C(Cl)=C(Cl)C(=O)c2ccccc21] | −0.9 | −1.75 | −1.63 | 2.73 |

^{A}The first half-wave potential.
^{B}The second half-wave potential cathodic to the first one.

TABLE 3
Summary of key parameters of isoindigo sorbents including their redox properties, ¹H NMR, and optical band gap.

| | | | | | | | ¹H NMR | ¹H NMR | |
|---|---|---|---|---|---|---|---|---|---|
| | | E_{1/2} in | E_{1/2} in | | | | H^{a},) | (H^{b}, | Optical |
| | | N₂ | CO₂ | ΔEₚₑₐₖ(2)^{B} | K[CO₂] | | (H^{a,} | H^{b,}) | band |
| | Substituents | (V)^{A} | (V)^{A} | (V)^{A} | (M⁻¹) | logK[CO₂] | (ppm) | (ppm) | gap (eV) |
| 6CIIdNa | 6-COONa | −1.33, | −1.14 | 0.301 | 6.11E+05 | 5.79 | 9.05, | 10.84 | 2.03 |
| | | −1.49 | | | | | 8.93 | | |
| IId | — | −1.29, | −1.21 | 0.511 | 2.19E+09 | 9.34 | 9.06 | 10.89 | 2.04 |
| | | −1.79 | | | | | | | |
| 55DMIId | 5,5′-OMe | −1.29, | −1.2 | 0.494 | 1.13E+09 | 9.05 | 8.85 | 10.69 | 1.78 |
| | | −1.76 | | | | | | | |
| NNDEHIId | N,N′-2- | −1.23, | −1.16, | 0.350 | 4.19E+06 | 6.62 | 9.1 | N.A. | 2.00 |
| | ethylhexyl | −1.80 | −1.45 | | | | | | |
| 6BIId | 6-Br | −1.21, | −1.14 | 0.538 | 6.25E+09 | 9.8 | 9.04, | 10.95, | 2.05 |
| | | −1.75 | | | | | 9.00 | 11.06 | |
| 5BIId | 5-Br | −1.19, | −1.11 | 0.494 | 1.12E+09 | 9.05 | 9.07, | 10.96, | 2.04 |
| | | −1.67 | | | | | 9.31 | 11.05 | |
| 6AIIdSer | 6-methyl | −1.18, | −1.08 | 0.46 | 2.57E+08 | 8.41 | 9.07, | 10.94, | 2.04 |
| | serinate | −1.60 | | | | | 9.11 | 11.12 | |
| 6AIIdGly | 6-methyl | −1.17, | −1.08 | 0.469 | 4.30E+08 | 8.63 | 9.08, | 10.94, | 2.04 |
| | glycinate | −1.62 | | | | | 9.12 | 11.12 | |
| 55DFIId | 5,5′-F | −1.14, | −1.13 | 0.547 | 9.13E+09 | 9.96 | 9.00 | 11.02 | 1.97 |
| | | −1.69 | | | | | | | |
| 66DBIId | 6,6′-Br | −1.14, | −1.12 | 0.525 | 3.88E+04 | 9.59 | 8.99 | 11.1 | 2.06 |
| | | −1.67 | | | | | | | |
| 6MCIId | 6-COOMe | −1.12, | −1.09 | 0.484 | 7.78E+08 | 8.89 | 9.07, | 10.94, | 2.03 |
| | | −1.62 | | | | | 9.15 | 11.08 | |
| 6B6AIIdSer | 6-Br-6′-methyl | −1.11, | −1.11 | 0.475 | 5.41E+08 | 8.73 | 9.02, | 11.09, | 2.03 |
| | serinate | −1.55 | | | | | 9.11 | 11.16 | |
| 5566TFIId | 5,5′,6,6′-F | −1.10, | −1.1 | 0.570 | 2.17E+10 | 10.3 | 9.21 | 11.2 | 2.02 |
| | | −1.68 | | | | | | | |
| 55DBIId | 5,5-Br | −1.09, | −1.09 | 0.510 | 2.16E+09 | 9.33 | 9.32 | 11.11 | 1.99 |
| | | −1.61 | | | | | | | |
| 6CIId | 6-COOH | −1.09, | −1.06, | 0.129 | 7.50E+02 | 2.87 | 9.07, | 10.93, | 2.01 |
| | | −1.35 | −1.18 | | | | 9.13 | 11.05 | |
| 5NIId | 5-NO₂ | −1.08, | −1.08 | 0.484 | 7.63E+08 | 8.88 | 9.08, | 11.03, | 2.07 |
| | | −1.57 | | | | | 10.12 | 11.63 | |
| 5M5NIId | 5-OMe-5′- NO₂ | −1.08, | −1.08 | 0.470 | 4.45E+08 | 8.65 | 8.83, | 10.84, | 1.71 |
| | | −1.55 | | | | | 10.14 | 11.61 | |
| NNDPr66DBIId | N,N′-Pr-6,6′-Br | −1.07, | −1.08, | 0.247 | 7.48E+04 | 4.48 | 9.05 | N.A. | 2.02 |
| | | −1.61 | −1.34 | | | | | | |
| 6B6MCIId | 6-Br-6-COOMe | −1.05, | −1.05 | 0.486 | 8.34E+08 | 8.92 | 9.02, | 11.09, | 2.02 |
| | | −1.55 | | | | | 9.14 | 11.12 | |
| 66DMCIId | 6,6′-COOMe | −0.97, | −0.91 | 0.435 | 1.17E+08 | 8.07 | 9.17 | 11.18 | 2.02 |
| | | −1.41 | | | | | | | |
| 5N6MCIId | 5-NO₂-6-COOMe | −0.93, | −0.94, | 0.232 | 4.15E+04 | 4.62 | 9.15, | 11.22, | 2.08 |
| | | −1.40 | −1.15 | | | | 10.10 | 11.68 | |

^{A}Fc⁺/Fc is used as the internal reference.
^{B}ΔEₚₑₐₖ(2) was calculated based on the difference between the second reduction peak potentials (IId^{●-}/IId²⁻) in CO₂ and N₂.

TABLE 4
Reaction rate constant for the first CO₂ binding using
different isoindigo sorbents. Isoindigo (5 mM) in DMF using 0.1M
NBu₄PF₆ as the supporting salt.

| Isoindigo sorbent | k_{bimolecular} (M⁻¹ s⁻¹) | k_{f} (s⁻¹) (20% CO₂) |
|---|---|---|
| 6MCIId | 22.45 | 0.889 |
| 6AIIdGly | 19.85 | 0.786 |
| 66DBIID | 18.64 | 0.738 |
| NNDPr66DBIId | 3.31 | 0.131 |

TABLE 5
Comparison between the DFT-simulated 1^{st} electron-transfer
redox potentials (V vs. Fc⁺/Fc) and experimental 1^{st} half-wave potentials
in N₂ (V vs. Fc⁺/Fc) of various isoindigos.

| Isoindigo sorbent | Theoretical (V) | Experimental (V) |
|---|---|---|
| IId | −1.102 | −1.29 |
| 5BIId | −1.100 | −1.19 |
| 66DBIId | −1.058 | −1.14 |
| 55DBIId | −1.006 | −1.09 |

TABLE 6
Solubilities of various isoindigos in DMF.

| Isoindigo sorbent | Solubility in DMF (mM) |
|---|---|
| IId | 230 |
| 55DBIId | 6.3 |
| 66DMCIId | <2.5 |
| 6MCIId | 515 |
| 6AIIdGly | 606 |
| 6AIIdSer | 830 |
| 6B6AIIdSer | 568 |

### 1.7 Modular Synthesis of Isoindigos

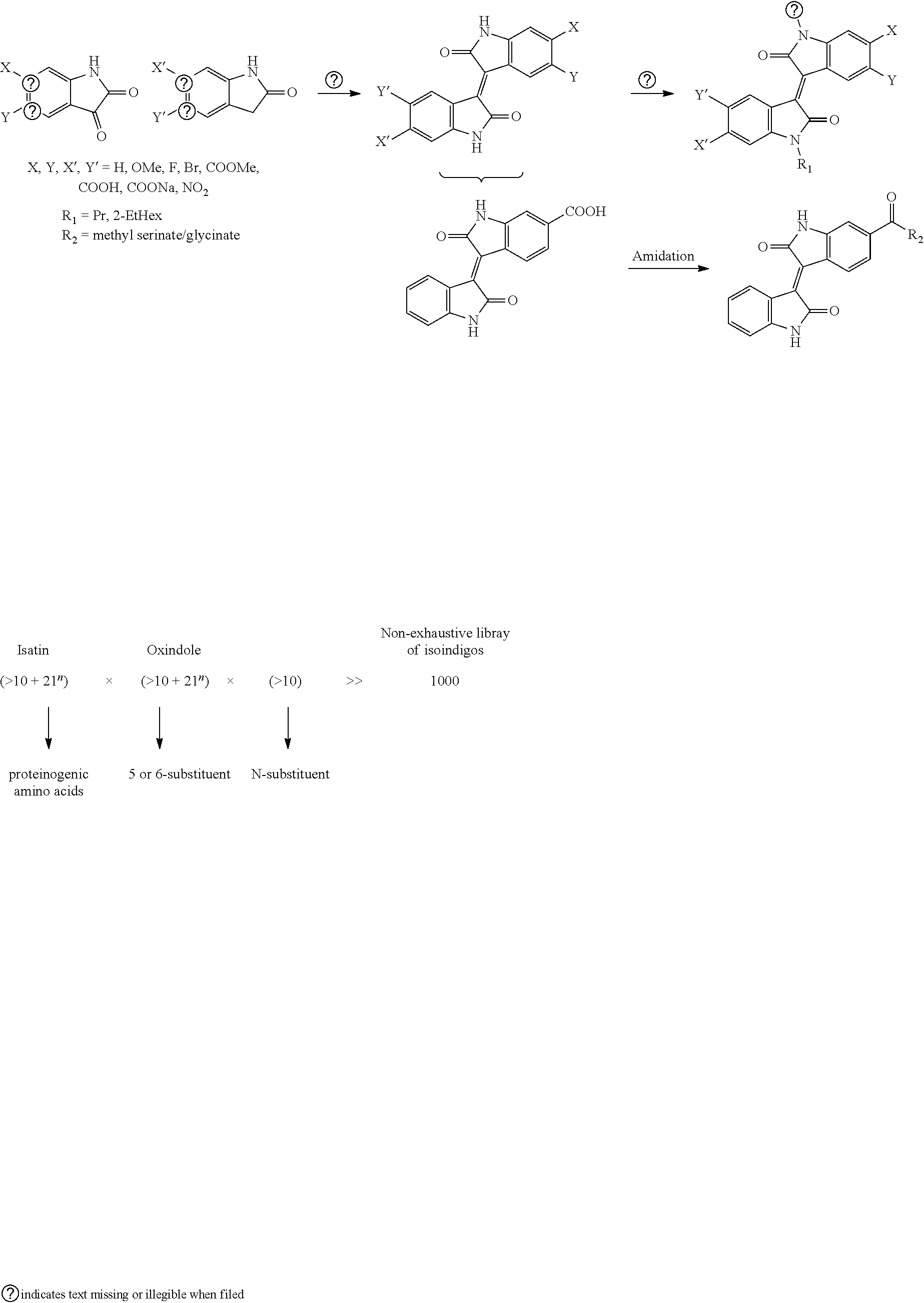

Through Knoevenagel condensation between 2-oxindoles and isatins, electron-donating or withdrawing groups (EDGs or EWGs) can be easily encoded into the aromatic systems of isoindigos at 5 or 6-position. The N-substituent groups can be introduced subsequently via S_{N}2 reactions. This ease in the synthesis and modification of isoindigo moieties allows us to quickly build a vast library of EMCC sorbent candidates with, in this Example, 21 representative compounds in total, where the redox potential, CO₂ binding constant, and solubility can be easily tuned via molecular engineering. Specifically, we explored a variety of substituents including electron-donating methoxy and electron-withdrawing fluoro, bromo, carboxyl, methyl carboxylate, amide, and nitro groups at 5 or 6-position of isoindigo to study the electronic and steric impact on their redox potentials and CO₂ affinities. N-substituents with aliphatic chains also have been probed to verify the role of intramolecular hydrogen bonding in CO₂ complexation. This design gives rise to a non-exhaustive library of isoindigos.

### 1.8 CO₂ Binding Constant and Reaction Rate Constant

The CO₂ binding constant (K_{CO2}) was calculated by Eq 1:

$${K}_{{CO}_{2}} = \frac{exp ( \frac{F}{RT} Δ {E}_{peak} ( 2 ) ) - 1}{[ {CO}_{2} ]} \qquad ( Eq 1 )$$

where F is the Faraday constant, R is the ideal gas constant, T is the temperature, ΔEₚₑₐₖ(2) is the difference between the second reduction peak potentials (IId^{·-}/IId²⁻) in CO₂ and N₂ atmospheres, and [CO₂] is the concentration of dissolved CO₂ (0.198 M in DMF under 100% CO₂ headspace at 298 K).

In electrochemistry, the Cottrell equation describes the current response (i) when the potential is a step function in time (t):

$$i = {{nAFC}_{0}^{*} ( \frac{D}{π t} )}^{\frac{1}{2}} \qquad ( Eq 2 )$$

where i is current, n is the number of electrons transferred per molecule, A is the area of the electrode, C*₀ is the bulk concentration of the redox-active isoindigo molecule, D is the diffusion coefficient, and t is time. The expected current for a diffusion limited electrochemical process following an ECE (electron transfer, chemical reaction, electron transfer) mechanism at a plane electrode under diffusion-controlled conditions is:

$$i = {{AFC}_{0}^{*} ( \frac{D}{π t} )}^{\frac{1}{2}} [ {n}_{1} + {n}_{2} ( 1 - {e}^{- {k}_{f} t} ) ] ; \qquad ( Eq 3 )$$

where n₁ and n₂ are the number of electrons transferred in the first and second step, respectively, and k_{f} is the forward reaction rate constant of the chemical reaction. Here, to calculate k_{f} after the first electron transfer, n₂ and n₁ are set to 1. Eq 3 then becomes:

$$i = {{AFC}_{0}^{*} ( \frac{D}{π t} )}^{\frac{1}{2}} ( 2 - {e}^{- {k}_{f} t} ) . \qquad ( Eq 4 )$$

The integrated Cottrell equation relates total charge Q with time for chronoamperometry:

$$Q = 2 {nAFD}^{\frac{1}{2}} {C}_{0}^{*} {π}^{- \frac{1}{2} t \frac{1}{2}} . \qquad ( Eq 5 )$$

Then, the constant k can be calculated, here k is assumed constant:

$$k = {nAFD}^{\frac{1}{2}} {C}_{0}^{*} {π}^{- \frac{1}{2}} ; \qquad ( Eq 6 )$$

where n is the number of electrons reduced per molecule.

To calculate k_{f}, the constant k is calculated using Eq 5 using potentiostatic measurements. Plugging in Eq 6, and rearranging Eq 4 gives:

$${k}_{f} t = - ln ( 2 - \frac{{it}^{\frac{1}{2}}}{k} ) . \qquad ( Eq 7 )$$

The data for determining k_{f} is shown in FIGS. 22 to 25 using 5 mM sorbent molecules in 0.1 M NBu₄PF₆ DMF electrolyte. The value k was determined using data under N₂; k_{f} was obtained under 1 bar 20% CO₂ (0.0396 M CO₂ in solution). The bimolecular reaction rate constant k_{bimolecular} was determined using Eq 8 and summarised in Table 3.

$${k}_{bimolecular} = {k}_{f} / [ {CO}_{2} ] ; \qquad ( Eq 8 )$$

where [CO₂]=0.0396 M.

### 1.9 CO₂ Binding Behaviours of 5N6MCIId and 6CIIdNa

We further designed an isoindigo with extremely strong EWG substituents to test the tolerance of the intramolecular hydrogen bonding (b) for promoting CO₂ binding. 5N6MCIId, with a nitro substitution at 5-position in one ring and a methyl carboxylate substitution at 6′-position in the other ring, exhibits the most anodically shifted reduction potential under N₂ (−0.93 V vs. Fc⁺/Fc) among all isoindigos in this work. Due to the non-symmetrical structure in 5N6MCIId, the strong electron-withdrawing carboxylate and nitro group can work synergistically to frustrate the CO₂ binding in one oxindole ring. The carboxylate group withdraws the electron density from the O atom within the ring connecting it, while the nitro group in the adjacent ring creates steric hindrance and dipole-dipole repulsion to inhibit CO₂ binding (FIG. 27). In addition, the methyl carboxylate group is also a hydrogen bonding acceptor which may disrupt the intermolecular hydrogen bonding (b) between the amide hydrogen and the CO₂ complexed. As a result, the molecule displays a weaker binding with CO₂ compared to the other N-unsubstituted isoindigos with a log KCO2cof 4.62. Nevertheless, this value is qualified for CO₂ capture from flue gas, and the reductive potential required for completing two-electron transfer is −1.15 V vs. Fc⁺/Fc, meaning it still can circumvent the oxygen-related side reactions during the EMCC process.

In addition to EDG and EWG substituents, we also study the effect of ionic substituents on the isoindigo moiety. We synthesised an isoindigo (6CIIdNa) bearing sodium carboxylate group at 6-position (FIG. 3). Despite the electron-withdrawing effect of the substituent, 6CIIdNa displays a reduction potential at −1.33 V vs. Fc⁺/Fc under N₂ and a CO₂ binding constant of 5.79. The cathodically shifted potential can be explained by the electrostatic repulsion between the negatively charged electrode surface and the anionic 6CIIdNa which creates an energy barrier to reduce the molecule to species with more negative charges. The decreased CO₂ binding affinity is probably caused by the disruption of the intermolecular hydrogen bonding (b) between the amide hydrogen and the CO₂ complexed, since the carboxylate anion is a strong hydrogen bonding acceptor.

### 1.10 Degradation Rate of EMCC Prototype Based on 6B6AIIdSer

The decay of the flow-based EMCC prototype may come from many different factors, such as crossover of the redox-active electrode molecules, decomposition of the electrolyte or carbon paper, or degradation of the CO₂ carrier. For 6B6AIIdSer (FIG. 41), we utilise the energy capacity of the sorbent tank as a reference to estimate the degradation rate using the following equation,

$${r}_{d} = ( \frac{{Q}_{0} - {Q}_{n}}{n \cdot {Q}_{0}} ) * 100 \% ;$$

where rd is the degradation rate, Q₀ is the initial energy capacity of the sorbent tank, Qₙ is the last-cycle energy capacity of the sorbent tank, and n is the cycle number.

Using −1.3 V as the cut-off potential, the energy capacity of the flow cell decreased to 7 mAh in the last cycle tested. The initial energy capacity of 6B6AIIdSer (0.2 M, 5 mL) is 53.6 mAh. Therefore, the estimated degradation rate is approximately 4.62%.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Mac Dowell, N., Fennell, P. S., Shah, N. and Maitland, G. C. The role of CO₂ capture and utilization in mitigating climate change. Nat. Clim. Change 7, 243-249 (2017).
Deutz, S. and Bardow, A. Life-cycle assessment of an industrial direct air capture process based on temperature-vacuum swing adsorption. Nat. Energy 6, 203-213 (2021).
Bui, M. et al. Carbon capture and storage (CCS): the way forward. Energy Environ. Sci. 11, 1062-1176 (2018).
Chu, S. Carbon capture and sequestration. Science 325, 1599-1599 (2009).
Sanz-Pérez, E. S., Murdock, C. R., Didas, S. A. and Jones, C. W. Direct capture of CO₂ from ambient air. Chem. Rev. 116, 11840-11876 (2016).
   Sharifian, R., Wagterveld, R. M., Digdaya, I. A., Xiang, C. and Vermaas, D. A. Electrochemical carbon dioxide capture to close the carbon cycle. Energy Environ. Sci. 14, 781-814 (2021).
Liu, Y., Lucas, E., Sullivan, I., Li, X. and Xiang, C. Challenges and opportunities in continuous flow processes for electrochemically mediated carbon capture. iScience 25, 105153 (2022).
Rheinhardt, J. H., Singh, P., Tarakeshwar, P. and Buttry, D. A. Electrochemical capture and release of carbon dioxide. ACS Energy Lett. 2, 454-461 (2017).
Renfrew, S. E., Starr, D. E. and Strasser, P. Electrochemical approaches toward CO₂ capture and concentration. ACS Catalysis 10, 13058-13074 (2020).
Barlow, J. M. et al. Molecular design of redox carriers for electrochemical CO₂ capture and concentration. Chem. Soc. Rev. 51, 8415-8433 (2022).
Diederichsen, K. M. et al. Electrochemical methods for carbon dioxide separations. Nat. Rev. Methods Primers 2, 68 (2022).
Bell, W. L., Miedaner, A., Smart, J. C., DuBois, D. L. and Verostko, C. E. Synthesis and evaluation of electroactive CO₂ carriers. SAE Transactions 97, 544-552 (1988).
Mizen, M. B. and Wrighton, M. S. Reductive addition of CO₂ to 9,10-phenanthrenequinone. J. Electrochem. Soc. 136, 941-946 (1989).
Nagaoka, T., Nishii, N., Fujii, K. and Ogura, K. Mechanisms of reductive addition of CO₂ to quinones in acetonitrile. J. Electroanal. Chem. 322, 383-389 (1992).
Rahimi, M., Khurram, A., Hatton, T. A. and Gallant, B. Electrochemical carbon capture processes for mitigation of CO₂ emissions. Chem. Soc. Rev. 51, 8676-8695 (2022).
Scovazzo, P., Poshusta, J., DuBois, D., Koval, C. and Noble, R. Electrochemical separation and concentration of <1% carbon dioxide from nitrogen. J. Electrochem. Soc. 150, D91 (2003).
Gurkan, B., Simeon, F. and Hatton, T. A. Quinone reduction in ionic liquids for electrochemical CO₂ separation. ACS Sustain. Chem. Eng. 3, 1394-1405 (2015).
Voskian, S. and Hatton, T. A. Faradaic electro-swing reactive adsorption for CO₂ capture. Energy Environ. Sci. 12, 3530-3547 (2019).
Diederichsen, K. M., Liu, Y., Ozbek, N., Seo, H. and Hatton, T. A. Toward solvent-free continuous-flow electrochemically mediated carbon capture with high-concentration liquid quinone chemistry. Joule 6, 221-239 (2021).
Li, X., Zhao, X., Liu, Y., Hatton, T. A. and Liu, Y. Redox-tunable Lewis bases for electrochemical carbon dioxide capture. Nat. Energy 7, 1065-1075 (2022).
Simeon, F. et al. Electrochemical and molecular assessment of quinones as CO₂-binding redox molecules for carbon capture. J. Phys. Chem. C 126, 1389-1399 (2022).
Barlow, J. M. and Yang, J. Y. Oxygen-stable electrochemical CO₂ capture and concentration with quinones using alcohol additives. J. Am. Chem. Soc. 144, 14161-14169 (2022).
Bui, A. T., Hartley, N. A., Thom, A. J. W. and Forse, A. C. Trade-off between redox potential and the strength of electrochemical CO₂ capture in quinones. J. Phys. Chem. C 126, 14163-14172 (2022).
Hansch, C., Leo, A. and Taft, R. W. A survey of Hammett substituent constants and resonance and field parameters. Chem. Rev. 91, 165-195 (1991).
Wang, E., Mammo, W. and Andersson, M. R. 25th anniversary article: isoindigo-based polymers and small molecules for bulk heterojunction solar cells and field effect transistors. Adv. Mater. 26, 1801-1826 (2014).
Bogdanov, A. V. and Mironov, V. F. Recent advances in the application of isoindigo derivatives in materials chemistry. Beilstein J. Org. Chem. 17, 1533-1564 (2021).
Kiss, F. L., Corbet, B. P., Simeth, N. A., Feringa, B. L. and Crespi, S. Predicting the substituent effects in the optical and electrochemical properties of N,N′-substituted isoindigos. Photochem. Photobiol. Sci. 20, 927-938 (2021).
Wei, X., Zhang, W. and Yu, G. Semiconducting polymers based on isoindigo and its derivatives: synthetic tactics, structural modifications, and applications. Adv. Funct. Mater. 31, 2010979 (2021).
Xu, Y. et al. Assessing the kinetics of quinone-CO₂ adduct formation for electrochemically mediated carbon capture. ACS Sustain. Chem. Eng. 11, 11333-11341 (2023).
Schimanofsky, C. et al. Direct electrochemical CO₂ capture using substituted anthraquinones in homogeneous solutions: a joint experimental and theoretical study. J. Phys. Chem. C 126, 14138-14154 (2022).
del Rio, B. et al. The biogenic amines putrescine and cadaverine show in vitro cytotoxicity at concentrations that can be found in foods. Sci. Rep. 9, 120 (2019).
Rohr, A. C. et al. Potential toxicological effects of amines used for carbon capture and storage and their degradation products. Energy Procedia 37, 759-768 (2013).
Eisaman, M. D. et al. CO₂ separation using bipolar membrane electrodialysis. Energy Environ. Sci. 4, 1319-1328 (2011).
Wang, M. et al. Flue gas CO₂ capture via electrochemically mediated amine regeneration: System design and performance. Appl. Energy 255, 113879 (2019).
Wang, M., Herzog, H. J. and Hatton, T. A. CO₂ capture using electrochemically mediated amine regeneration. Ind. Eng. Chem. Res. 59, 7087-7096 (2020).
Jin, S., Wu, M., Gordon, R. G., Aziz, M. J. and Kwabi, D. G. pH swing cycle for CO₂ capture electrochemically driven through proton-coupled electron transfer. Energy Environ. Sci. 13, 3706-3722 (2020).
Jin, S., Wu, M., Jing, Y., Gordon, R. G. and Aziz, M. J. Low energy carbon capture via electrochemically induced pH swing with electrochemical rebalancing. Nat. Commun. 13, 2140 (2022).
   Liu, Y., Ye, H.-Z., Diederichsen, K. M., Van Voorhis, T. and Hatton, T. A. Electrochemically mediated carbon dioxide separation with quinone chemistry in salt-concentrated aqueous media. Nat. Commun. 11, 2278 (2020).
Gaussian 16 Rev. C.01 (Wallingford, CT, 2016).
Becke, A. D. Density-functional thermochemistry. III. The role of exact exchange. J. Chem. Phys. 98, 5648-5652 (1993).
Lee, C., Yang, W. & Parr, R. G. Development of the Colle-Salvetti correlation-energy formula into a functional of the electron density. Phys. Rev. B 37, 785-789 (1988).
Clark, T., Chandrasekhar, J., Spitznagel, G. W. & Schleyer, P. V. R. Efficient diffuse function-augmented basis sets for anion calculations. III. The 3-21+G basis set for first-row elements, Li—F. J. Comput. Chem. 4, 294-301 (1983).
Hariharan, P. C. & Pople, J. A. The influence of polarization functions on molecular orbital hydrogenation energies. Theor. Chim. Acta 28, 213-222 (1973).
Hehre, W. J., Ditchfield, R. & Pople, J. A. Self-consistent molecular orbital methods. XII. Further extensions of gaussian-type basis sets for use in molecular orbital studies of organic molecules. J. Chem. Phys. 56, 2257-2261 (1972).
Barone, V. & Cossi, M. Quantum calculation of molecular energies and energy gradients in solution by a conductor solvent model. J. Phys. Chem. A 102, 1995-2001 (1998).
Rampersad, S. N. Multiple Applications of Alamar Blue as an Indicator of Metabolic Function and Cellular Health in Cell Viability Bioassays. Sensors 12, 12347-12360 (2012).
Kumar, P., Nagarajan, A. & Uchil, P. D. Analysis of cell viability by the alamarBlue assay. Cold Spring Harb. Protoc. 6, 462-464 (2018).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 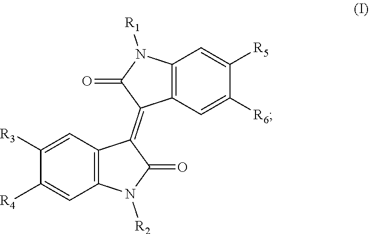
wherein:
R₁ and R₂ are each independently selected from H and branched or straightchain C₁-C₈ alkyl;
R₃, R₄, and R₆ are each independently selected from H, an electron withdrawing group, and an electron donating group;
R₅ is selected from H, an electron withdrawing group, an electron donating group, —COR_{y}, wherein R_{y} is an amino acid or a substituted amino acid, and —C(═O)—OR_{z}, wherein R_{z} is selected from H and branched or straightchain C₁-C₄ alkyl;
provided that at least one of R₁, R₂, R₃, R₄, R₅, and R₆ is not H; and
acceptable salts thereof.

2. The compound of claim 1, wherein the electron donating group is selected from alkoxide, hydroxyl, alkoxyl, amine, amide, ester, and alkyl.

3. The compound of claim 1, wherein the electron withdrawing group is selected from halogen, cyano, nitro, haloalkyl, ammonium, carbonyl, and sulfonyl.

4. The compound of claim 1, wherein:
R₁ and R₂ are each independently selected from H and branched or straightchain C₁-C₈ alkyl;
R₃, R₄, and R₆ are each independently selected from H, branched or straightchain C₁-C₄ alkyl, halogen, C₁-C₄ alkoxyl, nitro, and —C(═O)—OR₇, wherein R₇ is branched or straightchain C₁-C₄ alkyl;
R₅ is selected from H, branched or straightchain C₁-C₄ alkyl, halogen, C₁-C₄ alkoxyl, nitro, —C—OR₈, wherein R₈ is selected from an amino acid, a substituted amino acid, and —NHR₉, wherein R₉ is —CHR₁₀—C(═O)—O—Rn, wherein R₁₀ is H or —CH₂—OH, and Ru is branched or straightchain C₁-C₄ alkyl, and —C(═O)—OR₁₂, wherein R₁₂ is selected from H and branched or straightchain C₁-C₄ alkyl;
provided that (i) at least one of R₁, R₂, R₃, R₄, R₅, and R₆ is not H, and (ii) R₄ and R₅ cannot both be Br at the same time if R₁, R₂, R₃, and R₆ are each H; and
acceptable salts thereof.

5. The compound of claim 4, wherein R₁ and R₂ are each H.

6. The compound of claim 4, wherein R₁ and R₂ are each branched or straightchain C₁-C₈ alkyl.

7. The compound of claim 6, wherein R₁ and R₂ are each —CH₂CH₂—CH₃ or
—CH₂CH(CH₂CH₃)(CH₂CH₂CH₂CH₃).

8. The compound of claim 4, wherein one or more of R₃, R₄, R₅, and R₆ is selected from branched or straightchain C₁-C₄ alkyl, C₁-C₄ alkoxyl, halogen, nitro, —C—OR₈, wherein R₈ is selected from an amino acid, a substituted amino acid, and —NHR₉, wherein R₉ is —CHR₁₀—C(═O)—O—R₁₁, wherein R₁₀ is H or —CH₂—OH, and Ru is branched or straightchain C₁-C₄ alkyl, and —C(═O)—OR₁₂, wherein R₁₂ is selected from H and branched or straightchain C₁-C₄ alkyl;

9. The compound of claim 4, wherein:
(i) R₃ and R₆ are each the same and are not H;
(ii) R₄ and R₅ are the each same and are not H; or
(iii) R₃, R₄, R₅, and R₆ are each the same and are not H.

10. The compound of claim 4, wherein:
(i) R₃ is halogen and R₄, R₅, and R₆ are each H;
(ii) R₄ is halogen and R₃, R₅, and R₆ are each H;
(iii) R₅ is —C(═O)—OR₁₂, wherein R₁₂ is H or branched or straightchain C₁-C₄ alkyl, R₃ and R₆ are each H, and R₄ is H or halogen; or
(iv) R₆ is nitro and R₅ is H, R₃ is H or C₁-C₄ alkoxyl, and R₄ is H or —C(═O)—OR₁₂, wherein R₈ is H or branched or straightchain C₁-C₄ alkyl.

11. The compound of claim 4, wherein the compound of formula (I) is selected from:

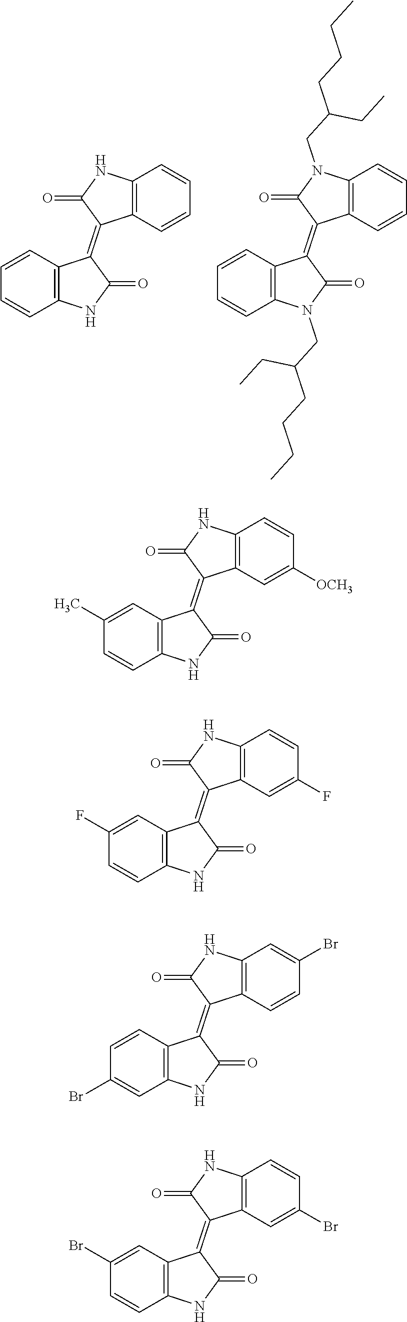

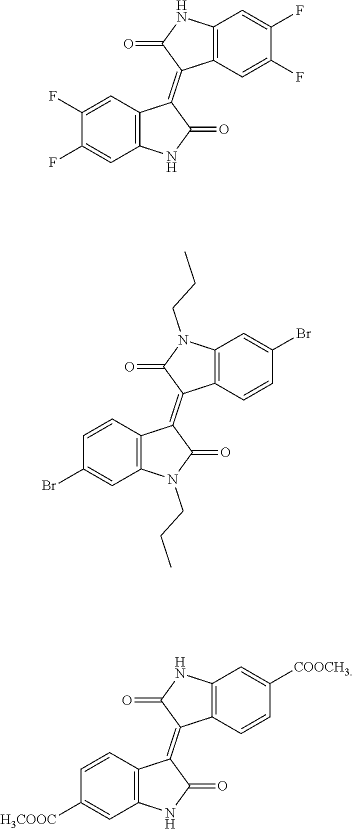

12. The compound of claim 4, wherein the compound of formula (I) is selected from:

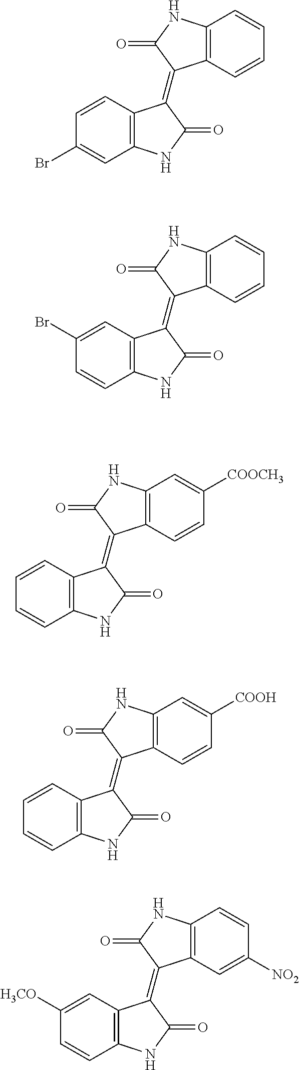 [SMILES: O=C1Nc2ccccc2/C1=C1\C(=O)Nc2cc(Br)ccc21 ; O=C1Nc2ccccc2/C1=C1\C(=O)Nc2ccc(Br)cc21 ; COC(=O)c1ccc2c(c1)NC(=O)/C2=C1/C(=O)Nc2ccccc21 ; O=C1Nc2ccccc2/C1=C1\C(=O)Nc2cc(C(=O)O)ccc21 ; COc1ccc2c(c1)/C(=C1\C(=O)Nc3ccc([N+](=O)[O-])cc31)C(=O)N2]

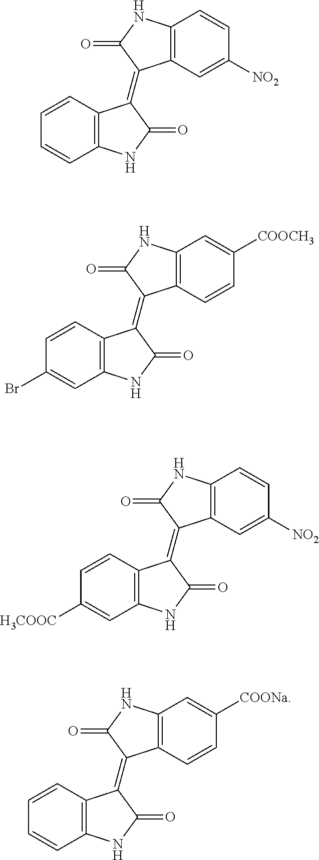

13. The compound of claim 4, wherein the compound of formula (I) comprises a compound of formula (Ia): 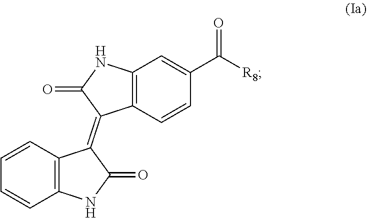
wherein:
R₈ is —NHR₉, wherein R₉ is —CHR₁₀—C(═O)—O—R₁₁, wherein R₁₀ is H or —CH₂—OH, and R₁₁ is branched or straightchain C₁-C₄ alkyl.

14. The compound of claim 13, wherein the compound is selected from:

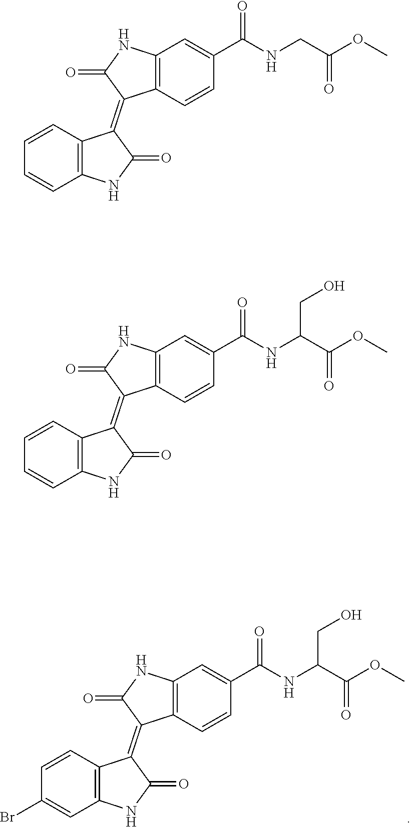 [SMILES: COC(=O)CNC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2ccccc21 ; COC(=O)C(CO)NC(=O)c1ccc2c(c1)NC(=O)/C2=C1/C(=O)Nc2cc(Br)ccc21]

15. A sorbent material comprising a compound of claim 1.

16. A process for capturing CO₂ from a gas sample, the process comprising contacting the gas sample with a compound of claim 1.

17. The process of claim 16, wherein the process comprises an electrochemically mediated carbon capture (EMCC) process.

18. The process of claim 17, comprising:
(i) electro-reduction of the sorbent to form an adduct with CO₂; and
(ii) oxidising the adduct to liberate CO₂ regenerate the sorbent.

19. The process of claim 16, wherein the gas sample is selected from ambient air, ventilated air, and a stream of gas.

20. The process of claim 16, wherein the process is a fixed-bed process or a flow-based process.

21. A system for separating carbon dioxide from ambient air or a stream of gases, the system comprising the compound of claim 1.
